(19) United States
(12) Patent Application Publication
(10) Pub. No.: US 2025/0043265 A1
(43) Pub. Date: Feb. 6, 2025

(54) HYBRID CLUCOAMYLASES AND METHODS OF USE THEREOF

(71) Applicant: DANISCO US INC., Palo Alto, CA (US)

(72) Inventors: Zhongmei TANG, Shanghai (CN); Jing GE, Shanghai (CN); Zhenghong ZHANG, Shanghai (CN); Yuepeng SHANG, Shanghai (CN)

(21) Appl. No.: 18/718,371

(22) PCT Filed: Dec. 21, 2022
(86) PCT No.: PCT/US2022/082158

§ 371 Date: Jun. 10, 2024

(30) Foreign Application Priority Data
Dec. 24, 2021 (WO) PCT/CN2021/141254

(51) Int. Cl. C12N 9/34; C07K 14/705; C12C 5/00; C12N 1/16; C12P 21/02

(52) U.S. Cl. CPC C12N 9/2428; C07K 14/705; C12C 5/004; C12N 1/165; C12P 21/02; C12Y 302/01003

(57) ABSTRACT

Described herein, inter alia, are hybrid glucoamylase polypeptides derived from glucoamylases from Zygomycetes (e.g. Mucorales). Additionally, the disclosure also relates to processes for using the hybrid glucoamylase polypeptides disclosed herein for producing fermentation products as well as methods for increasing starch digestibility in an animal and in methods for producing fermented beverages.

### CROSS-REFERENCE TO RELATED APPLICATIONS

This application claims priority to International Patent Application No. PCT/CN2021/141254, filed Dec. 24, 2021, the disclosure of which is incorporated by reference herein in its entirety.

### REFERENCE TO AN ELECTRONIC SEQUENCE LISTING

The contents of the electronic sequence listing (NB41866WOPCT2_sequencelisting.xml; Size: 288 kilobytes; and Date of Creation: Dec. 19, 2022) is herein incorporated by reference in its entirety.

### FIELD OF THE INVENTION

The present disclosure relates compositions comprising hybrid glucoamylase polypeptides and methods for saccharifying a starch substrate as well as methods for producing fermentation products using the same.

### BACKGROUND

Glucoamylase (1,4-alpha-D-glucan glucohydrolase, EC 3.2.1.3) is an enzyme which catalyzes the release of D-glucose from the non-reducing ends of starch or related oligo- and poly-saccharide molecules. Glucoamylases are produced by several filamentous fungi and yeast.

The major application of glucoamylase is the saccharification of partially processed starch/dextrin to glucose, which is an essential substrate for numerous fermentation processes. The glucose may then be converted directly or indirectly into a fermentation product using a fermenting organism. Examples of commercial fermentation products include alcohols (e.g., ethanol, methanol, butanol, 1,3-propanediol); organic acids (e.g., citric acid, acetic acid, itaconic acid, lactic acid, gluconic acid, gluconate, lactic acid, succinic acid, 2,5-diketo-D-gluconic acid); ketones (e.g., acetone); amino acids (e.g., glutamic acid); gases (e.g., H2 and CO2), and more complex compounds.

The end product may also be syrup. For instance, the end product may be glucose, but may also be converted, e.g., by glucose isomerase to fructose or a mixture composed almost equally of glucose and fructose. This mixture, or a mixture further enriched with fructose, is the most commonly used high fructose corn syrup (HFCS) commercialized throughout the world.

Glucoamylase for commercial purposes has traditionally been produced employing filamentous fungi, although a diverse group of microorganisms is reported to produce glucoamylase since they secrete large quantities of the enzyme extracellularly. However, commercially used fungal glucoamylases have certain limitations such as slow catalytic activity or lack of stability that increase process costs.

There continues to be a need for new glucoamylases and glucoamylase variants to improve the efficiency of saccharification and provide a high yield in fermentation products.

### SUMMARY

The present disclosure relates to the identification and construction of hybrid (a.k.a. “chimeric”) glucoamylase polypeptides derived from glucoamylases from Zygomycetes (e.g. Mucorales). Additionally, the disclosure also relates to processes for using the hybrid glucoamylase polypeptides disclosed herein for producing fermentation products as well as methods for increasing starch digestibility in an animal and in methods for producing fermented beverages.

In some aspects, provided herein is a hybrid polypeptide comprising: a) a first amino acid sequence comprising a catalytic module having glucoamylase activity or a functional fragment thereof; and b) a second amino acid sequence comprising a carbohydrate-binding module (CBM) or a functional fragment thereof, wherein i) the first amino acid sequence comprising a catalytic module having glucoamylase activity or a functional fragment thereof is derived from Zygomycetes; and ii) the second amino acid sequence is located at the N- and/or C-terminus of the first amino acid sequence. In some embodiments, the first amino acid sequence has at least about 60%, at least about 65%, at least about 70%, at least about 75%, at least about 80%, at least about 85%, at least about 90%, at least about 95%, at least about 97%, at least about 98%, at least about 99%, or about 100% sequence identity to the amino acid sequence of SEQ ID NO: 3, SEQ ID NO: 33, SEQ ID NO: 41, SEQ ID NO:47, SEQ ID NO:49, SEQ ID NO: 53, SEQ ID NO: 55, SEQ ID NO:59, SEQ ID NO: 61, SEQ ID NO: 65, SEQ ID NO: 67, SEQ ID NO: 71, SEQ ID NO: 73, SEQ ID NO: 105, SEQ ID NO: 110, SEQ ID NO: 115, SEQ ID NO: 120, or SEQ ID NO: 125. In some embodiments of any of the embodiments disclosed herein, the carbohydrate-binding module belongs to a carbohydrate binding module family selected from the group consisting of CBM20, CBM21, CBM25, CBM26, CBM34, CBM41 and CBM45. In some embodiments, the polypeptide comprises a CBM at the N- and C-terminus of the first amino acid sequence. In some embodiments of any of the embodiments disclosed herein, the second amino acid sequence has at least about 70%, at least about 75%, at least about 80%, at least about 85%, at least about 90%, at least about 95%, at least about 97%, at least about 98%, at least about 99%, or about 100% sequence identity to one or more amino acid sequence selected from the group consisting of amino acids 25-130 of SEQ ID NO: 1, 24-130 of SEQ ID NO: 5, 22-130 of SEQ ID NO: 7, 24-130 of SEQ ID NO: 9, 24-130 of SEQ ID NO: 11, 22-130 of SEQ ID NO: 13, 25-130 of SEQ ID NO: 15, 26-131 of SEQ ID NO: 17, 25-130 of SEQ ID NO: 19, 24-127 of SEQ ID NO: 21, 23-145 of SEQ ID NO: 23, 28-131 of SEQ ID NO: 25, SEQ ID NO: 31, and SEQ ID NO: 39. In some embodiments of any of the embodiments disclosed herein, the polypeptide further comprises a linker region between the first and second amino acid sequences. In some embodiments of any of the embodiments disclosed herein, the polypeptide further comprises an oligopeptide having at least about 90%, at least about 95%, at least about 97%, at least about 98%, at least about 99%, or about 100% sequence identity to the amino acid sequence of SEQ ID NO: 27 or SEQ ID NO: 35 at the C terminus of the first amino acid sequence. In some embodiments of any of the embodiments disclosed herein, the hybrid polypeptide has an amino acid sequence at least about 60%, at least about 65%, at least about 70%, at least about 75%, at least about 80%, at least about 85%, at least about 90%, at least about 95%, at least about 97%, at least about 98%, at least about 99%, or about 100% sequence identity to the amino acid sequence of SEQ ID NO: 75, SEQ ID NO: 76, SEQ ID NO: 77. SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 106, SEQ ID NO: 107, SEQ ID NO: 108, SEQ ID NO: 109, SEQ ID NO: 111, SEQ ID NO: 112, SEQ ID NO: 113, SEQ ID NO: 114, SEQ ID NO: 116, SEQ ID NO: 117, SEQ ID NO: 118, SEQ ID NO: 119, SEQ ID NO: 121, SEQ ID NO: 122, SEQ ID NO: 123, SEQ ID NO: 124, SEQ ID NO: 126, SEQ ID NO: 127, SEQ ID NO: 128, or SEQ ID NO: 129. In some embodiments of any of the embodiments disclosed herein, the first amino acid sequence lacks a CBM. In some embodiments of any of the embodiments disclosed herein, the first amino acid sequence comprises SEQ ID NO:3. In some embodiments, the polypeptide further comprises an amino acid substitution at position 58 of SEQ ID NO:3. In some embodiments, the amino acid substitution is selected from the group consisting of V58P. V58G, V58A, V58L, V58I, V58F. V58Y. V58W, V58S, V66T. V58C. V58M, V58N, V58Q. V58D, V58E, V58K, V58R, and V58H.A polynucleotide encoding any of the hybrid polypeptides disclosed herein.

In other aspects, provided herein is a vector comprising any of the polynucleotides disclosed herein.

In additional aspects, provided herein is a recombinant host cell comprising any of the polynucleotides disclosed herein or any of the vectors disclosed herein. In some embodiments, the host cell is an ethanologenic microorganism. In some embodiments of any of the embodiments disclosed herein, the host cell is a yeast cell. In some embodiments of any of the embodiments disclosed herein, the host cell further expresses and secretes one or more additional enzymes selected from the group consisting of protease, hemicellulase, cellulase, peroxidase, lipolytic enzyme, metallolipolytic enzyme, xylanase, lipase, phospholipase, esterase, perhydrolase, cutinase, pectinase, pectate lyase, mannanase, keratinase, reductase, nuclease, oxidase, phenoloxidase, lipoxygenase, ligninase, alpha-amylase, pullulanase, phytase, tannase, pentosanase, malanase, beta-glucanase, arabinosidase, hyaluronidase, chondroitinase, laccase, transferrase, and a combination thereof.

In yet other aspects, provided herein is a method of producing a hybrid polypeptide in a host cell comprising culturing any of the host cells disclosed herein under conditions suitable for the expression and production of the hybrid polypeptide. In some embodiments, the method further comprises recovering the hybrid polypeptide from the culture.

In another aspect, provided herein is an enzyme composition comprising any of the hybrid polypeptides disclosed herein. In some embodiments, the enzyme composition is used in a starch conversion process.

In still further aspects, provided herein is a method for saccharifying a starch-containing material comprising the steps of: i) contacting the starch-containing material with an alpha-amylase; and ii) contacting the starch-containing material with any of the hybrid polypeptides disclosed herein; wherein the method produces at least 70% free glucose from the starch-containing material (substrate). In some embodiments, the method includes sequentially or simultaneously performing step (i) and step (ii). In some embodiments of any of the embodiments disclosed herein, the method further comprises a pre-saccharification before saccharification step ii). In some embodiments of any of the embodiments disclosed herein, the step (i) is carried out at or below the gelatinization temperature of the starch-containing material.

In another aspect, provided herein is a saccharide produced by any of the methods disclosed herein.

In other aspects, provided herein is a method for producing fermentation products from the saccharides produced by any of the methods disclosed herein, wherein the saccharide is fermented by a fermenting organism. In some embodiments, the fermentation process is performed sequentially or simultaneously with the step (ii). In some embodiments of any of the embodiments disclosed herein, the fermentation product comprises ethanol. In some embodiments of any of the embodiments disclosed herein, the fermentation product comprises a non-ethanol metabolite. In some embodiments, the metabolite is citric acid, lactic acid, succinic acid, monosodium glutamate, gluconic acid, sodium gluconate, calcium gluconate, potassium gluconate, an organic acid, glucono delta-lactone, sodium erythorbate, omega 3 fatty acid, butanol, iso-butanol, an amino acid, lysine, tyrosine, threonine, glycine, itaconic acid, 1,3-propanediol, vitamins, enzymes, hormones, isoprene or other biochemicals or biomaterials.

In still further aspects, provided herein is a method of applying any of the hybrid polypeptides disclosed herein in brewing.

In other aspects provided herein is a method of applying any of the hybrid polypeptides disclosed herein to produce beer or a malt-based beverage.

In yet additional aspects, provided herein is a method for increasing starch digestibility in an animal which comprises adding any of the hybrid polypeptides disclosed herein as a feed additive to feed for an animal.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### DETAILED DESCRIPTION

Zygomycetes, also known as “pin molds,” are fungi belonging to the Eumycota, the true fungi that form extended mycelia and diverse asexual and sexual spore structures. Zygomycetes are fungi that thrive in soil and dead plant material (Dijksterhuis & Samson, “Food Spoilage Microorganisms,” Woodhead Publishing Series in Food Science, Technology and Nutrition 2006, Pages 415-436, incorporated by reference herein). Many glucoamylase polypeptides found in Zygomycetes lack carbohydrate binding domains (CBDs). As will be described further herein, the inventors of the present application have surprisingly discovered that addition of one or more CBDs derived from enzymes found in other organisms to glucoamylase polypeptides derived from Zygomycetes to form chimeric polypeptides, results in higher activity for the hydrolysis of starch compared to Zygomycetes glucoamylase polypeptides lacking CBDs.

### I. Definitions

Prior to describing the compositions and methods in detail, the following terms and abbreviations are defined.

Unless otherwise defined, all technical and scientific terms used have their ordinary meaning in the relevant scientific field. Singleton, et al., Dictionary of Microbiology and Molecular Biology, 2d Ed., John Wiley and Sons, New York (1994), and Hale & Markham, Harper Collins Dictionary of Biology, Harper Perennial, NY (1991) provide the ordinary meaning of many of the terms describing the invention.

The term “glucoamylase (1,4-alpha-D-glucan glucohydrolase, EC 3.2.1.3) activity” is defined herein as an enzyme activity, which catalyzes the release of D-glucose from the non-reducing ends of starch or related oligo- and poly-saccharide molecules.

“Catalytic domain” (CD) or “catalytic module” (CM) as used interchangably herein, refer to a structural region of a polypeptide (such as a glucoamylase) which includes the active site for substrate hydrolysis (such as starch or related oligo- and poly-saccharide molecules).

“Chimeric polypeptide” or “hybrid polypeptide” or “hybrid glucoamylase polypeptide” or “chimeric glucoamylase polypeptide” are used interchangably herein to refer to a polypeptide that includes within it at least two polypeptides (or portions thereof such as subsequences, peptides, or functional domains such as carbohydrate binding modules) from different sources. Chimeric or hybrid polypeptides may include two, three, four, five, six, seven, or more polypeptides or portions thereof from different sources, such as different genes, different cDNAs, or different animal or other species. Chimeric or hybrid polypeptides may include one or more linkers joining the different polypeptides or portions thereof. Thus, the polypeptides or portions thereof may be joined directly or they may be joined indirectly, via linkers, or both, within a single chimeric polypeptide. Chimeric or hybrid polypeptides may include additional peptides such as signal sequences and sequences such as 6His and FLAG that aid in protein purification or detection. In addition, chimeric or hybrid polypeptides may have amino acid or peptide additions to the N- and/or C-termini.

“Zygomycete fungi” refers to a former division or phylum of the kingdom Fungi. The members are now part of two phyla the Mucoromycota and Zoopagomycota with approximately 1060 species known (Spatafora et al., 2016, Mycologia. 108 (5): 1028-1046, incorporated by reference herein). “Mucorales” is the largest and best studied order of Zygomycete fungi. Members of this order are sometimes called pin molds.

The terms “carbohydrate binding domain” (CBD) or “carbohydrate binding module” (CBM) or “starch binding module” (SBM) or “starch binding domain” (SBD), as used interchangably herein, refer to a functional protein domain that is present in carbohydrate-active enzymes (for example endocellulases and exocellulases) and having carbohydrate-binding activity. In some instances, carbohydrate binding domains can contribute to catalytic efficiency or thermostability by, for example, increasing enzyme-substrate complex formations. While CBMs are often naturally occurring within larger enzymes (typically connected via a linker region to one or more catalytic domains), the term as used herein refers to the independent module. A CBM in its naturally occurring form may be located at the N-terminus, C-terminus, or at an internal position of a polypeptide, and as used herein may be a truncation of its naturally occurring form.

The term “linker” as used herein refers to an amino acid sequence functioning to connect separate protein domains with one another.

The term “prelinker” as used herein refers to an amino acid sequence functioning to connect a catalytic domain (such as the catalytic domain of a glucoamylase) and a linker.

The term, “wild-type,” with respect to a polypeptide (such as a glucoamylase), refers to a naturally-occurring polypeptide that does not include a human-made substitution, insertion (such as the insertion of one or more CBMs), or deletion at one or more amino acid positions.

The terms, “parent, “parental,” or “reference” with respect to a polypeptide (such as a glucoamylase), can refer to a wild-type polypeptide or can also refer to a polypeptide that has had one or more amino acid substitutions introduced into it which is then used as a reference to compare performance characteristics of a polypeptide that has had further amino acid substitutions or one or more functional protein domains (such as a CBM) introduced into it. In some embodiments, the parent polypeptide is a glucoamylase derived from Zygomycetes or Mucorales (such as Saksenaea vasiformis). In some embodiments, the parent polypeptide is SEQ ID NOs: 3 or 41.

The term “amino acid sequence” is synonymous with the terms “polypeptide”, “protein” and “peptide” and are used interchangeably. Where such amino acid sequences exhibit activity, they may be referred to as an “enzyme”. The conventional one-letter or three-letter codes for amino acid residues are used, with amino acid sequences being presented in the standard amino-to-carboxy terminal orientation (i.e., N→C).

The term “mature polypeptide” is defined herein as a polypeptide in its final form following translation and any post-translational modifications, such as N-terminal processing, C-terminal truncation, glycosylation, phosphorylation, etc.

A “signal sequence” or “signal peptide” is a sequence of amino acids attached to the N-terminal portion of a protein, which facilitates the secretion of the protein outside the cell. The mature form of an extracellular protein lacks the signal sequence, which is cleaved off during the secretion process. In some embodiments, SEQ ID NOs: 45, 51, 57, 63, or 69 are signal peptides.

The term “nucleic acid” or “polynucleotide” can be used interchangable to encompass DNA, RNA, heteroduplexes, and synthetic molecules capable of encoding a polypeptide. Nucleic acids may be single stranded or double stranded, and may be chemically modified. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

The term “coding sequence” means a polynucleotide sequence, which directly specifies the amino acid sequence of its protein product. The boundaries of the coding sequence are generally determined by an open reading frame, which usually begins with the ATG start codon or alternative start codons such as GTG and TTG and ends with a stop codon such as TAA. TAG, and TGA. The coding sequence may be a DNA, cDNA, synthetic, or recombinant nucleotide sequence.

The term “cDNA” is defined herein as a DNA molecule that can be prepared by reverse transcription from a mature, spliced, mRNA molecule obtained from a eukaryotic cell. cDNA lacks intron sequences that may be present in the corresponding genomic DNA. The initial, primary RNA transcript is a precursor to mRNA that is processed through a series of steps before appearing as mature spliced mRNA. These steps include the removal of intron sequences by a process called splicing. cDNA derived from mRNA lacks, therefore, any intron sequences.

A “host strain” or “host cell” is an organism into which an expression vector, phage, virus, or other DNA construct, including a polynucleotide encoding a polypeptide of interest (e.g., a hybrid glucoamylase) has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

The term “expression” refers to the process by which a polypeptide is produced based on a nucleic acid sequence. The process includes both transcription and translation.

The term “vector” refers to a polynucleotide sequence designed to introduce nucleic acids into one or more cell types. Vectors include cloning vectors, expression vectors, shuttle vectors, plasmids, phage particles, cassettes and the like.

An “expression vector” refers to a DNA construct comprising a DNA sequence encoding a polypeptide of interest, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

The term “control sequences” is defined herein to include all components necessary for the expression of a polynucleotide encoding a polypeptide of the present invention. Each control sequence may be native or foreign to the nucleotide sequence encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequences.

“Biologically active” refer to a sequence having a specified biological activity, such an enzymatic activity.

The term “specific activity” refers to the number of moles of substrate that can be converted to product by an enzyme or enzyme preparation per unit time under specific conditions. Specific activity is generally expressed as units (U)/mg of protein.

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions X 100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268. modified as in Karlin and Altschul, 1993. Proc. Natl. Acad. Sci. U.S.A. 90:5873-5877. Such an algorithm is incorporated into the NBLAST and XBLAST programs of Altschul et al., 1990. J. Mol. Biol. 215:403. BLAST nucleotide searches can be performed with the NBLAST nucleotide program parameters set, e.g., for score=100, word length=12 to obtain nucleotide sequences homologous to a nucleic acid molecules of the present application. BLAST protein searches can be performed with the XBLAST program parameters set, e.g., to score-50, word length=3 to obtain amino acid sequences homologous to a protein molecule described herein. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (ld.). When utilizing BLAST, Gapped BLAST, and PSI-Blast programs, the default parameters of the respective programs (e.g., of XBLAST and NBLAST) can be used (see, e.g., the NCBI website). Another preferred non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Another computer program that can be used to create multiple alignments of protein sequences is MUSCLE. Elements of the MUSCLE algorithm include fast distance estimation using kmer counting, progressive alignment using a new profile function described as log-expectation score, and refinement using tree-dependent restricted partitioning. This program is described in MUSCLE: multiple sequence alignment with high accuracy and high throughput by Robert C. Edgar (2004) published in Nucleic Acids Res. 32:1792-1797. The percent identity between two sequences can be determined using techniques similar to those described above, with or without allowing gaps. In calculating percent identity, typically only exact matches are counted.

The term “homologous sequence” is defined herein as a predicted protein having an E value (or expectancy score) of less than 0.001 in a tfasty search (Pearson, W. R., 1999, in Bioinformatics Methods and Protocols, S. Misener and S. A. Krawetz, ed., pp. 185-219) with a glucoamylase, for example, the glucoamylase of SEQ ID NOs: 3 or 41.

As used herein, an “equivalent position” or “corresponding position” means a position that is common to two amino acid sequences that is based on an alignment of the amino acid sequence of a parent glucoamylase with a glucoamylase variant as well as alignment of a three-dimensional structure of a parent glucoamylase with that of a variant glucoamylase in three-dimensional space.

As used herein with regard to amino acid residue positions, “corresponding to” or “corresponds to” or “correspond to” or “corresponds” refers to an amino acid residue at the enumerated position in a protein or peptide, or an amino acid residue that is analogous, homologous, or equivalent to an enumerated residue in a protein or peptide. As used herein, “corresponding region” generally refers to an analogous position in a related protein or a reference protein.

As used herein, “performance index” or “PI” refers to calculated activity per unit of an enzyme relative to a parent molecule. In some aspects of any of the embodiments disclosed herein, the parental molecule used in the calculation of the performance index is a glucoamylase. In some embodiments, the parental molecule has a performance index of one, by definition. In other embodiments, a performance index greater than one (PI>1.0) indicates improved activity of a glucoamylase variant compared to the parent molecule.

The phrase “simultaneous saccharification and fermentation (SSF)” refers to a process in the production of biochemicals in which a microbial organism, such as an ethanologenic microorganism, and at least one enzyme, such as an amylase, are present during the same process step. SSF includes the contemporaneous hydrolysis of starch substrates (granular, liquefied, or solubilized) to saccharides, including glucose, and the fermentation of the saccharides into alcohol or other biochemical or biomaterial in the same reactor vessel.

A “slurry” is an aqueous mixture containing insoluble starch granules in water.

The term “total sugar content” refers to the total soluble sugar content present in a starch composition including monosaccharides, oligosaccharides and polysaccharides.

The term “dry solids” (ds) refer to dry solids dissolved in water, dry solids dispersed in water or a combination of both. Dry solids thus include granular starch, and its hydrolysis products, including glucose.

The term “high DS” refers to aqueous starch slurry with a dry solid content greater than 38% (wt/wt).

“Degree of polymerization (DP)” refers to the number (n) of anhydroglucopyranose units in a given saccharide. Examples of DP1 are monosaccharides, such as glucose and fructose. Examples of DP2 are disaccharides, such as maltose and sucrose. A DP4+ (>DP3) denotes polymers with a degree of polymerization of greater than 3.

The term “contacting” refers to the placing of referenced components (including but not limited to enzymes, substrates, and fermenting organisms) in sufficiently close proximity to affect an expect result, such as the enzyme acting on the substrate or the fermenting organism fermenting a substrate.

As used herein, the terms “yeast cells,” “yeast strains,” or simply “yeast” refer to organisms from the Ascomycota and Basidiomycota. Exemplary yeast is budding yeast from the order Saccharomyces. Particular examples of yeast are Saccharomyces spp., including but not limited to S. cerevisiae. Yeast include organisms used for the production of fuel alcohol as well as organisms used for the production of potable alcohol, including specialty and proprietary yeast strains used to make distinctive-tasting beers, wines, and other fermented beverages.

An “ethanologenic microorganism” refers to a microorganism with the ability to convert a sugar or other carbohydrates to ethanol.

The term “biochemicals” refers to a metabolite of a microorganism, such as citric acid, lactic acid, succinic acid, monosodium glutamate, gluconic acid, sodium gluconate, calcium gluconate, potassium gluconate, glucono delta-lactone, sodium erythorbate, omega 3 fatty acid, butanol, iso-butanol, an amino acid, lysine, itaconic acid, other organic acids, 1,3-propanediol, vitamins, or isoprene or other biomaterial.

The term “pullulanase” also called debranching enzyme (E.C. 3.2.1.41, pullulan 6-glucanohydrolase), is capable of hydrolyzing alpha 1-6 glucosidic linkages in an amylopectin molecule.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −15% to +15% of the numerical value, unless the term is otherwise specifically defined in context.

As used herein, the singular terms “a,” “an,” and “the” include the plural reference unless the context clearly indicates otherwise.

It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

The term “comprising”, and its cognates are used in their inclusive sense; that is, equivalent to the term “including” and its corresponding cognates. It is further noted that the term “comprising,” as used herein, means including, but not limited to, the component(s) after the term “comprising.” The component(s) after the term “comprising” are required or mandatory, but the composition comprising the component(s) can further include other non-mandatory or optional component(s).

It is also noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is also noted that the term “consisting of,” as used herein, means including, and limited to, the component(s) after the term “consisting of.” The component(s) after the term “consisting of” are therefore required or mandatory, and no other component(s) are present in the composition.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless defined otherwise herein, all technical and scientific terms used herein have the same meaning as commonly understood by one of ordinary skill in the art to which this invention pertains.

Other definitions of terms may appear throughout the specification.

### II. Compositions

Provided herein are hybrid (a.k.a. chimeric) polypeptides comprising a first amino acid sequence containing a catalytic module (CM) with glucoamylase activity or a functional fragment thereof; and a second amino acid sequence with a carbohydrate-binding module (CBM) or a functional fragment thereof.

### A. Catalytic Modules

In a first aspect, the present invention relates to hybrid polypeptides having a catalytic module (CM) or functional fragment thereof having glucoamylase activity (i.e. the ability to catalyze the release of D-glucose from the non-reducing ends of starch or related oligo- and poly-saccharide molecules) derived from Zygomycetes (for example, Mucorales). Nonlimiting examples of species found within Zygomycetes from which glucoamylase CMs can be derived include those from Saksenaea vasiformis, Saksenaea oblongispora, Apophysomyces ossiformis, Apophysomyces elegans, Apophysomyces variabilis, and Apophysomyces trapeziformis.

Nonlimiting examples of CMs having glucoamylase activity derived from Zygomycetes include those having an amino acid sequence sharing at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, and at least about 99%, or 100% amino acid sequence identity to the polypeptide of SEQ ID NOs: 3, 33, 41, 47, 49, 53, 55, 59, 61, 65, 67, 71, 73, 105, 110, 115, 120, or 125.

In some emobdiments, the CMs are separated from a CBM by a linker peptide sequence. Non-limting examples of linker peptides include an oligopeptide having at least about 90%, at least about 95%, at least about 97%, at least about 98%, at least about 99%, or about 100% sequence identity to the amino acid sequence of SEQ ID NO:29 (from Aspergillus niger) or an oligopeptide having at least about 90%, at least about 95%, at least about 97%, at least about 98%, at least about 99%, or about 100% sequence identity to the amino acid sequence of SEQ ID NO:37 (from Trichoderma reesei). In some embodiments, the linker oligopeptide is located at the C-terminus of the CM. In further embodiments, the linker oligopeptide is located at the N-terminus of the CM.

In further embodiments, a prelinker peptide sequence can separate the CD from the linker. Non-limting examples of pre-linker peptides include an oligopeptide having at least about 90%, at least about 95%, at least about 97%, at least about 98%, at least about 99%, or about 100% sequence identity to the amino acid sequence of SEQ ID NO: 27 (from Aspergillus niger) or an oligopeptide having at least about 90%, at least about 95%, at least about 97%, at least about 98%, at least about 99%, or about 100% sequence identity to the amino acid sequence of SEQ ID NO:35 (from Trichoderma reesei). In some embodiments, the pre-linker oligopeptide is located at the C-terminus of the CM. In further embodiments, the linker oligopeptide is located at the N-terminus of the CM.

In some embodiments, the CMs having glucoamylase activity or functional fragments thereof are variant polypeptides comprising amino acid sequences that differ by no more than ten amino acids, no more than nine amino acids, no more than eight amino acids, no more than seven amino acids, no more than six amino acids no more than five amino acids, no more than four amino acids, no more than three amino acids, no more than two amino acids, and even no more than one amino acid from the polypeptides of SEQ ID NOs: 3, 33, 41, 47, 49, 53, 55, 59, 61, 65, 67, 71, 73, 105, 110, 115, 120, or 125.

In some embodiments, the CMs having glucoamylase activity or functional fragment thereof for use in the present invention additionally have one or more of pullulan and/or panose and/or maltodextrin hydrolyzing activity. In a further embodiment, the wildtype CM having glucoamylase activity or functional fragment thereof for use in the present invention lacks an amino acid sequence that encodes a CBM or a functional CBM.

### B. Carbohydrate Binding Modules (CBMs)

Carbohydrate-binding modules (CBMs), previously known as cellulose-binding domains, are protein domains found in carbohydrate-active enzymes (for example, glycoside hydrolases). The majority of these domains have carbohydrate-binding activity. CBMs are classified into numerous families, based on amino acid sequence similarity. There are currently (December 2021) 88 families of CBM in the CAZy database.

Exemplary CBM families for use in any of the hybrid polypeptides disclosed herein include those of CBM families 20, 21, 25, 26, 34, 41, and 45. With reference to cazy.org/Carbohydrate-Binding-Modules, CBM Family 20 includes modules that bind to starch and cyclodextrins. The granular starch-binding function has been demonstrated in several cases. CBM family 21 includes modules of approx. 100 residues and is found in many eukaryotic proteins involved in glycogen metabolism. The granular starch-binding function has been demonstrated in one case. CBM family 25 binds to alpha-glucooligosaccharides, particularly those containing alpha-1,6 linkages, and granular starch. Regarding CBM family 26, the starch-binding function has been demonstrated in two cases. This module was formerly known as X22 modules and are structurally related to CBM25 modules. For CBM Family 34, modules of approx. 120 residues have been shown as well as granular starch-binding function demonstrated in the case of Thermoactinomyces vulgaris R-47 α-amylase 1 (TVAI). CBM Family 41 includes modules of approx. 100 residues found in primarily in bacterial pullulanases. The N-terminal module from Thermotoga maritima Pul13 has been shown to bind to the α-glucans amylose, amylopectin, pullulan, and oligosaccharide fragments derived from these polysaccharides. The CBM45 has modules of approx. 100 residues and has been found at the N-terminus of plastidial α-amylases and of α-glucan, water dikinases. Starch-binding activity has been demonstrated in the case of potato α-glucan, water dikinase.

In some embodiments, the CBM or functional fragment thereof has at least about 70%, at least about 75%, at least about 80%, at least about 85%, at least about 90%, at least about 95%, at least about 97%, at least about 98%, at least about 99%, or about 100% sequence identity to amino acids 25-130 of SEQ ID NO: 1. In other embodiments, the CBM has at least about 70%, at least about 75%, at least about 80%, at least about 85%, at least about 90%, at least about 95%, at least about 97%, at least about 98%, at least about 99%, or about 100% sequence identity to amino acids 24-130 of SEQ ID NO: 5. In other embodiments, the CBM has at least about 70%, at least about 75%, at least about 80%, at least about 85%, at least about 90%, at least about 95%, at least about 97%, at least about 98%, at least about 99%, or about 100% sequence identity to amino acids 22-130 of SEQ ID NO: 7. In other embodiments, the CBM has at least about 70%, at least about 75%, at least about 80%, at least about 85%, at least about 90%, at least about 95%, at least about 97%, at least about 98%, at least about 99%, or about 100% sequence identity to amino acids 24-130 of SEQ ID NO: 9. In other embodiments, the CBM has at least about 70%, at least about 75%, at least about 80%, at least about 85%, at least about 90%, at least about 95%, at least about 97%, at least about 98%, at least about 99%, or about 100% sequence identity to amino acids 24-130 of SEQ ID NO: 11. In other embodiments, the CBM has at least about 70%, at least about 75%, at least about 80%, at least about 85%, at least about 90%, at least about 95%, at least about 97%, at least about 98%, at least about 99%, or about 100% sequence identity to amino acids 22-130 of SEQ ID NO: 13. In other embodiments, the CBM has at least about 70%, at least about 75%, at least about 80%, at least about 85%, at least about 90%, at least about 95%, at least about 97%, at least about 98%, at least about 99%, or about 100% sequence identity to amino acids 25-130 of SEQ ID NO: 15. In other embodiments, the CBM has at least about 70%, at least about 75%, at least about 80%, at least about 85%, at least about 90%, at least about 95%, at least about 97%, at least about 98%, at least about 99%, or about 100% sequence identity to amino acids 26-131 of SEQ ID NO: 17. In other embodiments, the CBM has at least about 70%, at least about 75%, at least about 80%, at least about 85%, at least about 90%, at least about 95%, at least about 97%, at least about 98%, at least about 99%, or about 100% sequence identity to amino acids 25-130 of SEQ ID NO: 19. In other embodiments, the CBM has at least about 70%, at least about 75%, at least about 80%, at least about 85%, at least about 90%, at least about 95%, at least about 97%, at least about 98%, at least about 99%, or about 100% sequence identity to amino acids 24-127 of SEQ ID NO: 21. In other embodiments, the CBM has at least about 70%, at least about 75%, at least about 80%, at least about 85%, at least about 90%, at least about 95%, at least about 97%, at least about 98%, at least about 99%, or about 100% sequence identity to amino acids 23-145 of SEQ ID NO: 23. In other embodiments, the CBM has at least about 70%, at least about 75%, at least about 80%, at least about 85%, at least about 90%, at least about 95%, at least about 97%, at least about 98%, at least about 99%, or about 100% sequence identity to amino acids 28-131 of SEQ ID NO: 25. In other embodiments, the CBM has at least about 70%, at least about 75%, at least about 80%, at least about 85%, at least about 90%, at least about 95%, at least about 97%, at least about 98%, at least about 99%, or about 100% sequence identity SEQ ID NO: 31. In other embodiments, the CBM has at least about 70%, at least about 75%, at least about 80%, at least about 85%, at least about 90%, at least about 95%, at least about 97%, at least about 98%, at least about 99%, or about 100% sequence identity SEQ ID NO: 39.

In some embodiments, the CBMs are variant polypeptides comprising amino acid sequences that differ by no more than ten amino acids, no more than nine amino acids, no more than eight amino acids, no more than seven amino acids, no more than six amino acids no more than five amino acids, no more than four amino acids, no more than three amino acids, no more than two amino acids, and even no more than one amino acid from amino acids 25-130 of SEQ ID NO: 1, amino acids 24-130 of SEQ ID NO: 5, amino acids 22-130 of SEQ ID NO: 7, amino acids 24-130 of SEQ ID NO: 9, amino acids 24-130 of SEQ ID NO: 11, amino acids 22-130 of SEQ ID NO: 13, amino acids 25-130 of SEQ ID NO: 15, amino acids 26-131 of SEQ ID NO: 17, amino acids 25-130 of SEQ ID NO: 19, amino acids 24-127 of SEQ ID NO: 21, amino acids 23-145 of SEQ ID NO: 23, amino acids 28-131 of SEQ ID NO: 25, SEQ ID NO: 31 or SEQ ID NO: 39.

The CBM or functional fragment thereof, in some non-limiting embodiments, can also include a signal peptide. Suitable examples of signal peptides include the amino acid sequences of SEQ ID NOs: 45, 51, 57, 63, or 69.

### C. Chimeric Polypeptides

Provided herein are hybrid polypeptides comprising any of the catalytic modules having glucoamylase activity derived from Zygomycetes (e.g., Mucorales) or functional fragment thereof disclosed herein and one or more (such as any of 1, 2, 3, or 4) carbohydrate-binding modules or functional fragments thereof (such as any of the CBMs disclosed herein).

The hybrid polypeptides can include any of those disclosed herein, including those having an amino acid sequence at least about 60%, at least about 65%, at least about 70%, at least about 75%, at least about 80%, at least about 85%, at least about 90%, at least about 95%, at least about 97%, at least about 98%, at least about 99%, or about 100% identical to the amino acid sequence of any of SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89. SEQ ID NO: 106, SEQ ID NO: 107, SEQ ID NO: 108, SEQ ID NO: 109, SEQ ID NO: 111, SEQ ID NO: 112, SEQ ID NO: 113, SEQ ID NO: 114, SEQ ID NO: 116, SEQ ID NO: 117, SEQ ID NO: 118, SEQ ID NO: 119, SEQ ID NO: 121, SEQ ID NO: 122, SEQ ID NO: 123, SEQ ID NO: 124, SEQ ID NO: 126, SEQ ID NO: 127, SEQ ID NO: 128, or SEQ ID NO: 129.

In some embodiments, the hybrid polypeptides disclosed herein (such as any of SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 106, SEQ ID NO: 107, SEQ ID NO: 108, SEQ ID NO: 109, SEQ ID NO: 111, SEQ ID NO: 112, SEQ ID NO: 113, SEQ ID NO: 114, SEQ ID NO: 116, SEQ ID NO: 117, SEQ ID NO: 118, SEQ ID NO: 119, SEQ ID NO: 121, SEQ ID NO: 122, SEQ ID NO: 123, SEQ ID NO: 124, SEQ ID NO: 126, SEQ ID NO: 127, SEQ ID NO: 128, or SEQ ID NO: 129) exhibit improved starch hydrolyzing activity (such as any of about 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 180%, 185%, 190%, 195%, 200%, or more, inclusive of values falling in between these percentages improved starch hydrolyzing activity) compared to a parent glucoamylase polypeptide derived from Zygomycetes (e.g., Mucorales) that has a CD but which lacks a CBD. Starch hydrolyzing activity can be determined by any way known in the art, including those shown herein in Examples 3 and 5.

In other embodiments, the hybrid polypeptides disclosed herein (such as any of SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 106, SEQ ID NO: 107, SEQ ID NO: 108, SEQ ID NO: 109, SEQ ID NO: 111, SEQ ID NO: 112, SEQ ID NO: 113, SEQ ID NO: 114, SEQ ID NO: 116, SEQ ID NO: 117, SEQ ID NO: 118, SEQ ID NO: 119, SEQ ID NO: 121, SEQ ID NO: 122, SEQ ID NO: 123, SEQ ID NO: 124, SEQ ID NO: 126, SEQ ID NO: 127, SEQ ID NO: 128, or SEQ ID NO: 129) exhibit improved thermostability (such as any of about 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 180%, 185%, 190%, 195%, 200%, or more, inclusive of values falling in between these percentages improved thermostability) compared to a parent glucoamylase polypeptide derived from Zygomycetes (e.g., Mucorales) that has a CD but which lacks a CBD. Thermostability of polypeptides can be determined by any way known in the art.

In another aspect, the hybrid polypeptides disclosed herein can, in some embodiments, comprise conservative substitution(s) of one or several amino acid residues relative to the amino acid sequence SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 106, SEQ ID NO: 107, SEQ ID NO: 108, SEQ ID NO: 109, SEQ ID NO: 111, SEQ ID NO: 112, SEQ ID NO: 113, SEQ ID NO: 114, SEQ ID NO: 116, SEQ ID NO: 117, SEQ ID NO: 118, SEQ ID NO: 119, SEQ ID NO: 121, SEQ ID NO: 122, SEQ ID NO: 123, SEQ ID NO: 124, SEQ ID NO: 126, SEQ ID NO: 127, SEQ ID NO: 128, or SEQ ID NO: 129.

Exemplary conservative amino acid substitutions are listed below. Some conservative substitutions (i.e., mutations) can be produced by genetic manipulation while others are produced by introducing synthetic amino acids into a polypeptide by other means.

| For Amino Acid | Code | Replace with any of |
|---|---|---|
| Alanine | A | D-Ala, Gly, beta-Ala, L-Cys, D-Cys |
| Arginine | R | D-Arg, Lys, D-Lys, homo-Arg, D-homo-Arg, Met, |
| | | Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, b-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, Met, |
| | | D-Met, Ile, D-Ile, Orn, D-Orn |
| Methionine | M | D-Met, S-Me-Cys, Ile, D-Ile, Leu, D-Leu, Val, D-Val |
| Phenylalanine | F | D-Phe, Tyr, D-Thr, L-Dopa, His, D-His, Trp, D-Trp, |
| | | Trans-3,4, or 5-phenylproline, cis-3,4, or 5-phenylproline |
| Proline | P | D-Pro, L-I-thioazolidine-4- carboxylic acid, D-or |
| | | L-1-oxazolidine-4-carboxylic acid |
| Serine | S | D-Ser, Thr, D-Thr, allo-Thr, Met, D-Met, Met(O), D-Met(O), |
| | | L-Cys, D-Cys |
| Threonine | T | D-Thr, Ser, D-Ser, allo-Thr, Met, |
| | | D-Met, Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Additionally, where the CD comprises the amino acid sequence of SEQ ID NO: 3 (or any amino acid sequence at least about 60%, at least about 65%, at least about 70%, at least about 75%, at least about 80%, at least about 85%, at least about 90%, at least about 95%, at least about 97%, at least about 98%, at least about 99%, or about 100% identical to the amino acid sequence of SEQ ID NO: 3), the CD may further comprise an additional amino acid substitution at position 58. In some embodiments, the subsition comprises any of V58P, V58G, V58A, V58L, V58I, V58F, V58Y, V58W, V58S, V66T, V58C, V58M, V58N, V58Q, V58D, V58E, V58K, V58R, or V58H.

### D. Enzyme Compositions

The present invention also relates to compositions comprising a hybrid polypeptide (such as a hybrid glucoamylase polypeptide, for example, any of the hybrid glucoamylase polypeptides disclosed herein) and/or a starch substrate. In some embodiments, a hybrid polypeptide comprising an amino acid sequence that is at least about 60%, at least about 65%, at least about 70%, at least about 75%, at least about 80%, at least about 85%, at least about 90%, at least about 95%, at least about 96%, at least about 97%, at least about 98%, at least about 99%, identical to that of SEQ ID NOs: 75-89 and 106-129 can also be used in the enzyme composition. Preferably, the compositions are formulated to provide desirable characteristics such as low color, low odor and acceptable storage stability at a temperature of about 4-40° C. and a pH of about 3-7.

The composition may comprise a hybrid glucoamylase polypeptide of the present invention as the major enzymatic component. Alternatively, the composition may comprise multiple enzymatic activities, such as an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, alpha-glucosidase, beta-glucosidase, beta-amylase, isoamylase, haloperoxidase, invertase, laccase, lipase, mannosidase, oxidase, pectinolytic enzyme, peptidoglutaminase, peroxidase, phytase, polyphenoloxidase, proteolytic enzyme, pullulanase, ribonuclease, transglutaminase, xylanase or a combination thereof, which may be added in effective amounts well known to the person skilled in the art.

The polypeptide compositions may be prepared in accordance with methods known in the art and may be in the form of a liquid or a dry composition. For instance, the compositions comprising the present hybrid glucoamylase polypeptides may be aqueous or non-aqueous formulations, granules, powders, gels, slurries, pastes, etc., which may further comprise any one or more of the additional enzymes listed, herein, along with buffers, salts, preservatives, water, co-solvents, surfactants, and the like. Such compositions may work in combination with endogenous enzymes or other ingredients already present in a slurry, water bath, washing machine, food or drink product, etc., for example, endogenous plant (including algal) enzymes, residual enzymes from a prior processing step, and the like. The polypeptide to be included in the composition may be stabilized in accordance with methods known in the art.

The composition may be cells expressing the polypeptide, including cells capable of producing a product from fermentation. Such cells may be provided in a liquid or in dry form along with suitable stabilizers. Such cells may further express additional polypeptides, such as those mentioned, above.

The dosage of the hybrid glucoamylase polypeptide composition of the invention and other conditions under which the composition is used may be determined on the basis of methods known in the art.

The above composition is suitable for use in liquefaction, saccharification, and/or fermentation process, preferably in starch conversion, especially for producing syrup and fermentation products, such as ethanol. The composition is also suitable for use in animal nutrition (such as a component of an animal feed) and fermented beverage production.

### III. Methods

A. Production of hybrid polypeptides

The hybrid glucoamylase polypeptides disclosed herein can be produced in host cells, for example, by secretion or intracellular expression. A cultured cell material (e.g., a whole-cell broth) comprising a hybrid glucoamylase polypeptide can be obtained following secretion of the hybrid glucoamylase polypeptide into the cell medium. Optionally, the hybrid glucoamylase polypeptide can be isolated from the host cells, or even isolated from the cell broth, depending on the desired purity of the final glucoamylase. A gene encoding a hybrid glucoamylase polypeptide can be cloned and expressed according to methods well known in the art.

Suitable host cells include bacterial, fungal (including yeast and filamentous fungi), and plant cells (including algae). Particularly useful host cells include Aspergillus spp. (such as, without limitation, Aspergillus niger or Aspergillus oryzae) a Trichoderma spp. (such as, Trichoderma reesei) or a Myceliopthora spp. (such as Myceliopthora thermophila). Other host cells include bacterial cells, e.g., Bacillus spp. (such as, Bacillus subtilis or B. licheniformis), as well as Streptomyces spp. A suitable yeast host organism can be selected from Schizosaccharomyces species or a species of Saccharomyces, including Saccharomyces cerevisiae or a species belonging to Schizosaccharomyces such as, for example, S. pombe species. A strain of the methylotrophic yeast species, Pichia pastoris, can be used as the host organism.

Additionally, the host may express one or more accessory enzymes, proteins, peptides. These may benefit liquefaction, saccharification, fermentation, SSF, and downstream processes. Furthermore, the host cell may produce ethanol and other biochemicals or biomaterials in addition to enzymes used to digest the various feedstock(s). Such host cells may be useful for fermentation or simultaneous saccharification and fermentation processes to reduce or eliminate the need to add enzymes.

### 1. Vectors

A DNA construct comprising a nucleic acid encoding a hybrid glucoamylase polypeptide disclosed herein can be constructed such that it is suitable to be expressed in a host cell. Because of the known degeneracy in the genetic code, different polynucleotides that encode an identical amino acid sequence can be designed and made with routine skill. It is also known that, depending on the desired host cells, codon optimization may be required prior to attempting expression.

A polynucleotide encoding a hybrid glucoamylase polypeptide of the present disclosure can be incorporated into a vector. Vectors can be transferred to a host cell using known transformation techniques, such as those disclosed below.

A suitable vector may be one that can be transformed into and/or replicated within a host cell. For example, a vector comprising a nucleic acid encoding a hybrid glucoamylase polypeptide disclosed herein can be transformed and/or replicated in a bacterial host cell as a means of propagating and amplifying the vector. The vector may also be suitably transformed into an expression host, such that the encoding polynucleotide is expressed as a functional glucoamylase enzyme.

A non-limiting representative useful vector is pTrex3gM (see, Published U.S. patent application No. 20130323798) and pTTT (see, Published U.S. patent application No. 20110020899), which can be inserted into genome of host. The vectors pTrex3gM and pTTT can both be modified with routine skill such that they comprise and express a polynucleotide encoding a variant glucoamylase polypeptide of the invention.

An expression vector normally comprises control nucleotide sequences such as a promoter, operator, ribosome binding site, translation initiation signal and optionally, a repressor gene or one or more activator genes. Additionally, the expression vector may comprise a sequence coding for an amino acid sequence capable of targeting the hybrid glucoamylase polypeptide to a host cell organelle such as a peroxisome, or to a particular host cell compartment. For expression under the direction of control sequences, the nucleic acid sequence of the hybrid glucoamylase polypeptide is operably linked to the control sequences in proper manner with respect to expression.

A polynucleotide encoding a hybrid glucoamylase polypeptide disclosed herein can be operably linked to a promoter, which allows transcription in the host cell. The promoter may be any DNA sequence that shows transcriptional activity in the host cell of choice and may be derived from genes encoding proteins either homologous or heterologous to the host cell. Examples of promoters for directing the transcription of the DNA sequence encoding a glucoamylase, especially in a bacterial host, include the promoter of the lac operon of E. coli, the Streptomyces coelicolor agarase gene dagA or celA promoters, the promoters of the Bacillus licheniformis amylase gene (amyL), the promoters of the Bacillus stearothermophilus maltogenic amylase gene (amyM), the promoters of the Bacillus amyloliquefaciens amylase (amyQ), the promoters of the Bacillus subtilis xylA and xylB genes, and the like.

For transcription in a fungal host, examples of useful promoters include those derived from the gene encoding Aspergillus oryzae TAKA amylase, Rhizomucor miehei aspartic proteinase, Aspergillus niger neutral α-amylase, Aspergillus niger acid stable α-amylase, Aspergillus niger glucoamylase, Rhizomucor miehei lipase, Aspergillus oryzae alkaline protease, Aspergillus oryzae triose phosphate isomerase, Aspergillus nidulans acetamidase and the like. When a gene encoding a hybrid glucoamylase polypeptide is expressed in a bacterial species such as an E. coli, a suitable promoter can be selected, for example, from a bacteriophage promoter including a T7 promoter and a phage lambda promoter. Along these lines, examples of suitable promoters for the expression in a yeast species include but are not limited to the Gal 1 and Gal 10 promoters of Saccharomyces cerevisiae and the Pichia pastoris AOX1 or AOX2 promoters. Expression in filamentous fungal host cells often involves cbh1, which is an endogenous, inducible promoter from T. reesei. See Liu et al. (2008) Acta Biochim. Biophys. Sin (Shanghai) 40 (2): 158-65.

The coding sequence can be operably linked to a signal sequence. The DNA encoding the signal sequence may be a DNA sequence naturally associated with the hybrid glucoamylase polypeptide gene of interest to be expressed, or may be from a different genus or species from which a portion of the hybrid glucoamylase is derived (e.g., the species from which the CM of CBM was derived). A signal sequence and a promoter sequence comprising a DNA construct or vector can be introduced into a fungal host cell and can be derived from the same source. For example, the signal sequence may be the Trichoderma reesei cbh1 signal sequence, which is operably linked to a cbh1 promoter.

An expression vector may also comprise a suitable transcription terminator and, in eukaryotes, polyadenylation sequences operably linked to the DNA sequence encoding a glucoamylase. Termination and polyadenylation sequences may suitably be derived from the same sources as the promoter.

The vector may also comprise a selectable marker, e.g., a gene the product of which complements a defect in the isolated host cell, such as the dal genes from B. subtilis or B. licheniformis, or a gene that confers antibiotic resistance such as, e.g., ampicillin, kanamycin, chloramphenicol or tetracycline resistance. Furthermore, the vector may comprise Aspergillus selection markers such as amdS, argB, niaD and xxsC, a marker giving rise to hygromycin resistance, or the selection may be accomplished by co-transformation, such as known in the art. See e.g., Published International PCT Application WO 91/17243.

### 2. Transformation and Culture of Host Cells

An isolated cell, either comprising a DNA construct or an expression vector, is advantageously used as a host cell in the recombinant production of a hybrid glucoamylase polypeptide. The cell may be transformed with the DNA construct encoding the enzyme, conveniently by integrating the DNA construct (in one or more copies) in the host chromosome. This integration is generally considered to be an advantage, as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector in connection with the different types of host cells.

Examples of suitable bacterial host organisms are Gram positive bacterial species such as Bacillaceae including Bacillus subtilis, Bacillus licheniformis, Bacillus lentus, Bacillus brevis, Geobacillus (formerly Bacillus) stearothermophilus, Bacillus alkalophilus, Bacillus amyloliquefaciens, Bacillus coagulans, Bacillus lautus, Bacillus megaterium, and Bacillus thuringiensis; Streptomyces species such as Streptomyces murinus; lactic acid bacterial species including Lactococcus sp. such as Lactococcus lactis; Lactobacillus sp. including Lactobacillus reuteri; Leuconostoc sp.; Pediococcus sp.; and Streptococcus sp. Alternatively, strains of a Gram negative bacterial species belonging to Enterobacteriaceae including E. coli, or to Pseudomonadaceae can be selected as the host organism.

A suitable yeast host organism can be selected from the biotechnologically relevant yeasts species such as but not limited to yeast species such as Pichia sp., Hansenula sp., or Kluyveromyces, Yarrowinia, Schizosaccharomyces species or a species of Saccharomyces, including Saccharomyces cerevisiae or a species belonging to Schizosaccharomyces such as, for example, S. pombe species. A strain of the methylotrophic yeast species, Pichia pastoris, can be used as the host organism. Alternatively, the host organism can be a Hansenula species.

Suitable host organisms among filamentous fungi include species of Aspergillus, e.g., Aspergillus niger, Aspergillus oryzae, Aspergillus tubigensis, Aspergillus awamori, or Aspergillus nidulans. Alternatively, strains of a Fusarium species, e.g., Fusarium oxysporum or of a Rhizomucor species such as Rhizomucor miehei can be used as the host organism. Other suitable strains include Thermomyces and Mucor species. In addition, Trichoderma sp. can be used as a host. A hybrid glucoamylase polypeptide expressed by a fungal host cell can be glycosylated, i.e., will comprise a glycosyl moiety. The glycosylation pattern can be the same or different as present in the wild-type glucoamylase. The type and/or degree of glycosylation may impart changes in enzymatic and/or biochemical properties.

It is advantageous to delete genes from expression hosts, where the gene deficiency can be cured by the transformed expression vector. Known methods may be used to obtain a fungal host cell having one or more inactivated genes. Any gene from a Trichoderma sp. or other filamentous fungal host that has been cloned can be deleted, for example, cbh1, cbh2, egl1, and egl2 genes. Gene deletion may be accomplished by inserting a form of the desired gene to be inactivated into a plasmid by methods known in the art.

General transformation techniques are known in the art. See, e.g., Sambrook et al. (2001), supra. The expression of heterologous protein in Trichoderma is described, for example, in U.S. Pat. No. 6,022,725. Reference is also made to Cao et al. (2000) Science 9:991-1001 for transformation of Aspergillus strains. Genetically stable transformants can be constructed with vector systems whereby the nucleic acid encoding a glucoamylase is stably integrated into a host cell chromosome. Transformants are then selected and purified by known techniques.

### 3. Expression and Fermentation

A method of producing any of the hybrid glucoamylase polypeptides disclosed herein may comprise cultivating a host cell under conditions conducive to the production of the enzyme and recovering the enzyme from the cells and/or culture medium.

The medium used to cultivate the cells may be any conventional medium suitable for growing the host cell and obtaining expression of a variant glucoamylase polypeptide. Suitable media and media components are available from commercial suppliers or may be prepared according to published recipes (e.g., as described in catalogues of the American Type Culture Collection).

Any of the fermentation methods well known in the art can suitably used to ferment the transformed or the derivative fungal strain as described above. In some embodiments, fungal cells are grown under batch or continuous fermentation conditions.

### 4. Methods for Enriching and Purification

Separation and concentration techniques are known in the art and conventional methods can be used to prepare a concentrated solution or broth comprising a hybrid glucoamylase polypeptide of the invention.

After fermentation, a fermentation broth is obtained, the microbial cells and various suspended solids, including residual raw fermentation materials, are removed by conventional separation techniques in order to obtain a glucoamylase solution. Filtration, centrifugation, microfiltration, rotary vacuum drum filtration, ultrafiltration, centrifugation followed by ultra-filtration, extraction, or chromatography, or the like, are generally used.

It may at times be desirable to concentrate a solution or broth comprising a hybrid glucoamylase polypeptide to optimize recovery. Use of un-concentrated solutions or broth would typically increase incubation time in order to collect the enriched or purified enzyme precipitate.

### B. Uses for Hybrid Glucoamylase Polypeptides

The present invention is also directed to use of a hybrid glucoamylase polypeptide or composition of the present invention in a liquefaction, a saccharification and/or a fermentation process. The hybrid glucoamylase polypeptide or composition may be used in a single process, for example, in a liquefaction process, a saccharification process, or a fermentation process. The hybrid glucoamylase polypeptide or composition may also be used in a combination of processes for example in a liquefaction and saccharification process, in a liquefaction and fermentation process, or in a saccharification and fermentation process, preferably in relation to starch conversion.

### 1. Saccharification

The liquefied starch may be saccharified into a syrup rich in lower DP (e.g., DP1+DP2) saccharides, using alpha-amylases and hybrid glucoamylase polypeptides, optionally in the presence of another enzyme(s). The exact composition of the products of saccharification depends on the combination of enzymes used, as well as the type of starch processed. Advantageously, the syrup obtainable using the provided hybrid glucoamylase polypeptide may contain a weight percent of DP1 of the total oligosaccharides in the saccharified starch exceeding 90%, e.g., 90%-98% or 95%-97%. The weight percent of DP2 in the saccharified starch may be as low as possible, about less than 3%, e.g., 0-3% or 0-2.8%.

Whereas liquefaction is generally run as a continuous process, saccharification is often conducted as a batch process. Saccharification conditions are dependent upon the nature of the liquefact and type of enzymes available. In some cases, a saccharification process may involve temperatures of about 60-65° C. and a pH of about 4.0-4.5, e.g., pH 4.3. Saccharification may be performed, for example, at a temperature between about 40° C., about 50° C., or about 55° C. to about 60° C. or about 65° C., necessitating cooling of the Liquefact. The pH may also be adjusted as needed. Saccharification is normally conducted in stirred tanks, which may take several hours to fill or empty. Enzymes typically are added either at a fixed ratio to dried solids, as the tanks are filled, or added as a single dose at the commencement of the filling stage. A saccharification reaction to make a syrup typically is run over about 24-72 hours, for example, 24-48 hours. A pre-saccharification can be added before saccharification in a simultaneous saccharification and fermentation (SSF), for typically 40-90 minutes at a temperature between 30-65° C., typically about 60° C.

### 2. Raw Starch Hydrolysis

The present invention provides a use of the hybrid glucoamylase polypeptides of the invention for producing glucose and the like from raw starch or granular starch. Generally, the hybrid glucoamylase polypeptide of the present invention either alone or in the presence of an alpha-amylase can be used in raw starch hydrolysis (RSH) or granular starch hydrolysis (GSH) process for producing desired sugars and fermentation products. The granular starch is solubilized by enzymatic hydrolysis below the gelatinization temperature. Such “low-temperature” systems (known also as “no-cook” or “cold-cook”) have been reported to be able to process higher concentrations of dry solids than conventional systems (e.g., up to 45%).

A “raw starch hydrolysis” process (RSH) differs from conventional starch treatment processes, including sequentially or simultaneously saccharifying and fermenting granular starch at or below the gelatinization temperature of the starch substrate typically in the presence of at least a glucoamylase and/or amylase.

The hybrid glucoamylase polypeptide of the invention may also be used in combination with an enzyme that hydrolyzes only alpha-(1,6)-glucosidic bonds in molecules comprising at least four glucosyl residues. Preferably, the hybrid glucoamylase polypeptide of the invention is used in combination with pullulanase or isoamylase. The use of isoamylase and pullulanase for debranching of starch, the molecular properties of the enzymes, and the potential use of the enzymes together with glucoamylase is described in G. M. A. van Beynum et al., Starch Conversion Technology, Marcel Dekker, New York, 1985, 101-142.

### 3. Fermentation

The soluble starch hydrolysate, particularly a glucose rich syrup, can be fermented by contacting the starch hydrolysate with a fermenting organism typically at a temperature around 32° C., such as from 30° C. to 35° C. “Fermenting organism” refers to any organism, including bacterial and fungal organisms, suitable for use in a fermentation process and capable of producing desired a fermentation product. Especially suitable fermenting organisms are able to ferment, i.e., convert, sugars, such as glucose or maltose, directly or indirectly into the desired fermentation product. Examples of fermenting organisms include yeast, such as Saccharomyces cerevisiae and bacteria, e.g., Zymomonas mobilis, expressing alcohol dehydrogenase and pyruvate decarboxylase. The ethanologenic microorganism can express xylose reductase and xylitol dehydrogenase, which convert xylose to xylulosc. Improved strains of ethanologenic microorganisms, which can withstand higher temperatures, for example, are known in the art and can be used. See Liu et al. (2011) Sheng Wu Gong Cheng Xue Bao 27:1049-56. Yeast that can be used for alcohol production include, but are not limited to, Saccharomyces spp., including S. cerevisiae, as well as Kluyveromyces, Lachancea and Schizosaccharomyces spp. Numerous yeast strains are commercially available, many of which have been selected or genetically engineered for desired characteristics, such as high alcohol production, rapid growth rate, and the like. The temperature and pH of the fermentation will depend upon the fermenting organism. Microorganisms that produce other metabolites, such as citric acid and lactic acid, by fermentation are also known in the art. See, e.g., Papagianni (2007) Biotechnol. Adv. 25:244-63; John et al. (2009) Biotechnol. Adv. 27:145-52.

The saccharification and fermentation processes may be carried out as an SSF process. An SSF process can be conducted, in come embodiments, with fungal cells that express and secrete a variant glucoamylase continuously throughout SSF. The fungal cells expressing the hybrid glucoamylase polypeptide also can be the fermenting microorganism, e.g., an ethanologenic microorganism. Ethanol production thus can be carried out using a fungal cell that expresses sufficient hybrid glucoamylase polypeptide so that less or no enzyme has to be added exogenously. The fungal host cell can be selected from an appropriately engineered fungal strains. Fungal host cells that express and secrete other enzymes, in addition to a hybrid glucoamylase polypeptide, also can be used. Such cells may express amylase and/or a pullulanase, phytase, alpha-glucosidase, isoamylase, beta-amylase cellulase, xylanase, other hemicellulases, protease, beta-glucosidase, pectinase, esterase, redox enzymes, transferase, or other enzymes. Fermentation may be followed by subsequent recovery of ethanol.

### 4. Fermentation Products

The term “fermentation product” means a product produced by a process including a fermentation process using a fermenting organism. Fermentation products contemplated according to the invention include alcohols (e.g., arabinitol, butanol, ethanol, glycerol, methanol, ethylene glycol, propylene glycol, butanediol, glycerin, sorbitol, and xylitol); organic acids (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); ketones (e.g., acetone); amino acids (e.g., aspartic acid, glutamic acid, glycine, lysine, serine, and threonine); an alkane (e.g., pentane, hexane, heptane, octane, nonane, decane, undecane, and dodecane); a cycloalkane (e.g., cyclopentane, cyclohexane, cycloheptane, and cyclooctane); an alkene (e.g. pentene, hexene, heptene, and octene); gases (e.g., methane, hydrogen (H2), carbon dioxide (CO2), and carbon monoxide (CO)); antibiotics (e.g., penicillin and tetracycline); enzymes; vitamins (e.g., riboflavin, B12, beta-carotene); and hormones.

In a preferred aspect the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred fermentation processes used include alcohol fermentation processes, which are well known in the art. Preferred fermentation processes are anaerobic fermentation processes, which are well known in the art.

### 5. Brewing

Processes for making beer are well known in the art. See, e.g., Wolfgang Kunze (2004) “Technology Brewing and Malting” Research and Teaching Institute of Brewing, Berlin (VLB), 3rd edition. Briefly, the process involves: (a) preparing a mash, (b) filtering the mash to prepare a wort, and (c) fermenting the wort to obtain a fermented beverage, such as beer.

The brewing composition comprising a hybrid glucoamylase polypeptide, in combination with an amylase and optionally a pullulanase and/or isoamylase, may be added to the mash of step (a) above, i.e., during the preparation of the mash. Alternatively, or in addition, the brewing composition may be added to the mash of step (b) above, i.e., during the filtration of the mash. Alternatively, or in addition, the brewing composition may be added to the wort of step (c) above, i.e., during the fermenting of the wort.

### 6. Animal Nutrition

The hybrid glucoamylase polypeptides and the compositions described herein can be used as a feed additive for animals to increase starch digestibility. Describe herein is a method for increasing starch digestibility in an animal by administering the feed in combination with a hybrid glucoamylase polypeptide disclosed herein.

The term “animal” refers to any organism belonging to the kingdom Animalia and includes, without limitation, mammals (excluding humans), non-human animals, domestic animals, livestock, farm animals, zoo animals, breeding stock and the like. For example, there can be mentioned all non-ruminant and ruminant animals. In an embodiment, the animal is a non-ruminant, i.e., a mono-gastric animal. Examples of mono-gastric animals include, but are not limited to, pigs and swine, such as piglets, growing pigs, sows; poultry such as turkeys, ducks, chicken, broiler chicks, layers; fish such as salmon, trout, tilapia, catfish and carps; and crustaceans such as shrimps and prawns. In a further embodiment, the animal is a ruminant animal including, but not limited to, cattle, young calves, goats, sheep, giraffes, bison, moose, elk, yaks, water buffalo, deer, camels, alpacas, llamas, antelope, pronghorn and nilgai.

The terms “animal feed”, “feed”, “feedstuff” and “fodder” are used interchangeably and can comprise one or more feed materials selected from the group comprising a) cereals, such as small grains (e.g., wheat, barley, rye, oats and combinations thereof) and/or large grains such as maize or sorghum; b) byproducts from cereals, such as corn gluten meal, Distillers Dried Grains with Solubles (DDGS) (particularly corn based Distillers Dried Grains with Solubles (cDDGS), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; and/or c) minerals and vitamins.

The digestibility of starch in feeds is highly variable and dependent on a number of factors including the physical structure of both the starch and feed matrix. It has been found that starch digestibility in an animal's diet can be improved by the use of at least one glucoamylase as a feed additive.

When used as, or in the preparation of, a feed, such as functional feed, the enzyme or feed additive composition of the present invention may be used in conjunction with one or more of: a nutritionally acceptable carrier, a nutritionally acceptable diluent, a nutritionally acceptable excipient, a nutritionally acceptable adjuvant, a nutritionally active ingredient. For example, there could be mentioned at least one component selected from the group consisting of a protein, a peptide, sucrose, lactose, sorbitol, glycerol, propylene glycol, sodium chloride, sodium sulfate, sodium acetate, sodium citrate, sodium formate, sodium sorbate, potassium chloride, potassium sulfate, potassium acetate, potassium citrate, potassium formate, potassium acetate, potassium sorbate, magnesium chloride, magnesium sulfate, magnesium acetate, magnesium citrate, magnesium formate, magnesium sorbate, sodium metabisulfite, methyl paraben and propyl paraben.

It is also possible that at least one hybrid glucoamylase polypeptide (or an enzyme composition comprising at least one hybrid glucoamylase polypeptide) described herein can be homogenized to produce a powder. The powder may be mixed with other components known in the art. Optionally, the feedstuff may also contain additional minerals such as, for example, calcium and/or additional vitamins. In some embodiments, the feedstuff is a corn soybean meal mix.

In an alternative preferred embodiment, an enzyme composition comprising at least one hybrid glucoamylase polypeptide can be formulated to granules as described in WO2007/044968 (referred to as TPT granules) or WO1997/016076 or WO1992/012645 incorporated herein by reference. “TPT” means Thermo Protection Technology. When the feed additive composition is formulated into granules, the granules comprise a hydrated barrier salt coated over the protein core. The advantage of such salt coating is improved thermotolerance, improved storage stability and protection against other feed additives otherwise having adverse effect on the enzyme. Preferably, the salt used for the salt coating has a water activity greater than 0.25 or constant humidity greater than 60% at 20° C. In some embodiments, the salt coating comprises Na₂SO₄.

Alternatively, the composition is in a liquid formulation suitable for consumption preferably such liquid consumption contains one or more of the following: a buffer, salt, sorbitol and/or glycerol.

Any of the hybrid glucoamylase polypeptides described herein for use as a feed additive may be used alone or in combination with at least one direct fed microbial. Categories of DFMs include Bacillus, Lactic Acid Bacteria and Yeasts. Further, any of the glucoamylases described herein for use as a feed additive may be used alone or in combination with at least one essential oil, for example cinnamaldehyde and/or thymol. Still further, any of the glucoamylases described herein for use as a feed additive may be used alone or in combination with at least one additional enzyme. Examples of such enzymes include, without limitation, phytases, xylanases, proteases, amylases, glucanases, or other glucoamylases.

Also disclosed is a method for improving the nutritional value of an animal feed, wherein an effective amount of any of the hybrid glucoamylase polypeptides described herein can be added to animal feed.

The phrase, an “effective amount” as used herein, refers to the amount of an active agent (such as any of the hybrid glucoamylase polypeptides disclosed herein) required to confer improved performance on an animal on one or more metrics, either alone or in combination with one or more other active agents (such as, without limitation, one or more additional enzyme(s), one or more DFM(s), one or more essential oils, etc.).

The term “animal performance” as used herein may be determined by any metric such as, without limitation, the feed efficiency and/or weight gain of the animal and/or by the feed conversion ratio and/or by the digestibility of a nutrient in a feed and/or digestible energy or metabolizable energy in a feed and/or by animals' ability to avoid the negative effects of diseases or by the immune response of the subject.

Animal performance characteristics may include but are not limited to: body weight; weight gain; mass; body fat percentage; height; body fat distribution; growth; growth rate; egg size; egg weight; egg mass; egg laying rate; mineral absorption; mineral excretion, mineral retention; bone density; bone strength; feed conversion rate (FCR); average daily feed intake (ADFI); Average daily gain (ADG) retention and/or a secretion of any one or more of copper, sodium, phosphorous, nitrogen and calcium; amino acid retention or absorption; mineralization, bone mineralization carcass yield and carcass quality.

By “improved animal performance on one or more metric” it is meant that there is increased feed efficiency, and/or increased weight gain and/or reduced feed conversion ratio and/or improved digestibility of nutrients or energy in a feed and/or by improved nitrogen retention and/or by improved ability to avoid the negative effects of necrotic enteritis and/or by an improved immune response in the subject resulting from the use of feed comprising the feed additive composition described herein as compared to a feed which does not comprise said feed additive composition.

All references cited herein are herein incorporated by reference in their entirety for all purposes. In order to further illustrate the compositions and methods, and advantages thereof, the following specific examples are given with the understanding that they are illustrative rather than limiting.

### EXAMPLES

### Example 1

Sequences of the hybrid variants of Saksenaea vasiformis B4078 glucoamylase The polypeptides included in the invention were produced by fusing a selection of polypeptides containing carbohydrate binding module (CBM) at N-terminus and/or C-termini of the catalytic domain of SvaGA1 (SEQ ID NO: 41) or a variant SvaGA1v2 (SEQ ID NO: 3). In constructs where the CBM+linker was added at the N-terminus, the signal peptide of SvaGA1 and SvaGA1v2 (predicted by SignalP software version 4.0 (Nordahl Petersen et al. (2011) Nature Methods, 8:785-786)) was replaced by the signal peptide natively linked with the CBM sequence used.

The protein and DNA sequences for various catalytic domains (CD), prelinkers (sequence between CD and linkers), linkers, and carbohydrate binding modules (CBM) evaluated in the subsequent studies are shown on Table 1. The abbreviation “RSA” refers to “Mycological Collections of the Rancho Santa Ana Botanic Garden”.

TABLE 1
Sequences numbers of catalytic domains (CD), prelinkers, linkers, and carbohydrate
binding modules (CBM) sequences used in subsequent studies. AA and GA are
the abbreviations for alpha-amylase and glucoamylase, respectively.

| | | | Sequence |
|---|---|---|---|
| Type | Sequence origin | SEQ ID | type |
| Signal | Mucor cordense RSA 1222 GA | SEQ ID NO: 1 | PROTEIN |
| peptide + CBM + linker | | | |
| Signal | Mucor cordense RSA 1222 GA | SEQ ID NO: 2 | DNA |
| peptide + CBM + linker | | | |
| Catalytic domain | Saksenaea vasiformis B4078 | SEQ ID NO: 3 | PROTEIN |
| | GA variant | | |
| Catalytic domain | Saksenaea vasiformis B4078 | SEQ ID NO: 4 | DNA |
| | GA variant | | |
| Signal | Backusella circina FSU 941 GA | SEQ ID NO: 5 | PROTEIN |
| peptide + CBM + linker | | | |
| Signal | Backusella circina FSU 941 GA | SEQ ID NO: 6 | DNA |
| peptide + CBM + linker | | | |
| Signal | Dichotomocladium elegans | SEQ ID NO: 7 | PROTEIN |
| peptide + CBM + linker | RSA 919- GA | | |
| Signal | Dichotomocladium elegans | SEQ ID NO: 8 | DNA |
| peptide + CBM + linker | RSA 919- GA | | |
| Signal | Fennellomyces sp. T-0311 GA | SEQ ID NO: 9 | PROTEIN |
| peptide + CBM + linker | | | |
| Signal | Fennellomyces sp. T-0311 GA | SEQ ID NO: 10 | DNA |
| peptide + CBM + linker | | | |
| Signal | Phascolomyces articulosus GA | SEQ ID NO: 11 | PROTEIN |
| peptide + CBM + linker | | | |
| Signal | Phascolomyces articulosus GA | SEQ ID NO: 12 | DNA |
| peptide + CBM + linker | | | |
| Signal | Syncephalastrum racemosum | SEQ ID NO: 13 | PROTEIN |
| peptide + CBM + linker | NRRL 2496 GA | | |
| Signal | Syncephalastrum racemosum | SEQ ID NO: 14 | DNA |
| peptide + CBM + linker | NRRL 2496 GA | | |
| Signal | Choanephora cucurbitarum | SEQ ID NO: 15 | PROTEIN |
| peptide + CBM + linker | NRRL2744 (50) GA | | |
| Signal | Choanephora cucurbitarum | SEQ ID NO: 16 | DNA |
| peptide + CBM + linker | NRRL2744 (50) GA | | |
| Signal | Rhizopus stolonifer GA | SEQ ID NO: 17 | PROTEIN |
| peptide + CBM + linker | | | |
| Signal | Rhizopus stolonifer GA | SEQ ID NO: 18 | DNA |
| peptide + CBM + linker | | | |
| Signal | Mucor circinelloides f. | SEQ ID NO: 19 | PROTEIN |
| peptide + CBM + linker | circinelloides 1006PhL GA | | |
| Signal | Mucor circinelloides f. | SEQ ID NO: 20 | DNA |
| peptide + CBM + linker | circinelloides 1006PhL GA | | |
| Signal | Dactylellina haptotyla CBS | SEQ ID NO: 21 | PROTEIN |
| peptide + CBM + linker | 200.50 AA | | |
| Signal | Dactylellina haptotyla CBS | SEQ ID NO: 22 | DNA |
| peptide + CBM + linker | 200.50 AA | | |
| Signal | Lipomyces starkeyi AA | SEQ ID NO: 23 | PROTEIN |
| peptide + CBM + linker | | | |
| Signal | Lipomyces starkeyi AA | SEQ ID NO: 24 | DNA |
| peptide + CBM + linker | | | |
| Signal | Rhizopus microsporus var. | SEQ ID NO: 25 | PROTEIN |
| peptide + CBM + linker | microsporus ATCC52813 GA | | |
| Signal | Rhizopus microsporus var. | SEQ ID NO: 26 | DNA |
| peptide + CBM + linker | microsporus ATCC52813 GA | | |
| Prelinker | Aspergillus niger GA | SEQ ID NO: 27 | PROTEIN |
| Prelinker | Aspergillus niger GA | SEQ ID NO: 28 | DNA |
| Linker | Aspergillus niger GA | SEQ ID NO: 29 | PROTEIN |
| Linker | Aspergillus niger GA | SEQ ID NO: 30 | DNA |
| CBM | Aspergillus niger GA | SEQ ID NO: 31 | PROTEIN |
| CBM | Aspergillus niger GA | SEQ ID NO: 32 | DNA |
| Signal | Saksenaea vasiformis B4078 | SEQ ID NO: 33 | PROTEIN |
| peptide + Catalytic | GA variant | | |
| Domain | | | |
| Signal | Saksenaea vasiformis B4078 | SEQ ID NO: 34 | DNA |
| peptide + Catalytic | GA variant | | |
| Domain | | | |
| Prelinker | Trichoderma reesei GA | SEQ ID NO: 35 | PROTEIN |
| Prelinker | Trichoderma reesei GA | SEQ ID NO: 36 | DNA |
| Linker | Trichoderma reesei GA | SEQ ID NO: 37 | PROTEIN |
| Linker | Trichoderma reesei GA | SEQ ID NO: 38 | DNA |
| CBM | Trichoderma reesei GA | SEQ ID NO: 39 | PROTEIN |
| CBM | Trichoderma reesei GA | SEQ ID NO: 40 | DNA |
| Signal | Saksenaea vasiformis B4078 | SEQ ID NO: 41 | PROTEIN |
| peptide + Catalytic | GA | | |
| Domain | | | |
| Signal | Saksenaea vasiformis B4078 | SEQ ID NO: 42 | DNA |
| peptide + Catalytic | GA | | |
| Domain | | | |
| Linker | Aspergillus niger GA | SEQ ID NO: 43 | DNA |
| CBM | Aspergillus niger GA | SEQ ID NO: 44 | DNA |

TABLE 2
The architecture of hybrid SvaGA1 variants

| | N-terminal | Catalytic | | | C-terminal |
|---|---|---|---|---|---|
| Designation | CBM | domain | Prelinker | Linker | CBM |
| SvaGA1v125 | SEQ ID NO: | SEQ ID NO: | — | — | — |
| | 1 | 3 | | | |
| SvaGA1v126 | SEQ ID NO: | SEQ ID NO: | — | — | — |
| | 5 | 3 | | | |
| SvaGA1v127 | SEQ ID NO: | SEQ ID NO: | — | — | — |
| | 7 | 3 | | | |
| SvaGA1v128 | SEQ ID NO: | SEQ ID NO: | — | — | — |
| | 9 | 3 | | | |
| SvaGA1v129 | SEQ ID NO: | SEQ ID NO: | — | — | — |
| | 11 | 3 | | | |
| SvaGA1v130 | SEQ ID NO: | SEQ ID NO: | — | — | — |
| | 13 | 3 | | | |
| SvaGA1v131 | SEQ ID NO: | SEQ ID NO: | — | — | — |
| | 15 | 3 | | | |
| SvaGA1v132 | SEQ ID NO: | SEQ ID NO: | — | — | — |
| | 17 | 3 | | | |
| SvaGA1v133 | SEQ ID NO: | SEQ ID NO: | — | — | — |
| | 19 | 3 | | | |
| SvaGA1v134 | SEQ ID NO: | SEQ ID NO: | — | — | — |
| | 21 | 3 | | | |
| SvaGA1v135 | SEQ ID NO: | SEQ ID NO: | — | — | — |
| | 23 | 3 | | | |
| SvaGA1v052 | SEQ ID NO: | SEQ ID NO: | — | — | — |
| | 25 | 3 | | | |
| SvaGA1v435 | SEQ ID NO: | SEQ ID NO: | SEQ ID | SEQ ID | SEQ ID |
| | 19 | 3 | NO: 27 | NO: 29 | NO: 31 |
| SvaGA1v436 | SEQ ID NO: | SEQ ID NO: | SEQ ID | SEQ ID | SEQ ID |
| | 1 | 3 | NO: 27 | NO: 29 | NO: 31 |
| SvaGA1v437 | — | SEQ ID NO: | SEQ ID | SEQ ID | SEQ ID |
| | | 33 | NO: 27 | NO: 29 | NO: 31 |
| SvaGA1v440 | — | SEQ ID NO: | SEQ ID | SEQ ID | SEQ ID |
| | | 33 | NO: 35 | NO: 37 | NO: 39 |
| SvaGA1v4 | — | SEQ ID NO: | SEQ ID | SEQ ID | SEQ ID |
| | | 41 | NO: 27 | NO: 29 | NO: 31 |
| SvaGA1v6 | — | SEQ ID NO: | — | SEQ ID | SEQ ID |
| | | 41 | | NO: 29 | NO: 31 |

The architecture of glucoamylase protein variants based on the SvaGA1 catalytic domain constructed in the invention are shown in Table 2. Numerous CBMs were incorporated at either the N or C-terminus of the glucoamylase catalytic domains.

The sequence IDs of the hybrid variants are listed in Table 3 below.

TABLE 3
Sequence IDs of hybrid variants

| Seq Name | SEQ ID | Sequence type |
|---|---|---|
| SvaGA1v52 | SEQ ID NO: 75 | Protein |
| SvaGA1v125 | SEQ ID NO: 76 | Protein |
| SvaGA1v126 | SEQ ID NO: 77 | Protein |
| SvaGA1v127 | SEQ ID NO: 78 | Protein |
| SvaGA1v128 | SEQ ID NO: 79 | Protein |
| SvaGA1v129 | SEQ ID NO: 80 | Protein |
| SvaGA1v130 | SEQ ID NO: 81 | Protein |
| SvaGA1v131 | SEQ ID NO: 82 | Protein |
| SvaGA1v132 | SEQ ID NO: 83 | Protein |
| SvaGA1v133 | SEQ ID NO: 84 | Protein |
| SvaGA1v134 | SEQ ID NO: 85 | Protein |
| SvaGA1v435 | SEQ ID NO: 86 | Protein |
| SvaGA1v436 | SEQ ID NO: 87 | Protein |
| SvaGA1v437 | SEQ ID NO: 88 | Protein |
| SvaGA1v440 | SEQ ID NO: 89 | Protein |
| SvaGA1v52 | SEQ ID NO: 90 | DNA |
| SvaGA1v125 | SEQ ID NO: 91 | DNA |
| SvaGA1v126 | SEQ ID NO: 92 | DNA |
| SvaGA1v127 | SEQ ID NO: 93 | DNA |
| SvaGA1v128 | SEQ ID NO: 94 | DNA |
| SvaGA1v129 | SEQ ID NO: 95 | DNA |
| SvaGA1v130 | SEQ ID NO: 96 | DNA |
| SvaGA1v131 | SEQ ID NO: 97 | DNA |
| SvaGA1v132 | SEQ ID NO: 98 | DNA |
| SvaGA1v133 | SEQ ID NO: 99 | DNA |
| SvaGA1v134 | SEQ ID NO: 100 | DNA |
| SvaGA1v435 | SEQ ID NO: 101 | DNA |
| SvaGA1v436 | SEQ ID NO: 102 | DNA |
| SvaGA1v437 | SEQ ID NO: 103 | DNA |
| SvaGA1v440 | SEQ ID NO: 104 | DNA |

### Example 2

Expression of Hybrid GA Variants in Trichoderma reesei

The polynucleotides (codon modified sequences used as expression cassettes) encoding the GA variant sequences were synthesized by Generay (Generay Biotech Co., Ltd, Shanghai, China) and inserted into the pGX256 expression vector, a derivative vector from pTTT (see, Published U.S. patent application No. 20110020899). All plasmids were transformed into a suitable Trichoderma reesei strain using protoplast transformation (Te'o et al., J. Microbiol. Methods 51:393-99, 2002). The transformants were selected and fermented by the methods described in WO 2016/138315. Supernatants from these cultures were used to confirm the Protein expression by SDS-PAGE analysis.

Fungal cell cultures were grown in a defined medium as described by Lv et al (2012) in “Construction of two vectors for gene expression in Trichoderma reesei”. Plasmids 67:67-71. Clarified culture broth were collected after 96 hours by centrifugation. The glucoamylases variants were purified by methods known in the art. The column chromatography fractions containing the target Protein were pooled, concentrated and equilibrated to 20 mM sodium acetate pH 5.0, 150 mM sodium chloride using an Amicon Ultra-15 device with 10 K MWCO. The purified samples were approximately 99% pure (by SDS-PAGE analysis) and were stored in 40% glycerol at −80° C. until use.

### Example 3

### Corn Starch Hydrolyzing Activity of Hybrid SvaGA1v2 Variants

The substrate used in this assay was 1% (w/v) corn starch (Sigma, Cat. No. S4126) in 50 mM sodium acetate buffer (pH 4.5). The hybrid glucoamylase variants were tested for their corn starch hydrolyzing activity in combination with the fungal alpha-amylase from Aspergillus terreus. The reaction was initiated by adding 10 μL of glucoamylase and 10 μL of alpha-amylase to 90 μL of the substrate, with final dosages at 10 ppm and 1.5 ppm for glucoamylase and alpha-amylase, respectively. The incubations were done in iEMS (32° C., 900 rpm) for 60 min. After that, the glucose release was measured using the coupled glucose oxidase/peroxidase (GOX/HRP) and 2,2′-Azino-bis 3-ethylbenzothiazoline-6-sulfonic acid (ABTS) method (Anal. Biochem. 105 (1980), 389-397). The reaction mixture (10 uL) was transferred into 90 μL of ABTS/GOX/HRP solution (2.74 mg/mL ABTS, 0.1 U/mL HRP, and 1 U/mL GOX). Absorbance at 405 nm was immediately measured at 11 seconds intervals for 5 min using a SoftMax Pro plate reader (Molecular Device). The output was the reaction rate, Vo, and it was used to indicate the corn starch hydrolyzing activity of the hybrid glucoamylase variants. As shown in Table 4, all the hybrid SvaGA1v2 variants showed higher corn starch hydrolyzing activity than SvaGA1v2. Moreover, all the hybrid variants also outperformed a commercially relevant Trichoderma reesei variant glucoamylase (previously described in WO2009/067218) under the conditions tested.

TABLE 4
Corn starch hydrolyzing activity of hybrid SvaGA1
variants measured as rate of glucose release.

| | Sample | Reaction Rate |
|---|---|---|
| Gene structure | name | (Vₒ) |
| C-CBM20 | SvaGA1v437 | 92.1 |
| N-CBM21 | SvaGA1v440 | 77.6 |
| | SvaGA1v125 | 101.8 |
| | SvaGA1v126 | 107.9 |
| | SvaGA1v127 | 110.9 |
| | SvaGA1v128 | 104.7 |
| | SvaGA1v129 | 85.8 |
| | SvaGA1v130 | 91.2 |
| | SvaGA1v131 | 81.7 |
| | SvaGA1v132 | 89.9 |
| | SvaGA1v133 | 100.6 |
| | SvaGA1v134 | 94.7 |
| | SvaGA1v52 | 86.0 |
| N-CBM21-C- | SvaGA1v435 | 86.6 |
| CBM20 | SvaGA1v436 | 81.5 |
| Benchmark | SvaGA1v2 | 41.3 |
| | TrGA variant | 66.5 |

### Example 4

### Sequence of Hybrid Variants of Additional Mucorales-Clade Glucoamylases

The polypeptides included in the invention were produced by fusing a selection of polypeptides containing starch binding domains (CBM) at N-terminus and/or C-terminus of the catalytic domain of Mucorales-clade glucoamylases or their variants. In constructs where the CBM+linker was added at the N-terminus, the signal peptide of those glucoamylases (predicted by SignalP software version 4.0 (Nordahl Petersen et al. (2011) Nature Methods, 8:785-786)) was replaced by the signal peptide natively linked with the CBM sequence used.

The amino acid and DNA sequences numbers of various catalytic domains (CD), prelinkers (sequence between CD and linkers), linkers, carbohydrate binding modules (CBM) used in subsequent studies are listed in Table 5.

TABLE 5
Sequences numbers of catalytic domains (CD), prelinkers, linkers, carbohydrate
binding modules (CBM). GA is the abbreviation for glucoamylase.

| | | | Sequence |
|---|---|---|---|
| Type | Sequence origin | SEQ ID | type |
| Signal peptide | Saksenaea oblongispora CDC-B3353 | SEQ ID NO: | Protein |
| | GA | 45 | |
| Signal peptide | Saksenaea oblongispora CDC-B3353 | SEQ ID NO: | DNA |
| | GA | 46 | |
| Catalytic Domain | Saksenaea oblongispora CDC-B3353 | SEQ ID NO: | Protein |
| | GA | 47 | |
| Catalytic Domain | Saksenaea oblongispora CDC-B3353 | SEQ ID NO: | DNA |
| | GA | 48 | |
| Catalytic Domain | Saksenaea oblongispora CDC-B3353 | SEQ ID NO: | Protein |
| | GA variant | 49 | |
| Catalytic Domain | Saksenaea oblongispora CDC-B3353 | SEQ ID NO: | DNA |
| | GA variant | 50 | |
| Signal peptide | Apophysomyces ossiformis GA | SEQ ID NO: | Protein |
| | | 51 | |
| Signal peptide | Apophysomyces ossiformis GA | SEQ ID NO: | DNA |
| | | 52 | |
| Catalytic Domain | Apophysomyces ossiformis GA | SEQ ID NO: | Protein |
| | | 53 | |
| Catalytic Domain | Apophysomyces ossiformis GA | SEQ ID NO: | DNA |
| | | 54 | |
| Catalytic Domain | Apophysomyces ossiformis GA variant | SEQ ID NO: | Protein |
| | | 55 | |
| Catalytic Domain | Apophysomyces ossiformis GA variant | SEQ ID NO: | DNA |
| | | 56 | |
| Signal peptide | Apophysomyces elegans B7760 GA | SEQ ID NO: | Protein |
| | | 57 | |
| Signal peptide | Apophysomyces elegans B7760 GA | SEQ ID NO: | DNA |
| | | 58 | |
| Catalytic Domain | Apophysomyces elegans B7760 GA | SEQ ID NO: | Protein |
| | | 59 | |
| Catalytic Domain | Apophysomyces elegans B7760 GA | SEQ ID NO: | DNA |
| | | 60 | |
| Catalytic Domain | Apophysomyces elegans B7760 GA | SEQ ID NO: | Protein |
| | variant | 61 | |
| Catalytic Domain | Apophysomyces elegans B7760 GA | SEQ ID NO: | DNA |
| | variant | 62 | |
| Signal peptide | Apophysomyces variabilis NCCPF | SEQ ID NO: | Protein |
| | 102052 GA | 63 | |
| Signal peptide | Apophysomyces variabilis NCCPF | SEQ ID NO: | DNA |
| | 102052 GA | 64 | |
| Catalytic Domain | Apophysomyces variabilis NCCPF | SEQ ID NO: | Protein |
| | 102052 GA | 65 | |
| Catalytic Domain | Apophysomyces variabilis NCCPF | SEQ ID NO: | DNA |
| | 102052 GA | 66 | |
| Catalytic Domain | Apophysomyces variabilis NCCPF | SEQ ID NO: | Protein |
| | 102052 GA variant | 67 | |
| Catalytic Domain | Apophysomyces variabilis NCCPF | SEQ ID NO: | DNA |
| | 102052 GA variant | 68 | |
| Signal peptide | Apophysomyces trapeziformis B9324 GA | SEQ ID NO: | Protein |
| | | 69 | |
| Signal peptide | Apophysomyces trapeziformis B9324 GA | SEQ ID NO: | DNA |
| | | 70 | |
| Catalytic Domain | Apophysomyces trapeziformis B9324 GA | SEQ ID NO: | Protein |
| | | 71 | |
| Catalytic Domain | Apophysomyces trapeziformis B9324 GA | SEQ ID NO: | DNA |
| | | 72 | |
| Catalytic Domain | Apophysomyces trapeziformis B9324 GA | SEQ ID NO: | Protein |
| | variant | 73 | |
| Catalytic Domain | Apophysomyces trapeziformis B9324 GA | SEQ ID NO: | DNA |
| | variant | 74 | |

The architecture of various hybrid glucoamylase variants constructed and evaluated in the invention is shown in Table 6. Numerous CBMs were incorporated at either the N or C-terminus of the glucoamylase catalytic domains.

TABLE 6
The architecture of hybrid variants of homologous
Mucorales-clade glucoamylases

| | | N- | | | | C- |
|---|---|---|---|---|---|---|
| | Signal | terminal | Catalytic | | | terminal |
| Designation | peptide | CBM | domain | Prelinker | Linker | CBM |
| CRC24662-WT | SEQ ID | — | SEQ ID | — | — | — |
| | NO: 45 | | NO: 47 | | | |
| CRC24662v2 | SEQ ID | — | SEQ ID | — | — | — |
| | NO: 45 | | NO: 49 | | | |
| CRC24662v2_1 | — | SEQ ID | SEQ ID | — | — | — |
| 9687_CBM21 | | NO: 19 | NO: 49 | | | |
| CRC24662v2_1 | — | SEQ ID | SEQ ID | | | |
| 3978_CBM21 | | NO: 1 | NO: 49 | | | |
| CRC24662v2_A | SEQ ID | — | SEQ ID | SEQ ID | SEQ ID | SEQ ID |
| nGA_CBM20 | NO: 45 | | NO: 49 | NO: 27 | NO: 29 | NO: 31 |
| CRC24662v2_T | SEQ ID | — | SEQ ID | SEQ ID | SEQ ID | SEQ ID |
| rGA_CBM20 | NO: 45 | | NO: 49 | NO: 35 | NO: 37 | NO: 39 |
| CRC24663-WT | SEQ ID | — | SEQ ID | — | — | — |
| | NO: 51 | | NO: 53 | | | |
| CRC24663v2 | SEQ ID | — | SEQ ID | — | — | — |
| | NO: 51 | | NO: 55 | | | |
| CRC24663v2_1 | — | SEQ ID | SEQ ID | — | — | — |
| 9687_CBM21 | | NO: 19 | NO: 55 | | | |
| CRC24663v2_1 | — | SEQ ID | SEQ ID | — | — | — |
| 3978_CBM21 | | NO: 1 | NO: 55 | | | |
| CRC24663v2_A | SEQ ID | — | SEQ ID | SEQ ID | SEQ ID | SEQ ID |
| nGA_CBM20 | NO: 51 | | NO: 55 | NO: 27 | NO: 29 | NO: 31 |
| CRC24663v2_T | SEQ ID | - | SEQ ID | SEQ ID | SEQ ID | SEQ ID |
| rGA_CBM20 | NO: 51 | | NO: 55 | NO: 35 | NO: 37 | NO: 39 |
| CRC24664-WT | SEQ ID | — | SEQ ID | — | — | — |
| | NO: 57 | | NO: 59 | | | |
| CRC24664v2 | SEQ ID | — | SEQ ID | — | — | — |
| | NO: 57 | | NO: 61 | | | |
| CRC24664v2_1 | — | SEQ ID | SEQ ID | — | — | — |
| 9687_CBM21 | | NO: 19 | NO: 61 | | | |
| CRC24664v2_1 | — | SEQ ID | SEQ ID | — | — | — |
| 3978_CBM21 | | NO: 1 | NO: 61 | | | |
| CRC24664v2_A | SEQ ID | — | SEQ ID | SEQ ID | SEQ ID | SEQ ID |
| nGA_CBM20 | NO: 57 | | NO: 61 | NO: 27 | NO: 29 | NO: 31 |
| CRC24664v2_T | SEQ ID | — | SEQ ID | SEQ ID | SEQ ID | SEQ ID |
| rGA_CBM20 | NO: 57 | | NO: 61 | NO: 35 | NO: 37 | NO: 39 |
| CRC24665-WT | SEQ ID | — | SEQ ID | — | — | — |
| | NO: 63 | | NO: 65 | | | |
| CRC24665v2 | SEQ ID | — | SEQ ID | — | — | — |
| | NO: 63 | | NO: 67 | | | |
| CRC24665v2_1 | — | SEQ ID | SEQ ID | — | — | — |
| 9687_CBM21 | | NO: 19 | NO: 67 | | | |
| CRC24665v2_1 | — | SEQ ID | SEQ ID | — | — | — |
| 3978_CBM21 | | NO: 1 | NO: 67 | | | |
| CRC24665v2_A | SEQ ID | — | SEQ ID | SEQ ID | SEQ ID | SEQ ID |
| nGA_CBM20 | NO: 63 | | NO: 67 | NO: 27 | NO: 29 | NO: 31 |
| CRC24665v2_T | SEQ ID | — | SEQ ID | SEQ ID | SEQ ID | SEQ ID |
| rGA_CBM20 | NO: 63 | | NO: 67 | NO: 35 | NO: 37 | NO: 39 |
| CRC24666-WT | SEQ ID | — | SEQ ID | — | — | — |
| | NO: 69 | | NO: 71 | | | |
| CRC24666v2 | SEQ ID | — | SEQ ID | — | — | — |
| | NO: 69 | | NO: 73 | | | |
| CRC24666v2_1 | — | SEQ ID | SEQ ID | — | — | — |
| 9687_CBM21 | | NO: 19 | NO: 73 | | | |
| CRC24666v2_1 | — | SEQ ID | SEQ ID | — | — | — |
| 3978_CBM21 | | NO: 1 | NO: 73 | | | |
| CRC24666v2_A | SEQ ID | — | SEQ ID | SEQ ID | SEQ ID | SEQ ID |
| nGA_CBM20 | NO: 69 | | NO: 73 | NO: 27 | NO: 29 | NO: 31 |
| CRC24666v2_T | SEQ ID | — | SEQ ID | SEQ ID | SEQ ID | SEQ ID |
| rGA_CBM20 | NO: 69 | | NO: 73 | NO: 35 | NO: 37 | NO: 39 |

The sequence IDs of those resulted hybrid variants were listed in Table 7.

TABLE 7
Sequence IDs of additional Mucorales-clade glucoamylase variants

| Seq Name | SEQ ID | Sequence type |
|---|---|---|
| CRC24662v2 | SEQ ID NO: 105 | Protein |
| CRC24662v2_19687_CBM21 | SEQ ID NO: 106 | Protein |
| CRC24662v2_13978_CBM21 | SEQ ID NO: 107 | Protein |
| CRC24662v2_AnGA_CBM20 | SEQ ID NO: 108 | Protein |
| CRC24662v2_TrGA_CBM20 | SEQ ID NO: 109 | Protein |
| CRC24663v2 | SEQ ID NO: 110 | Protein |
| CRC24663v2_19687_CBM21 | SEQ ID NO: 111 | Protein |
| CRC24663v2_13978_CBM21 | SEQ ID NO: 112 | Protein |
| CRC24663v2_AnGA_CBM20 | SEQ ID NO: 113 | Protein |
| CRC24663v2_TrGA_CBM20 | SEQ ID NO: 114 | Protein |
| CRC24664v2 | SEQ ID NO: 115 | Protein |
| CRC24664v2_19687_CBM21 | SEQ ID NO: 116 | Protein |
| CRC24664v2_13978_CBM21 | SEQ ID NO: 117 | Protein |
| CRC24664v2_AnGA_CBM20 | SEQ ID NO: 118 | Protein |
| CRC24664v2_TrGA_CBM20 | SEQ ID NO: 119 | Protein |
| CRC24665v2 | SEQ ID NO: 120 | Protein |
| CRC24665v2_19687_CBM21 | SEQ ID NO: 121 | Protein |
| CRC24665v2_13978_CBM21 | SEQ ID NO: 122 | Protein |
| CRC24665v2_AnGA_CBM20 | SEQ ID NO: 123 | Protein |
| CRC24665v2_TrGA_CBM20 | SEQ ID NO: 124 | Protein |
| CRC24666v2 | SEQ ID NO: 125 | Protein |
| CRC24666v2_19687_CBM21 | SEQ ID NO: 126 | Protein |
| CRC24666v2_13978_CBM21 | SEQ ID NO: 127 | Protein |
| CRC24666v2_AnGA_CBM20 | SEQ ID NO: 128 | Protein |
| CRC24666v2_TrGA_CBM20 | SEQ ID NO: 129 | Protein |
| CRC24662v2 | SEQ ID NO: 130 | DNA |
| CRC24662v2_19687_CBM21 | SEQ ID NO: 131 | DNA |
| CRC24662v2_13978_CBM21 | SEQ ID NO: 132 | DNA |
| CRC24662v2_AnGA_CBM20 | SEQ ID NO: 133 | DNA |
| CRC24662v2_TrGA_CBM20 | SEQ ID NO: 134 | DNA |
| CRC24663v2 | SEQ ID NO: 135 | DNA |
| CRC24663v2_19687_CBM21 | SEQ ID NO: 136 | DNA |
| CRC24663v2_13978_CBM21 | SEQ ID NO: 137 | DNA |
| CRC24663v2_AnGA_CBM20 | SEQ ID NO: 138 | DNA |
| CRC24663v2_TrGA_CBM20 | SEQ ID NO: 139 | DNA |
| CRC24664v2 | SEQ ID NO: 140 | DNA |
| CRC24664v2_19687_CBM21 | SEQ ID NO: 141 | DNA |
| CRC24664v2_13978_CBM21 | SEQ ID NO: 142 | DNA |
| CRC24664v2_AnGA_CBM20 | SEQ ID NO: 143 | DNA |
| CRC24664v2_TrGA_CBM20 | SEQ ID NO: 144 | DNA |
| CRC24665v2 | SEQ ID NO: 145 | DNA |
| CRC24665v2_19687_CBM21 | SEQ ID NO: 146 | DNA |
| CRC24665v2_13978_CBM21 | SEQ ID NO: 147 | DNA |
| CRC24665v2_AnGA_CBM20 | SEQ ID NO: 148 | DNA |
| CRC24665v2_TrGA_CBM20 | SEQ ID NO: 149 | DNA |
| CRC24666v2 | SEQ ID NO: 150 | DNA |
| CRC24666v2_19687_CBM21 | SEQ ID NO: 151 | DNA |
| CRC24666v2_13978_CBM21 | SEQ ID NO: 152 | DNA |
| CRC24666v2_AnGA_CBM20 | SEQ ID NO: 153 | DNA |
| CRC24666v2_TrGA_CBM20 | SEQ ID NO: 154 | DNA |

### Example 5

### Corn Starch Hydrolyzing Activity of Hybrid Variants of Additional Mucorales-Clade Glucoamylases

The substrate used in this assay was 1% (w/v) corn starch (Sigma, Cat. No. S4126) in 50 mM sodium acetate buffer (pH 4.5). The hybrid glucoamylase variants were tested for their corn starch hydrolyzing activity in the presence of the fungal alpha-amylase from Aspergillus terreus. The reaction was initiated by adding 10 μL of glucoamylase and 10 μL of alpha-amylase to 90 μL of the substrate, with final dosages at 10 ppm and 1.5 ppm for glucoamylase and alpha-amylase, respectively. The incubations were carried out in iEMS (32° C., 900 rpm) for 60 min. The glucose release was measured using the coupled glucose oxidase/peroxidase (GOX/HRP) and 2,2′-Azino-bis 3-ethylbenzothiazoline-6-sulfonic acid (ABTS) method (Anal. Biochem. 105 (1980), 389-397). The reaction mixture (10 uL) was transferred into 90 μL of ABTS/GOX/HRP solution (2.74 mg/mL ABTS, 0.1 U/mL HRP, and 1 U/mL GOX). Absorbance at 405 nm was immediately measured at 11 seconds intervals for 5 min using a SoftMax Pro plate reader (Molecular Device). The output was the reaction rate, Vo, and it was used to measure the corn starch hydrolyzing activity of the hybrid glucoamylase variants. As shown in Table 8, all the hybrid glucoamylase variants showed higher corn starch hydrolyzing activity when compared to enzyme constructs lacking a CBM region at either N or C-termini. Moreover, all the hybrid GAs outperformed a commercially relevant Trichoderma reesei variant glucoamylase (previously described in WO2009/067218).

TABLE 8
Corn starch hydrolyzing activity of hybrid variants of various
Mucorales-clade glucoamylases measured as glucose release.

| | | Reaction rate |
|---|---|---|
| Gene structure | Sample name | (Vₒ) |
| CRC24662-WT | SobGA1 | 46.1 |
| CRC24662v2 | SobGA1v2 | 45.4 |
| CRC24662v2_19687_CBM21 | SobGA1v4 | 96.6 |
| CRC24662v2_13978_CBM21 | SobGA1v5 | 91.1 |
| CRC24662v2_AnGA_CBM20 | SobGA1v6 | 78.5 |
| CRC24662v2_TrGA_CBM20 | SobGA1v7 | 79.5 |
| CRC24663-WT | AosGA3 | 48.2 |
| CRC24663v2 | AosGA3v2 | 47.7 |
| CRC24663v2_19687_CBM21 | AosGA3v3 | 101.0 |
| CRC24663v2_13978_CBM21 | AosGA3v4 | 103.3 |
| CRC24663v2_AnGA_CBM20 | AosGA3v5 | 101.1 |
| CRC24663v2_TrGA_CBM20 | AosGA3v6 | 101.4 |
| CRC24664-WT | AelGA1 | 45.6 |
| CRC24664v2 | AelGA1v2 | 46.5 |
| CRC24664v2_19687_CBM21 | AelGA1v3 | 101.0 |
| CRC24664v2_13978_CBM21 | AelGA1v4 | 83.2 |
| CRC24664v2_AnGA_CBM20 | AelGA1v5 | 111.7 |
| CRC24664v2_TrGA_CBM20 | AelGA1v6 | 110.4 |
| CRC24665-WT | AvaGA1 | 49.0 |
| CRC24665v2 | AvaGA1v2 | 49.1 |
| CRC24665v2_19687_CBM21 | AvaGA1v3 | 112.9 |
| CRC24665v2_13978_CBM21 | AvaGA1v4 | 114.0 |
| CRC24665v2_AnGA_CBM20 | AvaGA1v5 | 108.1 |
| CRC24665v2_TrGA_CBM20 | AvaGA1v6 | 108.8 |
| CRC24666-WT | AtrGA1 | 48.7 |
| CRC24666v2 | AtrGA1v2 | 51.9 |
| CRC24666v2_19687_CBM21 | AtrGA1v3 | 106.7 |
| CRC24666v2_13978_CBM21 | AtrGA1v4 | 104.7 |
| CRC24666v2_AnGA_CBM20 | AtrGA1v5 | 101.2 |
| CRC24666v2_TrGA_CBM20 | AtrGA1v6 | 101.5 |
| Benchmark | TrGA variant | 67.5 |

What is claimed is:

1. A hybrid polypeptide comprising:
a) a first amino acid sequence comprising a catalytic module having glucoamylase activity or a functional fragment thereof; and
b) a second amino acid sequence comprising a carbohydrate-binding module (CBM) or a functional fragment thereof,
wherein i) the first amino acid sequence comprising a catalytic module having glucoamylase activity or a functional fragment thereof is derived from Zygomycetes; and ii) the second amino acid sequence is located at the N- and/or C-terminus of the first amino acid sequence.

2. The hybrid polypeptide of claim 1, wherein the first amino acid sequence has at least about 60%, at least about 65%, at least about 70%, at least about 75%, at least about 80%, at least about 85%, at least about 90%, at least about 95%, at least about 97%, at least about 98%, at least about 99%, or about 100% sequence identity to the amino acid sequence of SEQ ID NO: 3, SEQ ID NO: 33, SEQ ID NO: 41, SEQ ID NO:47, SEQ ID NO:49, SEQ ID NO: 53, SEQ ID NO: 55, SEQ ID NO:59, SEQ ID NO: 61, SEQ ID NO: 65, SEQ ID NO: 67, SEQ ID NO: 71, SEQ ID NO: 73, SEQ ID NO: 105, SEQ ID NO: 110, SEQ ID NO: 115, SEQ ID NO: 120, or SEQ ID NO: 125.

3. The hybrid polypeptide of claim 1 or 2, wherein the carbohydrate-binding module belongs to a carbohydrate binding module family selected from the group consisting of CBM20, CBM21, CBM25, CBM26, CBM34, CBM41 and CBM45.

4. The hybrid polypeptide of claim 3, wherein the polypeptide comprises a CBM at the N- and C-terminus of the first amino acid sequence.

5. The hybrid polypeptide of claim 3 or claim 4, wherein the second amino acid sequence has at least about 70%, at least about 75%, at least about 80%, at least about 85%, at least about 90%, at least about 95%, at least about 97%, at least about 98%, at least about 99%, or about 100% sequence identity to one or more amino acid sequence selected from the group consisting of amino acids 25-130 of SEQ ID NO: 1, 24-130 of SEQ ID NO: 5, 22-130 of SEQ ID NO: 7, 24-130 of SEQ ID NO: 9, 24-130 of SEQ ID NO: 11, 22-130 of SEQ ID NO: 13, 25-130 of SEQ ID NO: 15, 26-131 of SEQ ID NO: 17, 25-130 of SEQ ID NO: 19, 24-127 of SEQ ID NO: 21, 23-145 of SEQ ID NO: 23, 28-131 of SEQ ID NO: 25, SEQ ID NO: 31, and SEQ ID NO: 39.

6. The hybrid polypeptide of any one of claims 1-5, further comprising a linker region between the first and second amino acid sequences.

7. The hybrid polypeptide of any one of claims 1-6, further comprising an oligopeptide having at least about 90%, at least about 95%, at least about 97%, at least about 98%, at least about 99%, or about 100% sequence identity to the amino acid sequence of SEQ ID NO: 27 or SEQ ID NO: 35 at the C terminus of the first amino acid sequence.

8. The hybrid polypeptide of any one of claims 1-7, wherein the hybrid polypeptide has an amino acid sequence at least about 60%, at least about 65%, at least about 70%, at least about 75%, at least about 80%, at least about 85%, at least about 90%, at least about 95%, at least about 97%, at least about 98%, at least about 99%, or about 100% sequence identity to the amino acid sequence of SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 106, SEQ ID NO: 107, SEQ ID NO: 108, SEQ ID NO: 109, SEQ ID NO: 111, SEQ ID NO: 112, SEQ ID NO: 113, SEQ ID NO: 114, SEQ ID NO: 116, SEQ ID NO: 117, SEQ ID NO: 118, SEQ ID NO: 119, SEQ ID NO: 121, SEQ ID NO: 122, SEQ ID NO: 123, SEQ ID NO: 124, SEQ ID NO: 126, SEQ ID NO: 127, SEQ ID NO: 128, or SEQ ID NO: 129.

9. The hybrid polypeptide of any one of claims 1-8, wherein the first amino acid sequence lacks a CBM.

10. The hybrid polypeptide of any one of claims 1-9, wherein the first amino acid sequence comprises SEQ ID NO:3.

11. The hybrid polypeptide of claim 10, further comprising an amino acid substitution at position 58 of SEQ ID NO:3.

12. The hybrid polypeptide of claim 11, wherein the amino acid substitution is selected from the group consisting of V58P, V58G, V58A, V58L, V58I, V58F, V58Y, V58W, V58S, V66T, V58C, V58M, V58N, V58Q, V58D, V58E, V58K, V58R, and V58H.

13. A polynucleotide encoding the hybrid polypeptide of any one of claims 1 to 12.

14. A vector comprising the polynucleotide of claim 13.

15. A recombinant host cell comprising the polynucleotide of claim 13 or the vector of claim 14.

16. The recombinant host cell of claim 15, which is an ethanologenic microorganism.

17. The recombinant host cell of claim 15 or claim 16, which is a yeast cell.

18. The recombinant host cell of any one of claims 15-17, which further expresses and secretes one or more additional enzymes selected from the group consisting of protease, hemicellulase, cellulase, peroxidase, lipolytic enzyme, metallolipolytic enzyme, xylanase, lipase, phospholipase, esterase, perhydrolase, cutinase, pectinase, pectate lyase, mannanase, keratinase, reductase, nuclease, oxidase, phenoloxidase, lipoxygenase, ligninase, alpha-amylase, pullulanase, phytase, tannase, pentosanase, malanase, beta-glucanase, arabinosidase, hyaluronidase, chondroitinase, laccase, transferrase, and a combination thereof.

19. A method of producing a hybrid polypeptide in a host cell comprising culturing the host cell of any one of claims 15-18 under conditions suitable for the expression and production of the hybrid polypeptide.

20. The method of claim 19, further comprising recovering the hybrid polypeptide from the culture.

21. An enzyme composition comprising the hybrid polypeptide of any one of claims 1 to 12.

22. The enzyme composition of claim 21, wherein the enzyme composition is used in a starch conversion process.

23. A method for saccharifying a starch-containing material comprising the steps of: i) contacting the starch-containing material with an alpha-amylase; and ii) contacting the starch-containing material with a hybrid polypeptide of any one of claims 1 to 12; wherein the method produces at least 70% free glucose from the starch-containing material (substrate).

24. The method of claim 23, wherein the method includes sequentially or simultaneously performing step (i) and step (ii).

25. The method of claim 23 or claim 24, wherein the method further comprises a pre-saccharification before saccharification step ii).

26. The method of any one of claims 23-25, wherein the step (i) is carried out at or below the gelatinization temperature of the starch-containing material.

27. A saccharide produced by method of any one of claims 23-26.

28. A method for producing fermentation products from the saccharide of claim 27, wherein the saccharide is fermented by a fermenting organism.

29. The method of claim 28, wherein the fermentation process is performed sequentially or simultaneously with the step (ii).

30. The method of claim 28 or claim 29, wherein the fermentation product comprises ethanol.

31. The method of claim 28 or claim 29, wherein the fermentation product comprises a non-ethanol metabolite.

32. The method of claim 31, wherein the metabolite is citric acid, lactic acid, succinic acid, monosodium glutamate, gluconic acid, sodium gluconate, calcium gluconate, potassium gluconate, an organic acid, glucono delta-lactone, sodium erythorbate, omega 3 fatty acid, butanol, iso-butanol, an amino acid, lysine, tyrosine, threonine, glycine, itaconic acid, 1,3-propanediol, vitamins, enzymes, hormones, isoprene or other biochemicals or biomaterials.

33. A method of applying the hybrid polypeptide of any one of claims 1 to 12 in brewing.

34. A method of applying the hybrid polypeptide of any one of claims 1 to 12 to produce beer or a malt-based beverage.

35. A method for increasing starch digestibility in an animal which comprises adding at least one hybrid polypeptide of any one of claims 1 to 12 as a feed additive to feed for an animal.


SEQUENCE LISTING (154 sequences)

SEQ ID NO: 1 - protein, 168 aa, Mucor cordense
MKFSKLFTKPTLFIVSIIISVASSETVPTTANVLVKSYTWDGATLSGQIYIKNLAYAKVVSVIYSDANDNWNNNGNKVAASYSAGIDGTNYEYWTFSGAVSGIKQFYVKYDVSGTSYYDNNGTKNYQVTKTSTTTTAATTTTTATTTTGGTGTTTTTTATATATSTDF

SEQ ID NO: 2 - DNA, 504 bp, Mucor cordense
  source 1..504: mol_type = other DNA
atgaagttcagcaagctgttcaccaagcctaccctgttcatcgtttctattattatcagcgttgcttctagcgagaccgtccctaccaccgctaacgtcctcgttaagtcttacacctgggacggcgctaccctgtctggccagatttacattaagaacctcgcttacgccaaggttgttagcgttatttactctgacgctaacgacaactggaacaacaacggcaacaaggttgccgcttcttactctgctggcattgacggcaccaactacgagtactggaccttcagcggcgccgtttccggcatcaagcagttctacgtcaagtacgacgtcagcggcacctcttactacgacaacaacggcaccaagaactaccaggttaccaagacctctaccaccaccaccgccgctaccaccaccaccaccgctaccaccaccaccggcggcaccggcaccaccaccaccaccaccgccaccgccaccgctaccagcaccgacttc

SEQ ID NO: 3 - protein, 441 aa, Saksenaea vasiformis
PTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 4 - DNA, 1323 bp, Saksenaea vasiformis
  source 1..1323: mol_type = other DNA
cctaccggcaacagcaccattagctcttgggtcagcaagcaggaggacatcagcttcagcgagatgctccgaaacgtcaaccctgagggcaccgccaagggcttcgttgccgcgtcgctcagcaccgctggccccgactacttctacacctggacccgagacgccgctctcgtttcccgagttattgcttacaagtacaacaccaccaacgctggcgactctaagatccacggcgttctggacgactacgttaacttccagattaacacccagtctgagcccaccccttgcaactgcctgggcgagcctaagttcaaccctgacggcagctctttcaccggcccttggggccgcccccagaacgacggccccgctgagcgagcctccagtttcatgctgattgccgactctttcctcagccagaccaagaacgcctcttacttcaccaacaccctcaagcctgccatttacaaggacctcgactacgtcgttgacacctggtctaacccttgcttcgacctgtgggaggaggttaacggcatccacttctacaccctcatggttatgcgacgatctctcctcgacggcgctaacttcgccacccgaaacggcgacaactccaaggcctcaacctactccggcgtcgccgccaagattcaggctcgactgaactctttctgggacgccggcaagaactacattaccgttacccaggactacaagaacggcgttgagaagcctagcggcctcgacgttagcaccctcattgctgctaacgtcgctggcatgggcgacggcttctacacccccggcagcgagcgcatcctcgctaccgctctggctttcgagaagagcatggcaagtctgtaccctctgaacaacaacctgcctagccacctgggcaacgccattggccgataccccgaggacacctacaacggcaacggcaactcccagggcaacccttggttcctcgctaccaccgctttcaccgagctgtactaccgcgccattctggagtggaagaacaccggcgttaccgtcacccctatttctaaggacttcttcgtccgattcgacagcagcgctgctcctggcaagaagtacaaccccggctcccaggagttcgctaccctgacccagtctattgctgccgccgctgaccgattcatgtccaccgtccagtaccaccagaaccctaacggctctctgtctgaggagttcgaccgatctagcggctacatgaccggcgcccgagacctgacctggagtcacgctgctttcattaccgctgctcaggcccgagctggctctccttctttc

SEQ ID NO: 5 - protein, 164 aa, Backusella circina
MKLFGTLKASFVFLIGCIVFAMADTVPTTQVKLQSYTYSGGVLSGTIYVQNIDYTKVVTVIYSDGSGNWNNNGNTISASYSSSISGTNYEYWTFSASVSSIQQFYISYLVDGTTYYDNNGGYGANYAVTATTTTASSTTKTTSGTTKTSTTSTATATSTGTSSF

SEQ ID NO: 6 - DNA, 492 bp, Backusella circina
  source 1..492: mol_type = other DNA
atgaagctgttcggcaccctgaaggcttctttcgttttcctgattggctgcattgttttcgctatggccgacaccgtccctaccacccaggttaagctgcagtcttacacctactctggcggcgtcctgtctggcaccatctacgtccagaacattgactacaccaaggttgttaccgttatatattccgacggctctggcaactggaacaacaacggcaacaccattagcgcttcttacagcagctccatttctggcaccaactacgagtactggaccttctctgccagcgtcagctctattcagcagttctacatctcttacctcgttgacggcaccacctactacgacaacaacggcggctacggcgctaactacgccgttaccgccaccaccaccaccgcttcttccaccaccaagaccacctccggcaccaccaagacctccaccaccagcaccgctaccgccaccagcaccggcacctcttccttc

SEQ ID NO: 7 - protein, 162 aa, Dichotomocladium elegans
MKLNSVWKAFSILLMATMAFAATVPKTQVKLEAYTYSDSIFSGRIFVQNIDYTKIVTVYWSDASNKWDSSKYYTEAAYTHSIPGTNYEYWDFSATIGPSGIKQFYISYQVRGVTYYDNNGGYGVNYDVISSPPTTSDATTIPTTTTSAIPSSTTTIPASTGV

SEQ ID NO: 8 - DNA, 486 bp, Dichotomocladium elegans
  source 1..486: mol_type = other DNA
atgaagctgaactccgtttggaaggctttcagcattctgctcatggctactatggctttcgctgctaccgtccccaagacccaggttaagttagaggcttacacctactctgactctattttctctggccgaattttcgtccagaacatcgactacaccaagattgttaccgtttactggtctgacgcctctaacaagtgggacagctctaagtactacaccgaggccgcttacacccacagcatccccggcaccaactacgagtactgggacttcagcgctaccattggccctagcggcatcaagcagttctacatctcttaccaggtccgaggcgtcacctactacgacaacaacggcggctacggcgtcaactacgacgtcatctccagccctcctaccacctctgacgccaccaccattcctaccaccaccacctccgctattcctagctctaccaccaccatccctgccagcaccggcgtc

SEQ ID NO: 9 - protein, 164 aa, Fennellomyces sp.
MRPSLVWKSLLMVVLTAAMGTYAQTVPSVPIKLESYTYSDNVFAGRIFIRNIAYTKVVKVFWSDESGNWNGNGNYVDASYSGSISGTNYEYWEFSTTIGSAGISQSYLRYDVSGSTYYDNNGNANYDIEEDSTPTPTTTTTSTTTITTTTTSGTSTPTSVPTEV

SEQ ID NO: 10 - DNA, 492 bp, Fennellomyces sp.
  source 1..492: mol_type = other DNA
atgcgccctagcctcgtttggaagtctctcctgatggttgtcctgaccgctgcaatgggcacctacgcccagaccgtccctagcgttcctattaagttagaatcttacacctactctgacaacgttttcgctggccgaattttcatccgaaacattgcttacaccaaggttgtcaaggttttctggtctgacgagtccggcaactggaacggcaacggcaactacgttgacgcctcttactctggctccatctccggcaccaactacgagtactgggagttcagcaccaccattggctctgccggcatctcccagtcttacctgcgatacgacgtcagcggctctacctactacgacaacaacggcaacgctaactacgacattgaggaggacagcacccctacccctaccaccaccaccacctctaccaccaccatcaccaccaccaccacctccggcacctctacccctacctctgttcctaccgaggtc

SEQ ID NO: 11 - protein, 172 aa, Phascolomyces articulosus
MKPIHLWHYLFLAMTAAFMGVLADVPTSANIELDYYTYKDKVFSGRIYVKNIAYEKTVAVYWSDASGDWNNNGNNVAASYSESISGTDYEYWDFSTTIGSGGIKQSYLKYTVSGNTYYDNNGSNNYDITETTATTTTTTTTTTTTGSTATTSTTSGPTSTTSTTPGPTPTNV

SEQ ID NO: 12 - DNA, 516 bp, Phascolomyces articulosus
  source 1..516: mol_type = other DNA
atgaagcctattcacctgtggcactacctgttcctcgccatgaccgctgccttcatgggcgtcctcgctgacgtccctacctccgccaacattgaactcgactactacacctacaaggacaaggttttctccggccgaatttacgtcaagaacatcgcttacgaaaaaaccgttgctgtttactggtctgacgctagtggcgactggaacaacaacggcaacaacgttgccgcttcttactctgagtccatctccggcaccgactacgagtactgggacttcagcaccaccattggctctggcggcatcaagcagtcttacctcaagtacaccgttagcggcaacacctactacgacaacaacggctctaacaactacgacattaccgagaccaccgctaccaccaccaccaccaccaccaccaccaccaccaccggctctaccgccaccacctctaccacctctggccctaccagcaccacctctaccacccccggccctacccctaccaacgtt

SEQ ID NO: 13 - protein, 168 aa, Syncephalastrum racemosum
MQLNLYWKSLAVLALIQMVMAATVPTTQVQLDYYTYSNNVLSGRIYVQNIAYSKVVKVIYSDASGNWNNNGNTISASYVESISGTNYEYWDFSATIGTAGIKQFYLRYDVSGSTYYDNNGGDNNNYNVVATTSTTSSSTTTTTATTTTATKTTSTTSATATPTSSATF

SEQ ID NO: 14 - DNA, 504 bp, Syncephalastrum racemosum
  source 1..504: mol_type = other DNA
atgcagctgaacctgtactggaagagcctcgccgtcctcgctctgattcagatggttatggctgctaccgtccctaccacccaggtccagctcgactactacacctactctaacaacgtcctgtctggccgaatctacgttcagaacattgcttacagcaaggttgttaaggttatctatagcgacgcctccggcaactggaacaacaacggcaacaccatctccgcttcttacgtcgagtctatttccggcaccaactacgagtactgggacttcagcgccaccattggcaccgctggcatcaagcagttctacctgcgatacgacgtttccggctccacctactacgacaacaacggcggcgacaacaacaactacaacgttgttgctaccacctctaccacctctagctctaccaccaccaccaccgctaccaccaccaccgccaccaagaccaccagcaccacctctgccaccgctacccctacctctagcgccaccttc

SEQ ID NO: 15 - protein, 157 aa, Choanephora cucurbitarum
MVSFNFLKKPVFIVITCLTVSVCAQSVPTSDPVKVKTFSYDGNSFSGQIYIKNIAYEKTVTVIYSDGTGNWNNNNNKIAAAFSEAISGSNYEYWTFSASVPSIKQFYVKYDVSGKTYYDNNGSKDYNVVTSGPTTTSSGPSKTTTNGPAPTSTNTNF

SEQ ID NO: 16 - DNA, 471 bp, Choanephora cucurbitarum
  source 1..471: mol_type = other DNA
atggtttctttcaacttcctgaagaagcctgttttcattgttatcacctgcctcaccgtcagcgtttgcgcccagtctgttcctaccagcgaccccgttaaggttaagaccttctcttacgacggcaactctttctctggccagatttacatcaagaacattgcttacgagaagaccgttaccgttatttactccgacggcaccggcaactggaacaacaacaacaacaagattgccgctgctttcagcgaggccatctccggctctaactacgagtactggaccttcagcgctagcgttcctagcatcaagcagttctacgtcaagtacgacgtttccggcaagacctactacgacaacaacggctctaaggactacaacgtcgttaccagcggccctaccaccaccagctctggccctagcaagaccaccaccaacggccccgctcctaccagcaccaacaccaacttc

SEQ ID NO: 17 - protein, 168 aa, Rhizopus stolonifer
MKLFGLPLKPSLFFVVSYLSFLASAASIPSSSEVQLDTYAYDGAKFSGKIYVKNIAYEKTVTVVYADGSDNWNNNGNIISATFSTPISGSNYEYWTFSSSISGIKEFYIKYVVSGKTYYDNNGTKNYQVSTTSSTTTASTTTATRTTTAGTTSTSTTAAPTSTSSGSF

SEQ ID NO: 18 - DNA, 504 bp, Rhizopus stolonifer
  source 1..504: mol_type = other DNA
atgaagctgttcggcctgcccctcaagcctagcctgttcttcgtcgtctcttacctgtctttcctcgcttctgctgccagcatccctagcagcagcgaggtccagctcgacacctacgcttacgacggcgccaagttctctggcaagatttacgtcaagaacattgcttacgagaagaccgttaccgttgtttacgctgacggctctgacaactggaacaacaacggcaacattattagcgctaccttcagcacccctatttccggctctaactacgagtactggaccttcagctctagcattagcggcattaaggagttctacatcaagtacgtcgtttccggcaagacctactacgacaacaacggcaccaagaactaccaggtttccaccaccagctctaccaccaccgctagcaccaccaccgctacccgaaccaccaccgctggcaccaccagcacctctaccaccgctgcccctacctctacctccagcggctccttc

SEQ ID NO: 19 - protein, 167 aa, Mucor circinelloides f. circinelloides
MKFSNLLKKPLLLIAGILAATVVAETVPTTAAVKVKSFTYDGSTFAGQIYVKNIAYTKTVTVIYSDASNNWNNNGNTIAASYSEGISGTNYEYWTFSAPVSGIKQFYVKYVVSGTTYYDNNNSGNYQVTTTTTTTAPTSTTSGGSSTTTGGSTTTATSVPTGVPSGF

SEQ ID NO: 20 - DNA, 501 bp, Mucor circinelloides f. circinelloides
  source 1..501: mol_type = other DNA
atgaagttctctaacctcctgaagaagcccctcctgctcattgctggcattctcgctgccaccgtcgttgccgagaccgtccctaccaccgctgctgttaaggttaagtctttcacctacgacggcagcaccttcgctggccagatttacgtcaagaacatcgcttacaccaagaccgttaccgttatttactctgacgcctctaacaactggaacaacaacggcaacaccattgccgcctcttacagcgagggcatctctggcaccaactacgagtactggaccttcagcgcccctgtttccggcatcaagcagttctacgtcaagtacgttgtcagcggcaccacctactacgacaacaacaactctggcaactaccaggttaccaccaccaccaccaccaccgcccctaccagcaccacctccggcggctctagcaccaccaccggcggctccaccaccaccgccacctctgtccctaccggcgtccctagcggcttc

SEQ ID NO: 21 - protein, 171 aa, Dactylellina haptotyla
MRFPNILKYFAFALAYLYCSVHASVLLRRAGEVTLSSYSYSNNVLSGSITVQNIAYQKVVAVYWAQGSNWQSTPISASYSSGPNSSGYETWTFSGTAVGATQLYIKYDVSGTSYYYPGNYVNVQVTSSATTTTTTSRTTTTTIRTTSSGTTTAGTTTTTTLATSTTATSST

SEQ ID NO: 22 - DNA, 513 bp, Dactylellina haptotyla
  source 1..513: mol_type = other DNA
atgcgattccctaacatcctgaagtacttcgctttcgccctcgcttacctgtactgcagcgttcacgcttccgtcctgctgcgacgcgccggcgaggttaccctgagctcttactcctacagcaacaacgtcctgtctggctccattaccgtccagaacattgcctaccagaaggtcgttgctgtttactgggctcagggctctaactggcagtctacccctatttctgcttcttacagcagcggccccaactcttccggctacgagacctggaccttctccggcaccgccgttggcgctacccagctgtacatcaagtacgacgtttctggcacctcttactactaccccggcaactacgtcaacgttcaggttacctctagcgctaccaccaccaccaccacctcccgaaccaccaccaccaccattcgaaccacctctagcggcaccaccaccgccggcaccaccaccaccaccaccctcgctacctctaccaccgctacctccagcacc

SEQ ID NO: 23 - protein, 170 aa, Lipomyces starkeyi
MLLINFFIAVLGVISLSPIVVARYILRRDCTTVTVLSSPESVTSSNHVQLASHEMCDSTLSASLYIYNDDYDKIVTLYYLTSSGTTGSVTASYSSSLSNNWELWSLSAPAADAVEITGASYVDSDASATYATSFDIPLTTTTTSSSSASATSTSSLTTTSSVSISVSVPT

SEQ ID NO: 24 - DNA, 510 bp, Lipomyces starkeyi
  source 1..510: mol_type = other DNA
atgctgctcatcaacttcttcattgccgtcctcggcgttatctccctcagccctattgttgttgcccgatacattctgcgccgagactgcaccaccgttaccgttctgagcagccccgagtctgttacctctagcaaccacgttcagctggcttctcacgagatgtgcgactctaccctgtccgctagcctgtacatttacaacgacgactacgacaagatcgtcaccctgtactacctgaccagctccggcaccaccggcagcgtcaccgcttcctactctagcagcctgtccaacaactgggagctgtggagcctgtctgctcccgccgctgacgccgttgagattaccggcgcttcttacgtcgactccgacgcttctgctacctacgctacctctttcgacatccccctgaccaccaccaccacctctagctcttccgccagcgctaccagcacctccagcctgaccaccacctctagcgtctccattagcgtttccgttcctacc

SEQ ID NO: 25 - protein, 164 aa, Rhizopus microsporus var. microsporus
MKLMNSSMKTCVFFILSYFSLLVSSAAVPTSAAVQVESYKYDGTTFSGRIFVKNIAYSKVVTVIYSDGSDNWNNNNNKISAAYSEAISGSNYEYWTFSAKLSGIKQFYVKYEVSGSTYYDNNGTKNYQVQATSATSTTATATTTTSTSTTTTSTGPTSTASVSF

SEQ ID NO: 26 - DNA, 492 bp, Rhizopus microsporus var. microsporus
  source 1..492: mol_type = other DNA
atgaagctcatgaactctagcatgaagacctgcgttttcttcatcctgtcttacttcagcctcctcgtcagcagcgctgccgttcctacctctgctgctgttcaggttgagtcctacaagtacgacggcaccaccttcagcggccgaattttcgtcaagaacattgcttactccaaggttgttaccgtcatatatagcgacggctctgacaactggaacaacaacaacaacaagatcagcgctgcttacagcgaggctattagcggctccaactacgagtactggaccttctctgctaagctgtccggcattaagcagttctacgtcaagtacgaggtttctggctctacctactacgacaacaacggcaccaagaactaccaggtccaggctacctctgccaccagcaccaccgccaccgccaccaccaccacctctaccagcaccaccaccaccagcaccggccctaccagcaccgcaagtgtttctttc

SEQ ID NO: 27 - protein, 34 aa, Aspergillus niger
PASWGETSASSVPGTCAATSAIGTYSSVTVTSWP

SEQ ID NO: 28 - DNA, 102 bp, Aspergillus niger
  source 1..102: mol_type = other DNA
cctgcttcttggggcgagacctccgcttctagcgtccccggcacctgcgctgctaccagcgctattggcacctactctagcgtcaccgttacctcttggcct

SEQ ID NO: 29 - protein, 41 aa, Aspergillus niger
SIVATGGTTTTATPTGSGSVTSTSKTTATASKTSTSTSSTS

SEQ ID NO: 30 - DNA, 123 bp, Aspergillus niger
  source 1..123: mol_type = other DNA
tccattgttgctaccggcggcaccaccaccaccgccacccctaccggctctggctctgttaccagcaccagcaagaccaccgctaccgcttctaagacctccacctctacctctagcaccagc

SEQ ID NO: 31 - protein, 108 aa, Aspergillus niger [sequence omitted]

SEQ ID NO: 32 - DNA, 324 bp, Aspergillus niger
  source 1..324: mol_type = other DNA
tgcaccacccctaccgccgtcgccgttaccttcgacctgaccgctaccaccacctacggcgagaacatttacctggttggctccattagccagctgggcgactgggagacctccgacggcatcgccctgtctgctgacaagtacaccagctccgaccctctgtggtacgttaccgttaccctgcccgccggcgagtctttcgagtacaagttcatccgaattgagtctgacgactctgttgagtgggagagcgaccccaaccgagagtacaccgttcctcaggcttgcggcaccagcaccgctaccgtcaccgacacctggcgc

SEQ ID NO: 33 - protein, 466 aa, Saksenaea vasiformis
MPSWKTLFLLLGPIATAAAAPVDKQSLPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSP

SEQ ID NO: 34 - DNA, 1398 bp, Saksenaea vasiformis
  source 1..1398: mol_type = other DNA
atgccttcttggaaaaccctgttcctcctcctgggccctatcgccaccgctgccgctgctcccgttgacaagcagtctctgcctaccggcaacagcaccattagctcttgggtcagcaagcaggaggacatcagcttcagcgagatgctccgaaacgtcaaccctgagggcaccgccaagggcttcgttgccgcgtcgctcagcaccgctggccccgactacttctacacctggacccgagacgccgctctcgtttcccgagttattgcttacaagtacaacaccaccaacgctggcgactctaagatccacggcgttctggacgactacgttaacttccagattaacacccagtctgagcccaccccttgcaactgcctgggcgagcctaagttcaaccctgacggcagctctttcaccggcccttggggccgcccccagaacgacggccccgctgagcgagcctccagtttcatgctgattgccgactctttcctcagccagaccaagaacgcctcttacttcaccaacaccctcaagcctgccatttacaaggacctcgactacgtcgttgacacctggtctaacccttgcttcgacctgtgggaggaggttaacggcatccacttctacaccctcatggttatgcgacgatctctcctcgacggcgctaacttcgccacccgaaacggcgacaactccaaggcctcaacctactccggcgtcgccgccaagattcaggctcgactgaactctttctgggacgccggcaagaactacattaccgttacccaggactacaagaacggcgttgagaagcctagcggcctcgacgttagcaccctcattgctgctaacgtcgctggcatgggcgacggcttctacacccccggcagcgagcgcatcctcgctaccgctctggctttcgagaagagcatggcaagtctgtaccctctgaacaacaacctgcctagccacctgggcaacgccattggccgataccccgaggacacctacaacggcaacggcaactcccagggcaacccttggttcctcgctaccaccgctttcaccgagctgtactaccgcgccattctggagtggaagaacaccggcgttaccgtcacccctatttctaaggacttcttcgtccgattcgacagcagcgctgctcctggcaagaagtacaaccccggctcccaggagttcgctaccctgacccagtctattgctgccgccgctgaccgattcatgtccaccgtccagtaccaccagaaccctaacggctctctgtctgaggagttcgaccgatctagcggctacatgaccggcgcccgagacctgacctggagtcacgctgctttcattaccgctgctcaggcccgagctggctctcct

SEQ ID NO: 35 - protein, 33 aa, Trichoderma reesei
PSWANSSASTIPSTCSGASVVGSYSRPTATSFP

SEQ ID NO: 36 - DNA, 99 bp, Trichoderma reesei
  source 1..99: mol_type = other DNA
ccttcttgggctaacagctctgcttctaccattcctagcacctgcagcggcgctagcgtcgttggctcttactcccgacctaccgctacctctttccct

SEQ ID NO: 37 - protein, 19 aa, Trichoderma reesei
PSQTPKPGVPSGTPYTPLP

SEQ ID NO: 38 - DNA, 57 bp, Trichoderma reesei
  source 1..57: mol_type = other DNA
ccttctcagacccctaagcccggcgttcctagcggcaccccttacacccctctgcct

SEQ ID NO: 39 - protein, 109 aa, Trichoderma reesei
CATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 40 - DNA, 327 bp, Trichoderma reesei
  source 1..327: mol_type = other DNA
tgcgccacccctaccagcgtcgccgttaccttccacgagctcgtttctacccagttcggccagaccgtcaaggttgctggcaacgccgctgccctgggcaactggagcaccagcgccgctgttgctctcgacgctgttaactacgctgacaaccaccctctgtggattggcaccgttaacttagaagccggcgacgttgttgagtacaagtacatcaacgtcggccaggacggctccgttacctgggagtccgaccccaaccacacctacaccgttcctgctgtcgcttgcgtcacccaggttgtcaaggaggacacctggcagtct

SEQ ID NO: 41 - protein, 466 aa, Saksenaea vasiformis
MPSWKTLFLLLGPIATAAAAPVDKQSLPTGNSTISSWVSKQEDISFSEMLRNVNPEGTAKGFVAASLSTAGPDYFYTWTRDAALVSRVIAYKYNTTNAGDSKIHGVLDDYVNFQINTQSESTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSP

SEQ ID NO: 42 - DNA, 1398 bp, Saksenaea vasiformis
  source 1..1398: mol_type = other DNA
atgccttcttggaaaaccctgttcctcctcctgggccctatcgccaccgctgccgctgctcccgttgacaagcagtctctgcctaccggcaacagcaccattagctcttgggtcagcaagcaggaggacatcagcttcagcgagatgctccgaaacgtcaaccctgagggcaccgccaagggcttcgttgccgcgtcgctcagcaccgctggccccgactacttctacacctggacccgagacgccgctctcgtttcccgagttattgcttacaagtacaacaccaccaacgctggcgactctaagatccacggcgttctggacgactacgttaacttccagattaacacccagtctgagtctaccccttgcaactgcctgggcgagcctaagttcaaccctgacggcagctctttcaccggcccttggggccgcccccagaacgacggccccgctgagcgagcctccagtttcatgctgattgccgactctttcctcagccagaccaagaacgcctcttacttcaccaacaccctcaagcctgccatttacaaggacctcgactacgtcgttgacacctggtctaacccttgcttcgacctgtgggaggaggttaacggcatccacttctacaccctcatggttatgcgacgatctctcctcgacggcgctaacttcgccacccgaaacggcgacaactccaaggcctcaacctactccggcgtcgccgccaagattcaggctcgactgaactctttctgggacgccggcaagaactacattaccgttacccaggactacaagaacggcgttgagaagcctagcggcctcgacgttagcaccctcattgctgctaacgtcgctggcatgggcgacggcttctacacccccggcagcgagcgcatcctcgctaccgctctggctttcgagaagagcatggcaagtctgtaccctctgaacaacaacctgcctagccacctgggcaacgccattggccgataccccgaggacacctacaacggcaacggcaactcccagggcaacccttggttcctcgctaccaccgctttcaccgagctgtactaccgcgccattctggagtggaagaacaccggcgttaccgtcacccctatttctaaggacttcttcgtccgattcgacagcagcgctgctcctggcaagaagtacaaccccggctcccaggagttcgctaccctgacccagtctattgctgccgccgctgaccgattcatgtccaccgtccagtaccaccagaaccctaacggctctctgtctgaggagttcgaccgatctagcggctacatgaccggcgcccgagacctgacctggagtcacgctgctttcattaccgctgctcaggcccgagctggctctcct

SEQ ID NO: 43 - DNA, 123 bp, Aspergillus niger
  source 1..123: mol_type = other DNA
tctattgttgctaccggcggcaccaccaccaccgctacccctaccggctccggctctgttaccagcacctccaagaccaccgccaccgcttctaagaccagcaccagcacctcttctaccagc

SEQ ID NO: 44 - DNA, 324 bp, Aspergillus niger
  source 1..324: mol_type = other DNA
tgcaccacccctaccgctgtcgccgttaccttcgacctgaccgctaccaccacctacggcgagaacatttacctggttggctccattagccagctgggcgactgggagacctctgacggcatcgccctgagcgccgacaagtacacctctagcgaccctctgtggtacgtcaccgtcaccctgcctgctggcgagtctttcgagtacaagttcatccgaattgagtctgacgacagcgttgagtgggagtccgaccccaaccgagagtacaccgttcctcaggcttgcggcacctctaccgctaccgttaccgacacctggcgc

SEQ ID NO: 45 - protein, 19 aa, Saksenaea oblongispora
MPSWKTLIFLLGPITAALA

SEQ ID NO: 46 - DNA, 57 bp, Saksenaea oblongispora
  source 1..57: mol_type = other DNA
atgccttcttggaagaccctgattttcctcctgggccctattaccgccgctctggct

SEQ ID NO: 47 - protein, 449 aa, Saksenaea oblongispora
APVNKQSFPTGNPDISSWVSKQEGISFSEMLRNVNPPGTAKGFVAASLSTAGPDYFYTWTRDAALVSRVIAYKYNTTNAGDNNIHGALQDYVTFQINTQTESTPCNCLGEPKFNPDGSSFTGPWGRPQNDGPAERASSMILIADSFLAQTKDTAYVTNTLKPAIYKDLDYVVNTWSNPCFDLWEEVNGVHFYTLMVMRRGLLDGADFATRNGDASKASSYSDAASKIKTRLDSFWVSDKNYITVTQDYKEGVKKDTGLDVSTLIAANVAGKDDGFYTPGSDKILATAVAFENAMAKLYPLNQNLDSHLGNSIGRYPEDTYNGNGNSQGNPWFLATTAFTELYYRAILEWKDSGVTVTPVSKDFFAKFDSNAVAGKKYSPNSEEFATLTANIAAAADRFMSTVKYHQNPNGSLSEQFDRHSGYMTGARDLTWSHAAFITAAQARAGTPSF

SEQ ID NO: 48 - DNA, 1347 bp, Saksenaea oblongispora
  source 1..1347: mol_type = other DNA
gctcctgttaacaagcagtctttccctaccggcaaccccgacatcagctcttgggtttctaagcaggagggcattagcttcagcgagatgctccgaaacgtcaaccctcccggcaccgctaagggcttcgttgctgctagcctcagcaccgccggccccgactacttctacacctggacccgagacgccgctctcgtctcccgcgtcatcgcttacaagtacaacaccaccaacgctggcgacaacaacattcacggcgctctgcaggactacgtcaccttccagattaacacccagaccgagtccaccccttgcaactgcctgggcgagcctaagttcaaccccgacggcagctctttcaccggcccttggggccgacctcagaacgacggccctgctgagcgcgctagcagcatgattctcattgctgacagcttcctcgcccagaccaaggacaccgcttacgttaccaacaccctgaagcctgccatttacaaggacctggactacgttgttaacacctggtctaacccttgcttcgacctgtgggaggaggttaacggcgtccacttctacaccctcatggttatgcgacgaggcctcctcgacggcgctgacttcgctacccgaaacggcgacgcctctaaggctagctcttactctgacgctgctagcaagatcaagacccgactggactctttctgggtcagcgacaagaactacattaccgttacccaggactacaaggagggcgtcaagaaggacaccggcctcgacgttagcaccctcattgctgccaacgttgccggcaaggacgacggcttctacacccccggcagcgacaagattctggctaccgctgttgccttcgagaacgccatggctaagctgtaccctctgaaccagaacctcgacagccacctgggcaactctattggccgataccctgaggacacctacaacggcaacggcaactcccagggcaacccttggttcctcgccaccaccgctttcaccgagctgtactaccgagccatcttggaatggaaggacagcggcgttaccgtcacccccgtttccaaggacttcttcgccaagttcgactccaacgccgttgccggcaagaagtactctcctaactctgaggagttcgccaccctgaccgctaacatcgctgctgccgctgaccgattcatgtctaccgttaagtaccaccagaaccctaacggctctctgtccgagcagttcgaccgccacagcggctacatgaccggcgcccgagacctgacctggtctcacgctgctttcattaccgccgcccaggctcgcgctggcacccctagcttc

SEQ ID NO: 49 - protein, 449 aa, Saksenaea oblongispora
APVNKQSFPTGNPDISSWVSKQEGISFSEMLRNVNPPGTAKGFVAASLSTAGPDYFYTWTRDAALVSRVIAYKYNTTNAGDNNIHGALQDYVTFQINTQTEPTPCNCLGEPKFNPDGSSFTGPWGRPQNDGPAERASSMILIADSFLAQTKDTAYVTNTLKPAIYKDLDYVVNTWSNPCFDLWEEVNGVHFYTLMVMRRGLLDGADFATRNGDASKASSYSDAASKIKTRLDSFWVSDKNYITVTQDYKEGVKKDTGLDVSTLIAANVAGKDDGFYTPGSDKILATAVAFENAMAKLYPLNQNLDSHLGNSIGRYPEDTYNGNGNSQGNPWFLATTAFTELYYRAILEWKDSGVTVTPVSKDFFAKFDSNAVAGKKYSPNSEEFATLTANIAAAADRFMSTVKYHQNPNGSLSEQFDRHSGYMTGARDLTWSHAAFITAAQARAGTPSF

SEQ ID NO: 50 - DNA, 1347 bp, Saksenaea oblongispora
  source 1..1347: mol_type = other DNA
gctcctgttaacaagcagtctttccctaccggcaaccccgacatcagctcttgggtttctaagcaggagggcattagcttcagcgagatgctccgaaacgtcaaccctcccggcaccgctaagggcttcgttgctgctagcctcagcaccgccggccccgactacttctacacctggacccgagacgccgctctcgtctcccgcgtcatcgcttacaagtacaacaccaccaacgctggcgacaacaacattcacggcgctctgcaggactacgtcaccttccagattaacacccagaccgagcccaccccttgcaactgcctgggcgagcctaagttcaaccccgacggcagctctttcaccggcccttggggccgacctcagaacgacggccctgctgagcgcgctagcagcatgattctcattgctgacagcttcctcgcccagaccaaggacaccgcttacgttaccaacaccctgaagcctgccatttacaaggacctggactacgttgttaacacctggtctaacccttgcttcgacctgtgggaggaggttaacggcgtccacttctacaccctcatggttatgcgacgaggcctcctcgacggcgctgacttcgctacccgaaacggcgacgcctctaaggctagctcttactctgacgctgctagcaagatcaagacccgactggactctttctgggtcagcgacaagaactacattaccgttacccaggactacaaggagggcgtcaagaaggacaccggcctcgacgttagcaccctcattgctgccaacgttgccggcaaggacgacggcttctacacccccggcagcgacaagattctggctaccgctgttgccttcgagaacgccatggctaagctgtaccctctgaaccagaacctcgacagccacctgggcaactctattggccgataccctgaggacacctacaacggcaacggcaactcccagggcaacccttggttcctcgccaccaccgctttcaccgagctgtactaccgagccatcttggaatggaaggacagcggcgttaccgtcacccccgtttccaaggacttcttcgccaagttcgactccaacgccgttgccggcaagaagtactctcctaactctgaggagttcgccaccctgaccgctaacatcgctgctgccgctgaccgattcatgtctaccgttaagtaccaccagaaccctaacggctctctgtccgagcagttcgaccgccacagcggctacatgaccggcgcccgagacctgacctggtctcacgctgctttcattaccgccgcccaggctcgcgctggcacccctagcttc

SEQ ID NO: 51 - protein, 20 aa, Apophysomyces ossiformis
MMMSWKSLLLLLGPIAAATA

SEQ ID NO: 52 - DNA, 60 bp, Apophysomyces ossiformis
  source 1..60: mol_type = other DNA
atgatgatgtcttggaagtctctgctcctcctgctgggccctatcgccgctgctaccgct

SEQ ID NO: 53 - protein, 443 aa, Apophysomyces ossiformis
AIPTGNSSLNAWVSSQEDISFSVMLGNINPPGTVKGFVAASLSTAGPDYFYSWVRDSALVSRVVTHKYNTTETGNSTVAGFLEDYVHFQINTQTESTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSNKNYIIVTQDYQNGVQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSNEFASLAQNIASAADRFLSTVNYHRNSNGSLSEEFDRNTGYMTGARDLTWSHAAFITAGLARAGTPSF

SEQ ID NO: 54 - DNA, 1329 bp, Apophysomyces ossiformis
  source 1..1329: mol_type = other DNA
gccatccctaccggcaacagcagcctgaacgcttgggttagctcccaggaggacatctctttctccgtcatgctgggcaacattaaccctcccggcaccgtcaagggcttcgttgccgctagcctgtccaccgctggccccgactacttctactcttgggtccgagacagcgccctcgtttcccgcgtcgtcacccacaagtacaacaccaccgagaccggcaactctaccgttgccggcttcttggaggactacgttcacttccagattaacacccagaccgagtctaccgtttgcaactgcctgggcgagcctaagttcaaccccgacggcagctcttacaccggcccttggggccgccctcagaacgacggccccgctgagcgagctagcaccatgattctgattgccgactctttcctgacccagaccaagaacacctcttacgttgacaacaccctgaagcccgccatttacaaggacctggactacgttgttaacacctggtctaacccttgcttcgacctgtgggaggaggttaacggcattcacttctacaccctcatggttatgcgacgaggcctcctcgacggcgccaacttcgccacccgaaacggcgacacctctaaggctagctcttacagcagcaccgctagccagattcagaacaagattgactccttctggagctccaacaagaactacattattgttacccaggactaccagaacggcgtccagaagcctagcggcctcgacatcagcaccctcattgctgccaacgtcgctggcatgaacgacggcttctacacccccggctctgacaagatgctcgccaccgccgttgctattgagaacgccatggctaacctgtaccccctcaacaagaacctgccttcttacctgggcaacgctattggccgataccctgaggacacctacaacggcgacggcaactcccagggcaacccttggttcctcgccaccaccgctttcagcgagctgtactaccgagctctgctggagtggcaggagaccggcgttaccgttaccagcatttctaaggacttcttcagcaagttcgaccctaacgctagcgctggcaagaagtactctcccggctctaacgagttcgcttctctggctcagaacattgctagcgctgctgaccgattcctgtccaccgtcaactaccaccgaaactccaacggcagcctgtctgaggagttcgaccgaaacaccggctacatgaccggcgcccgagacctgacctggtctcacgccgctttcattaccgctggcctcgctcgcgctggcacccctagcttc

SEQ ID NO: 55 - protein, 443 aa, Apophysomyces ossiformis
AIPTGNSSLNAWVSSQEDISFSVMLGNINPPGTVKGFVAASLSTAGPDYFYSWVRDSALVSRVVTHKYNTTETGNSTVAGFLEDYVHFQINTQTEPTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSNKNYIIVTQDYQNGVQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSNEFASLAQNIASAADRFLSTVNYHRNSNGSLSEEFDRNTGYMTGARDLTWSHAAFITAGLARAGTPSF

SEQ ID NO: 56 - DNA, 1329 bp, Apophysomyces ossiformis
  source 1..1329: mol_type = other DNA
gccatccctaccggcaacagcagcctgaacgcttgggttagctcccaggaggacatctctttctccgtcatgctgggcaacattaaccctcccggcaccgtcaagggcttcgttgccgctagcctgtccaccgctggccccgactacttctactcttgggtccgagacagcgccctcgtttcccgcgtcgtcacccacaagtacaacaccaccgagaccggcaactctaccgttgccggcttcttggaggactacgttcacttccagattaacacccagaccgagcccaccgtttgcaactgcctgggcgagcctaagttcaaccccgacggcagctcttacaccggcccttggggccgccctcagaacgacggccccgctgagcgagctagcaccatgattctgattgccgactctttcctgacccagaccaagaacacctcttacgttgacaacaccctgaagcccgccatttacaaggacctggactacgttgttaacacctggtctaacccttgcttcgacctgtgggaggaggttaacggcattcacttctacaccctcatggttatgcgacgaggcctcctcgacggcgccaacttcgccacccgaaacggcgacacctctaaggctagctcttacagcagcaccgctagccagattcagaacaagattgactccttctggagctccaacaagaactacattattgttacccaggactaccagaacggcgtccagaagcctagcggcctcgacatcagcaccctcattgctgccaacgtcgctggcatgaacgacggcttctacacccccggctctgacaagatgctcgccaccgccgttgctattgagaacgccatggctaacctgtaccccctcaacaagaacctgccttcttacctgggcaacgctattggccgataccctgaggacacctacaacggcgacggcaactcccagggcaacccttggttcctcgccaccaccgctttcagcgagctgtactaccgagctctgctggagtggcaggagaccggcgttaccgttaccagcatttctaaggacttcttcagcaagttcgaccctaacgctagcgctggcaagaagtactctcccggctctaacgagttcgcttctctggctcagaacattgctagcgctgctgaccgattcctgtccaccgtcaactaccaccgaaactccaacggcagcctgtctgaggagttcgaccgaaacaccggctacatgaccggcgcccgagacctgacctggtctcacgccgctttcattaccgctggcctcgctcgcgctggcacccctagcttc

SEQ ID NO: 57 - protein, 20 aa, Apophysomyces elegans
MMMSWKSLLLLLGPIAAATA

SEQ ID NO: 58 - DNA, 60 bp, Apophysomyces elegans
  source 1..60: mol_type = other DNA
atgatgatgtcttggaagtctctgctcctgctcctgggccctattgctgccgccaccgct

SEQ ID NO: 59 - protein, 442 aa, Apophysomyces elegans
AIPTGNSSLNAWVSSQEDISFSVMLGNINPPGTVKGFVAASLSTAGPDYFYSWVRDSALVSRVVTHKYNTTETGNSTVAGFLQDYVHFQINTQTESTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIMVTQDYQNGIQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSDEFNSLAQNIASAADRFLSTVNYHRNSNGSLSEEYDRNSGYMTGARDLTWSHAAFITAGLARAGTPS

SEQ ID NO: 60 - DNA, 1326 bp, Apophysomyces elegans
  source 1..1326: mol_type = other DNA
gccatccctaccggcaactctagcctcaacgcttgggttagcagccaggaggacatttctttcagcgtcatgctgggcaacattaaccctcccggcaccgtcaagggcttcgttgctgctagcctgtccaccgctggccccgactacttctactcttgggttcgagacagcgctctcgtttctcgcgtcgttacccacaagtacaacaccaccgagaccggcaacagcaccgttgctggcttcctgcaggactacgttcacttccagattaacacccagaccgagtccaccgtttgcaactgcctgggcgagcctaagttcaaccccgacggcagctcttacaccggcccttggggccgccctcagaacgacggccccgctgagcgagctagcaccatgattctgattgctgactccttcctgacccagaccaagaacacctcttacgttgacaacaccctgaagcccgctatttacaaggacctggactacgtcgtcaacacctggtctaacccttgcttcgacctgtgggaggaggttaacggcattcacttctacaccctcatggttatgcgacgaggcctcctggacggcgccaacttcgccacccgaaacggcgacaccagcaaggctagctcttacagcagcaccgcttctcagatccagaacaagattgactctttctggtctagctctaagaactacatcatggttacccaggactaccagaacggcattcagaagcctagcggcctcgacattagcaccctcatcgccgctaacgtcgctggcatgaacgacggcttctacacccccggctccgacaagatgctcgctaccgccgttgctattgagaacgccatggctaacctgtaccctctgaacaagaacctgccttcttacctgggcaacgccattggccgataccccgaggacacctacaacggcgacggcaactctcagggcaacccttggttcctcgccaccaccgctttcagcgagctgtactaccgcgctctcctggagtggcaggagaccggcgttaccgtcaccagcatttccaaggacttcttctctaagttcgaccctaacgctagcgctggcaagaagtactcccccggctctgacgagttcaactccctcgcccagaacattgccagcgctgccgaccgattcctgtctaccgttaactaccaccgaaactccaacggctctctgtctgaggagtacgaccgaaacagcggctacatgaccggcgcccgagacctgacctggtcccacgccgctttcattaccgccggcctcgcgagagctggcacccctagc

SEQ ID NO: 61 - protein, 442 aa, Apophysomyces elegans
AIPTGNSSLNAWVSSQEDISFSVMLGNINPPGTVKGFVAASLSTAGPDYFYSWVRDSALVSRVVTHKYNTTETGNSTVAGFLQDYVHFQINTQTEPTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIMVTQDYQNGIQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSDEFNSLAQNIASAADRFLSTVNYHRNSNGSLSEEYDRNSGYMTGARDLTWSHAAFITAGLARAGTPS

SEQ ID NO: 62 - DNA, 1326 bp, Apophysomyces elegans
  source 1..1326: mol_type = other DNA
gccatccctaccggcaactctagcctcaacgcttgggttagcagccaggaggacatttctttcagcgtcatgctgggcaacattaaccctcccggcaccgtcaagggcttcgttgctgctagcctgtccaccgctggccccgactacttctactcttgggttcgagacagcgctctcgtttctcgcgtcgttacccacaagtacaacaccaccgagaccggcaacagcaccgttgctggcttcctgcaggactacgttcacttccagattaacacccagaccgagcccaccgtttgcaactgcctgggcgagcctaagttcaaccccgacggcagctcttacaccggcccttggggccgccctcagaacgacggccccgctgagcgagctagcaccatgattctgattgctgactccttcctgacccagaccaagaacacctcttacgttgacaacaccctgaagcccgctatttacaaggacctggactacgtcgtcaacacctggtctaacccttgcttcgacctgtgggaggaggttaacggcattcacttctacaccctcatggttatgcgacgaggcctcctggacggcgccaacttcgccacccgaaacggcgacaccagcaaggctagctcttacagcagcaccgcttctcagatccagaacaagattgactctttctggtctagctctaagaactacatcatggttacccaggactaccagaacggcattcagaagcctagcggcctcgacattagcaccctcatcgccgctaacgtcgctggcatgaacgacggcttctacacccccggctccgacaagatgctcgctaccgccgttgctattgagaacgccatggctaacctgtaccctctgaacaagaacctgccttcttacctgggcaacgccattggccgataccccgaggacacctacaacggcgacggcaactctcagggcaacccttggttcctcgccaccaccgctttcagcgagctgtactaccgcgctctcctggagtggcaggagaccggcgttaccgtcaccagcatttccaaggacttcttctctaagttcgaccctaacgctagcgctggcaagaagtactcccccggctctgacgagttcaactccctcgcccagaacattgccagcgctgccgaccgattcctgtctaccgttaactaccaccgaaactccaacggctctctgtctgaggagtacgaccgaaacagcggctacatgaccggcgcccgagacctgacctggtcccacgccgctttcattaccgccggcctcgcgagagctggcacccctagc

SEQ ID NO: 63 - protein, 20 aa, Apophysomyces variabilis
MMMSWKSLLLLLGPIAAATA

SEQ ID NO: 64 - DNA, 60 bp, Apophysomyces variabilis
  source 1..60: mol_type = other DNA
atgatgatgtcttggaagtctctgctcctcctgctgggccctattgccgctgccaccgct

SEQ ID NO: 65 - protein, 443 aa, Apophysomyces variabilis
AIPTGNSSLNAWVSSQEDISFSVMLGNINPPGTVKGFVAASLSTAGPDYFYSWVRDSALVSRVVTHKYNTTETGNSTVAGFLQDYVHFQINTQTESTVCNCLGEPKFNPDGSSYTGPWGRPQNDGPAERASTMILIADSFLAQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIMVTQDYQNGVQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSDEFNSLAQNIASAADRFLSTVNYHRNSNGSLSEEYDRNSGYMTGARDLTWSHAAFITAGLARAGTPSF

SEQ ID NO: 66 - DNA, 1329 bp, Apophysomyces variabilis
  source 1..1329: mol_type = other DNA
gccattcctaccggcaacagcagcctcaacgcttgggttagctcccaggaggacatttctttcagcgtcatgctgggcaacattaaccctcccggcaccgttaagggcttcgttgccgctagcctgtctaccgccggccccgactacttctactcttgggtccgagacagcgccctcgtttcccgcgtcgtcacccacaagtacaacaccaccgagaccggcaacagcaccgttgctggcttcctgcaggactacgtccacttccagattaacacccagaccgagtccaccgtttgcaactgcctgggcgagcctaagttcaaccccgacggcagctcttacaccggcccttggggccgacctcagaacgacggccccgctgagcgcgctagcaccatgattctgattgctgactccttcctcgctcagaccaagaacacctcttacgttgacaacaccctgaagcctgccatttacaaggacctggactacgtcgttaacacctggtctaacccttgcttcgacctgtgggaggaggttaacggcattcacttctacaccctcatggttatgcgacgaggcctcctggacggcgccaacttcgccacccgaaacggcgacacctccaaggctagctcttacagcagcaccgctagccagatccagaacaagattgactctttctggtctagctccaagaactacatcatggttacccaggactaccagaacggcgttcagaagcctagcggcctcgacattagcaccctcatcgccgctaacgttgctggcatgaacgacggcttctacacccctggctctgacaagatgctcgccaccgccgttgctattgagaacgctatggctaacctgtaccctctgaacaagaacctgccttcttacctgggcaacgccattggccgataccccgaggacacctacaacggcgacggcaactcccagggcaacccttggttcctcgctaccaccgctttcagcgagctgtactaccgagctctgttagagtggcaggagaccggcgtcaccgttaccagcatctctaaggacttcttctccaagttcgaccctaacgctagcgccggcaagaagtactcccccggcagcgacgagttcaactctctcgcccagaacatcgccagcgccgctgaccgattcctgtctaccgtcaactaccaccgaaactctaacggctctctgtctgaggagtacgaccgaaactccggctacatgaccggcgcgagagacctgacctggtctcacgctgctttcattaccgctggcctcgcccgagctggcaccccttctttc

SEQ ID NO: 67 - protein, 443 aa, Apophysomyces variabilis
AIPTGNSSLNAWVSSQEDISFSVMLGNINPPGTVKGFVAASLSTAGPDYFYSWVRDSALVSRVVTHKYNTTETGNSTVAGFLQDYVHFQINTQTEPTVCNCLGEPKFNPDGSSYTGPWGRPQNDGPAERASTMILIADSFLAQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIMVTQDYQNGVQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSDEFNSLAQNIASAADRFLSTVNYHRNSNGSLSEEYDRNSGYMTGARDLTWSHAAFITAGLARAGTPSF

SEQ ID NO: 68 - DNA, 1329 bp, Apophysomyces variabilis
  source 1..1329: mol_type = other DNA
gccattcctaccggcaacagcagcctcaacgcttgggttagctcccaggaggacatttctttcagcgtcatgctgggcaacattaaccctcccggcaccgttaagggcttcgttgccgctagcctgtctaccgccggccccgactacttctactcttgggtccgagacagcgccctcgtttcccgcgtcgtcacccacaagtacaacaccaccgagaccggcaacagcaccgttgctggcttcctgcaggactacgtccacttccagattaacacccagaccgagcccaccgtttgcaactgcctgggcgagcctaagttcaaccccgacggcagctcttacaccggcccttggggccgacctcagaacgacggccccgctgagcgcgctagcaccatgattctgattgctgactccttcctcgctcagaccaagaacacctcttacgttgacaacaccctgaagcctgccatttacaaggacctggactacgtcgttaacacctggtctaacccttgcttcgacctgtgggaggaggttaacggcattcacttctacaccctcatggttatgcgacgaggcctcctggacggcgccaacttcgccacccgaaacggcgacacctccaaggctagctcttacagcagcaccgctagccagatccagaacaagattgactctttctggtctagctccaagaactacatcatggttacccaggactaccagaacggcgttcagaagcctagcggcctcgacattagcaccctcatcgccgctaacgttgctggcatgaacgacggcttctacacccctggctctgacaagatgctcgccaccgccgttgctattgagaacgctatggctaacctgtaccctctgaacaagaacctgccttcttacctgggcaacgccattggccgataccccgaggacacctacaacggcgacggcaactcccagggcaacccttggttcctcgctaccaccgctttcagcgagctgtactaccgagctctgttagagtggcaggagaccggcgtcaccgttaccagcatctctaaggacttcttctccaagttcgaccctaacgctagcgccggcaagaagtactcccccggcagcgacgagttcaactctctcgcccagaacatcgccagcgccgctgaccgattcctgtctaccgtcaactaccaccgaaactctaacggctctctgtctgaggagtacgaccgaaactccggctacatgaccggcgcgagagacctgacctggtctcacgctgctttcattaccgctggcctcgcccgagctggcaccccttctttc

SEQ ID NO: 69 - protein, 20 aa, Apophysomyces trapeziformis
MMMSWKSLLLLLGPIAAATA

SEQ ID NO: 70 - DNA, 60 bp, Apophysomyces trapeziformis
  source 1..60: mol_type = other DNA
atgatgatgtcttggaagtctctgctcctcctgctgggccctatcgccgctgccaccgct

SEQ ID NO: 71 - protein, 443 aa, Apophysomyces trapeziformis
AIPTGNSSLNAWVSSQEDISFSVMLGNINPPGTVKGFVAASLSTAGPDYFYSWVRDSALVSRVVTHKYNTTETGNSTVAGFLQDYVHFQINTQTESTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIIVTQDYQNGVQKPSGLDVSTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSNEYASLAQNIASAADRFLSTVNYHRNPNGSLSEEYDRNTGYMTGARDLTWSHAAFITAGLARAGTPSF

SEQ ID NO: 72 - DNA, 1329 bp, Apophysomyces trapeziformis
  source 1..1329: mol_type = other DNA
gccatccctaccggcaacagcagcctcaacgcttgggttagctctcaggaggacatctctttcagcgtcatgctgggcaacatcaaccctcccggcaccgtcaagggcttcgttgccgctagcctgtctaccgctggccccgactacttctactcttgggtccgagacagcgctctcgtttctcgcgttgttacccacaagtacaacaccaccgagaccggcaacagcaccgttgctggcttcctgcaggactacgttcacttccagattaacacccagaccgagtccaccgtttgcaactgcctgggcgagcctaagttcaaccccgacggcagctcttacaccggcccttggggccgccctcagaacgacggccccgctgagcgcgctagcaccatgattctgatcgctgactccttcctgacccagaccaagaacacctcttacgttgacaacaccctgaagcccgctatttacaaggacctcgactacgtcgttaacacctggtctaacccttgcttcgacctgtgggaggaggttaacggcattcacttctacaccctcatggttatgcgacgaggcctcctggacggcgccaacttcgccacccgaaacggcgacaccagcaaggctagctcttactccagcaccgcttcccagattcagaacaagattgactctttctggagcagctccaagaactacattattgttacccaggactaccagaacggcgtccagaagcctagcggcctcgacgtcagcaccctcattgctgccaacgtcgctggcatgaacgacggcttctacacccccggcagcgacaagatgctcgccaccgccgttgccattgagaacgccatggctaacctgtaccctctgaacaagaacctgccttcttacctgggcaacgctattggccgataccccgaggacacctacaacggcgacggcaactcccagggcaacccttggttcctcgccaccaccgctttcagcgagctgtactaccgagctctgcttgaatggcaggagaccggcgtcaccgttaccagcatttctaaggacttcttctctaagttcgaccctaacgctagcgccggcaagaagtactcccccggctccaacgagtacgctagcctcgcccagaacattgctagcgccgctgaccgattcctgtctaccgttaactaccaccgaaaccctaacggctctctgtctgaggagtacgaccgaaacaccggctacatgaccggcgcccgagacctgacctggtctcacgctgctttcattaccgccggcctggccagggctggcaccccttctttc

SEQ ID NO: 73 - skipped

SEQ ID NO: 74 - DNA, 1329 bp, Apophysomyces trapeziformis
  source 1..1329: mol_type = other DNA
gccatccctaccggcaacagcagcctcaacgcttgggttagctctcaggaggacatctctttcagcgtcatgctgggcaacatcaaccctcccggcaccgtcaagggcttcgttgccgctagcctgtctaccgctggccccgactacttctactcttgggtccgagacagcgctctcgtttctcgcgttgttacccacaagtacaacaccaccgagaccggcaacagcaccgttgctggcttcctgcaggactacgttcacttccagattaacacccagaccgagcccaccgtttgcaactgcctgggcgagcctaagttcaaccccgacggcagctcttacaccggcccttggggccgccctcagaacgacggccccgctgagcgcgctagcaccatgattctgatcgctgactccttcctgacccagaccaagaacacctcttacgttgacaacaccctgaagcccgctatttacaaggacctcgactacgtcgttaacacctggtctaacccttgcttcgacctgtgggaggaggttaacggcattcacttctacaccctcatggttatgcgacgaggcctcctggacggcgccaacttcgccacccgaaacggcgacaccagcaaggctagctcttactccagcaccgcttcccagattcagaacaagattgactctttctggagcagctccaagaactacattattgttacccaggactaccagaacggcgtccagaagcctagcggcctcgacgtcagcaccctcattgctgccaacgtcgctggcatgaacgacggcttctacacccccggcagcgacaagatgctcgccaccgccgttgccattgagaacgccatggctaacctgtaccctctgaacaagaacctgccttcttacctgggcaacgctattggccgataccccgaggacacctacaacggcgacggcaactcccagggcaacccttggttcctcgccaccaccgctttcagcgagctgtactaccgagctctgcttgaatggcaggagaccggcgtcaccgttaccagcatttctaaggacttcttctctaagttcgaccctaacgctagcgccggcaagaagtactcccccggctccaacgagtacgctagcctcgcccagaacattgctagcgccgctgaccgattcctgtctaccgttaactaccaccgaaaccctaacggctctctgtctgaggagtacgaccgaaacaccggctacatgaccggcgcccgagacctgacctggtctcacgctgctttcattaccgccggcctggccagggctggcaccccttctttc

SEQ ID NO: 75 - protein, 605 aa, synthetic construct
  REGION 1..605: note = Chimeric protein
MKLMNSSMKTCVFFILSYFSLLVSSAAVPTSAAVQVESYKYDGTTFSGRIFVKNIAYSKVVTVIYSDGSDNWNNNNNKISAAYSEAISGSNYEYWTFSAKLSGIKQFYVKYEVSGSTYYDNNGTKNYQVQATSATSTTATATTTTSTSTTTTSTGPTSTASVSFPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 76 - protein, 609 aa, synthetic construct
  REGION 1..609: note = Chimeric protein
MKFSKLFTKPTLFIVSIIISVASSETVPTTANVLVKSYTWDGATLSGQIYIKNLAYAKVVSVIYSDANDNWNNNGNKVAASYSAGIDGTNYEYWTFSGAVSGIKQFYVKYDVSGTSYYDNNGTKNYQVTKTSTTTTAATTTTTATTTTGGTGTTTTTTATATATSTDFPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 77 - protein, 605 aa, synthetic construct
  REGION 1..605: note = Chimeric protein
MKLFGTLKASFVFLIGCIVFAMADTVPTTQVKLQSYTYSGGVLSGTIYVQNIDYTKVVTVIYSDGSGNWNNNGNTISASYSSSISGTNYEYWTFSASVSSIQQFYISYLVDGTTYYDNNGGYGANYAVTATTTTASSTTKTTSGTTKTSTTSTATATSTGTSSFPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 78 - protein, 603 aa, synthetic construct
  REGION 1..603: note = Chimeric protein
MKLNSVWKAFSILLMATMAFAATVPKTQVKLEAYTYSDSIFSGRIFVQNIDYTKIVTVYWSDASNKWDSSKYYTEAAYTHSIPGTNYEYWDFSATIGPSGIKQFYISYQVRGVTYYDNNGGYGVNYDVISSPPTTSDATTIPTTTTSAIPSSTTTIPASTGVPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 79 - protein, 605 aa, synthetic construct
  REGION 1..605: note = Chimeric protein
MRPSLVWKSLLMVVLTAAMGTYAQTVPSVPIKLESYTYSDNVFAGRIFIRNIAYTKVVKVFWSDESGNWNGNGNYVDASYSGSISGTNYEYWEFSTTIGSAGISQSYLRYDVSGSTYYDNNGNANYDIEEDSTPTPTTTTTSTTTITTTTTSGTSTPTSVPTEVPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 80 - protein, 613 aa, synthetic construct
  REGION 1..613: note = Chimeric protein
MKPIHLWHYLFLAMTAAFMGVLADVPTSANIELDYYTYKDKVFSGRIYVKNIAYEKTVAVYWSDASGDWNNNGNNVAASYSESISGTDYEYWDFSTTIGSGGIKQSYLKYTVSGNTYYDNNGSNNYDITETTATTTTTTTTTTTTGSTATTSTTSGPTSTTSTTPGPTPTNVPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 81 - protein, 609 aa, synthetic construct
  REGION 1..609: note = Chimeric protein
MQLNLYWKSLAVLALIQMVMAATVPTTQVQLDYYTYSNNVLSGRIYVQNIAYSKVVKVIYSDASGNWNNNGNTISASYVESISGTNYEYWDFSATIGTAGIKQFYLRYDVSGSTYYDNNGGDNNNYNVVATTSTTSSSTTTTTATTTTATKTTSTTSATATPTSSATFPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 82 - protein, 598 aa, synthetic construct
  REGION 1..598: note = Chimeric protein
MVSFNFLKKPVFIVITCLTVSVCAQSVPTSDPVKVKTFSYDGNSFSGQIYIKNIAYEKTVTVIYSDGTGNWNNNNNKIAAAFSEAISGSNYEYWTFSASVPSIKQFYVKYDVSGKTYYDNNGSKDYNVVTSGPTTTSSGPSKTTTNGPAPTSTNTNFPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 83 - protein, 609 aa, synthetic construct
  REGION 1..609: note = Chimeric protein
MKLFGLPLKPSLFFVVSYLSFLASAASIPSSSEVQLDTYAYDGAKFSGKIYVKNIAYEKTVTVVYADGSDNWNNNGNIISATFSTPISGSNYEYWTFSSSISGIKEFYIKYVVSGKTYYDNNGTKNYQVSTTSSTTTASTTTATRTTTAGTTSTSTTAAPTSTSSGSFPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 84 - protein, 608 aa, synthetic construct
  REGION 1..608: note = Chimeric protein
MKFSNLLKKPLLLIAGILAATVVAETVPTTAAVKVKSFTYDGSTFAGQIYVKNIAYTKTVTVIYSDASNNWNNNGNTIAASYSEGISGTNYEYWTFSAPVSGIKQFYVKYVVSGTTYYDNNNSGNYQVTTTTTTTAPTSTTSGGSSTTTGGSTTTATSVPTGVPSGFPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 85 - protein, 620 aa, synthetic construct
  REGION 1..620: note = Chimeric protein
MRFPNILKYFAFALAYLYCSVHASVLLRRAGEVTLSSYSYSNNVLSGSITVQNIAYQKVVAVYWAQGSNWQSTPISASYSSGPNSSGYETWTFSGTAVGATQLYIKYDVSGTSYYYPGNYVNVQVTSSATTTTTTSRTTTTTIRTTSSGTTTAGTTTTTTLATSTTATSSTAPVDKQSLPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 86 - protein, 789 aa, synthetic construct
  REGION 1..789: note = Chimeric protein
MKFSNLLKKPLLLIAGILAATVVAETVPTTAAVKVKSFTYDGSTFAGQIYVKNIAYTKTVTVIYSDASNNWNNNGNTIAASYSEGISGTNYEYWTFSAPVSGIKQFYVKYVVSGTTYYDNNNSGNYQVTTTTTTTAPTSTTSGGSSTTTGGSTTTATSVPTGVPSGFPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPPASWGETSASSVPGTCAATSAIGTYSSVTVTSWPSIVATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 87 - protein, 790 aa, synthetic construct
  REGION 1..790: note = Chimeric protein
MKFSKLFTKPTLFIVSIIISVASSETVPTTANVLVKSYTWDGATLSGQIYIKNLAYAKVVSVIYSDANDNWNNNGNKVAASYSAGIDGTNYEYWTFSGAVSGIKQFYVKYDVSGTSYYDNNGTKNYQVTKTSTTTTAATTTTTATTTTGGTGTTTTTTATATATSTDFPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPPASWGETSASSVPGTCAATSAIGTYSSVTVTSWPSIVATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 88 - protein, 649 aa, synthetic construct
  REGION 1..649: note = Chimeric protein
MPSWKTLFLLLGPIATAAAAPVDKQSLPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPPASWGETSASSVPGTCAATSAIGTYSSVTVTSWPSIVATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 89 - protein, 627 aa, synthetic construct
  REGION 1..627: note = Chimeric protein
MPSWKTLFLLLGPIATAAAAPVDKQSLPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPPSWANSSASTIPSTCSGASVVGSYSRPTATSFPPSQTPKPGVPSGTPYTPLPCATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 90 - DNA, 1818 bp, synthetic construct
  misc_feature 1..1818: note = Encodes chimeric protein
  source 1..1818: mol_type = other DNA
atgaagctcatgaactctagcatgaagacctgcgttttcttcatcctgtcttacttcagcctcctcgtcagcagcgctgccgttcctacctctgctgctgttcaggttgagtcctacaagtacgacggcaccaccttcagcggccgaattttcgtcaagaacattgcttactccaaggttgttaccgtcatatatagcgacggctctgacaactggaacaacaacaacaacaagatcagcgctgcttacagcgaggctattagcggctccaactacgagtactggaccttctctgctaagctgtccggcattaagcagttctacgtcaagtacgaggtttctggctctacctactacgacaacaacggcaccaagaactaccaggtccaggctacctctgccaccagcaccaccgccaccgccaccaccaccacctctaccagcaccaccaccaccagcaccggccctaccagcaccgcaagtgtttctttccctaccggcaacagcaccattagctcttgggtcagcaagcaggaggacatcagcttcagcgagatgctccgaaacgtcaaccctgagggcaccgccaagggcttcgttgccgcgtcgctcagcaccgctggccccgactacttctacacctggacccgagacgccgctctcgtttcccgagttattgcttacaagtacaacaccaccaacgctggcgactctaagatccacggcgttctggacgactacgttaacttccagattaacacccagtctgagcccaccccttgcaactgcctgggcgagcctaagttcaaccctgacggcagctctttcaccggcccttggggccgcccccagaacgacggccccgctgagcgagcctccagtttcatgctgattgccgactctttcctcagccagaccaagaacgcctcttacttcaccaacaccctcaagcctgccatttacaaggacctcgactacgtcgttgacacctggtctaacccttgcttcgacctgtgggaggaggttaacggcatccacttctacaccctcatggttatgcgacgatctctcctcgacggcgctaacttcgccacccgaaacggcgacaactccaaggcctcaacctactccggcgtcgccgccaagattcaggctcgactgaactctttctgggacgccggcaagaactacattaccgttacccaggactacaagaacggcgttgagaagcctagcggcctcgacgttagcaccctcattgctgctaacgtcgctggcatgggcgacggcttctacacccccggcagcgagcgcatcctcgctaccgctctggctttcgagaagagcatggcaagtctgtaccctctgaacaacaacctgcctagccacctgggcaacgccattggccgataccccgaggacacctacaacggcaacggcaactcccagggcaacccttggttcctcgctaccaccgctttcaccgagctgtactaccgcgccattctggagtggaagaacaccggcgttaccgtcacccctatttctaaggacttcttcgtccgattcgacagcagcgctgctcctggcaagaagtacaaccccggctcccaggagttcgctaccctgacccagtctattgctgccgccgctgaccgattcatgtccaccgtccagtaccaccagaaccctaacggctctctgtctgaggagttcgaccgatctagcggctacatgaccggcgcccgagacctgacctggagtcacgctgctttcattaccgctgctcaggcccgagctggctctccttctttctaa

SEQ ID NO: 91 - DNA, 1827 bp, synthetic construct
  misc_feature 1..1827: note = Encodes chimeric protein
  source 1..1827: mol_type = other DNA
atgaagttcagcaagctgttcaccaagcctaccctgttcatcgtttctattattatcagcgttgcttctagcgagaccgtccctaccaccgctaacgtcctcgttaagtcttacacctgggacggcgctaccctgtctggccagatttacattaagaacctcgcttacgccaaggttgttagcgttatttactctgacgctaacgacaactggaacaacaacggcaacaaggttgccgcttcttactctgctggcattgacggcaccaactacgagtactggaccttcagcggcgccgtttccggcatcaagcagttctacgtcaagtacgacgtcagcggcacctcttactacgacaacaacggcaccaagaactaccaggttaccaagacctctaccaccaccaccgccgctaccaccaccaccaccgctaccaccaccaccggcggcaccggcaccaccaccaccaccaccgccaccgccaccgctaccagcaccgacttccctaccggcaacagcaccattagctcttgggtcagcaagcaggaggacatcagcttcagcgagatgctccgaaacgtcaaccctgagggcaccgccaagggcttcgttgccgcgtcgctcagcaccgctggccccgactacttctacacctggacccgagacgccgctctcgtttcccgagttattgcttacaagtacaacaccaccaacgctggcgactctaagatccacggcgttctggacgactacgttaacttccagattaacacccagtctgagcccaccccttgcaactgcctgggcgagcctaagttcaaccctgacggcagctctttcaccggcccttggggccgcccccagaacgacggccccgctgagcgagcctccagtttcatgctgattgccgactctttcctcagccagaccaagaacgcctcttacttcaccaacaccctcaagcctgccatttacaaggacctcgactacgtcgttgacacctggtctaacccttgcttcgacctgtgggaggaggttaacggcatccacttctacaccctcatggttatgcgacgatctctcctcgacggcgctaacttcgccacccgaaacggcgacaactccaaggcctcaacctactccggcgtcgccgccaagattcaggctcgactgaactctttctgggacgccggcaagaactacattaccgttacccaggactacaagaacggcgttgagaagcctagcggcctcgacgttagcaccctcattgctgctaacgtcgctggcatgggcgacggcttctacacccccggcagcgagcgcatcctcgctaccgctctggctttcgagaagagcatggcaagtctgtaccctctgaacaacaacctgcctagccacctgggcaacgccattggccgataccccgaggacacctacaacggcaacggcaactcccagggcaacccttggttcctcgctaccaccgctttcaccgagctgtactaccgcgccattctggagtggaagaacaccggcgttaccgtcacccctatttctaaggacttcttcgtccgattcgacagcagcgctgctcctggcaagaagtacaaccccggctcccaggagttcgctaccctgacccagtctattgctgccgccgctgaccgattcatgtccaccgtccagtaccaccagaaccctaacggctctctgtctgaggagttcgaccgatctagcggctacatgaccggcgcccgagacctgacctggagtcacgctgctttcattaccgctgctcaggcccgagctggctctccttctttc

SEQ ID NO: 92 - DNA, 1815 bp, synthetic construct
  misc_feature 1..1815: note = Encodes chimeric protein
  source 1..1815: mol_type = other DNA
atgaagctgttcggcaccctgaaggcttctttcgttttcctgattggctgcattgttttcgctatggccgacaccgtccctaccacccaggttaagctgcagtcttacacctactctggcggcgtcctgtctggcaccatctacgtccagaacattgactacaccaaggttgttaccgttatatattccgacggctctggcaactggaacaacaacggcaacaccattagcgcttcttacagcagctccatttctggcaccaactacgagtactggaccttctctgccagcgtcagctctattcagcagttctacatctcttacctcgttgacggcaccacctactacgacaacaacggcggctacggcgctaactacgccgttaccgccaccaccaccaccgcttcttccaccaccaagaccacctccggcaccaccaagacctccaccaccagcaccgctaccgccaccagcaccggcacctcttccttccctaccggcaacagcaccattagctcttgggtcagcaagcaggaggacatcagcttcagcgagatgctccgaaacgtcaaccctgagggcaccgccaagggcttcgttgccgcgtcgctcagcaccgctggccccgactacttctacacctggacccgagacgccgctctcgtttcccgagttattgcttacaagtacaacaccaccaacgctggcgactctaagatccacggcgttctggacgactacgttaacttccagattaacacccagtctgagcccaccccttgcaactgcctgggcgagcctaagttcaaccctgacggcagctctttcaccggcccttggggccgcccccagaacgacggccccgctgagcgagcctccagtttcatgctgattgccgactctttcctcagccagaccaagaacgcctcttacttcaccaacaccctcaagcctgccatttacaaggacctcgactacgtcgttgacacctggtctaacccttgcttcgacctgtgggaggaggttaacggcatccacttctacaccctcatggttatgcgacgatctctcctcgacggcgctaacttcgccacccgaaacggcgacaactccaaggcctcaacctactccggcgtcgccgccaagattcaggctcgactgaactctttctgggacgccggcaagaactacattaccgttacccaggactacaagaacggcgttgagaagcctagcggcctcgacgttagcaccctcattgctgctaacgtcgctggcatgggcgacggcttctacacccccggcagcgagcgcatcctcgctaccgctctggctttcgagaagagcatggcaagtctgtaccctctgaacaacaacctgcctagccacctgggcaacgccattggccgataccccgaggacacctacaacggcaacggcaactcccagggcaacccttggttcctcgctaccaccgctttcaccgagctgtactaccgcgccattctggagtggaagaacaccggcgttaccgtcacccctatttctaaggacttcttcgtccgattcgacagcagcgctgctcctggcaagaagtacaaccccggctcccaggagttcgctaccctgacccagtctattgctgccgccgctgaccgattcatgtccaccgtccagtaccaccagaaccctaacggctctctgtctgaggagttcgaccgatctagcggctacatgaccggcgcccgagacctgacctggagtcacgctgctttcattaccgctgctcaggcccgagctggctctccttctttc

SEQ ID NO: 93 - DNA, 1809 bp, synthetic construct
  misc_feature 1..1809: note = Encodes chimeric protein
  source 1..1809: mol_type = other DNA
atgaagctgaactccgtttggaaggctttcagcattctgctcatggctactatggctttcgctgctaccgtccccaagacccaggttaagttagaggcttacacctactctgactctattttctctggccgaattttcgtccagaacatcgactacaccaagattgttaccgtttactggtctgacgcctctaacaagtgggacagctctaagtactacaccgaggccgcttacacccacagcatccccggcaccaactacgagtactgggacttcagcgctaccattggccctagcggcatcaagcagttctacatctcttaccaggtccgaggcgtcacctactacgacaacaacggcggctacggcgtcaactacgacgtcatctccagccctcctaccacctctgacgccaccaccattcctaccaccaccacctccgctattcctagctctaccaccaccatccctgccagcaccggcgtccctaccggcaacagcaccattagctcttgggtcagcaagcaggaggacatcagcttcagcgagatgctccgaaacgtcaaccctgagggcaccgccaagggcttcgttgccgcgtcgctcagcaccgctggccccgactacttctacacctggacccgagacgccgctctcgtttcccgagttattgcttacaagtacaacaccaccaacgctggcgactctaagatccacggcgttctggacgactacgttaacttccagattaacacccagtctgagcccaccccttgcaactgcctgggcgagcctaagttcaaccctgacggcagctctttcaccggcccttggggccgcccccagaacgacggccccgctgagcgagcctccagtttcatgctgattgccgactctttcctcagccagaccaagaacgcctcttacttcaccaacaccctcaagcctgccatttacaaggacctcgactacgtcgttgacacctggtctaacccttgcttcgacctgtgggaggaggttaacggcatccacttctacaccctcatggttatgcgacgatctctcctcgacggcgctaacttcgccacccgaaacggcgacaactccaaggcctcaacctactccggcgtcgccgccaagattcaggctcgactgaactctttctgggacgccggcaagaactacattaccgttacccaggactacaagaacggcgttgagaagcctagcggcctcgacgttagcaccctcattgctgctaacgtcgctggcatgggcgacggcttctacacccccggcagcgagcgcatcctcgctaccgctctggctttcgagaagagcatggcaagtctgtaccctctgaacaacaacctgcctagccacctgggcaacgccattggccgataccccgaggacacctacaacggcaacggcaactcccagggcaacccttggttcctcgctaccaccgctttcaccgagctgtactaccgcgccattctggagtggaagaacaccggcgttaccgtcacccctatttctaaggacttcttcgtccgattcgacagcagcgctgctcctggcaagaagtacaaccccggctcccaggagttcgctaccctgacccagtctattgctgccgccgctgaccgattcatgtccaccgtccagtaccaccagaaccctaacggctctctgtctgaggagttcgaccgatctagcggctacatgaccggcgcccgagacctgacctggagtcacgctgctttcattaccgctgctcaggcccgagctggctctccttctttc

SEQ ID NO: 94 - DNA, 1815 bp, synthetic construct
  misc_feature 1..1815: note = Encodes chimeric protein
  source 1..1815: mol_type = other DNA
atgcgccctagcctcgtttggaagtctctcctgatggttgtcctgaccgctgcaatgggcacctacgcccagaccgtccctagcgttcctattaagttagaatcttacacctactctgacaacgttttcgctggccgaattttcatccgaaacattgcttacaccaaggttgtcaaggttttctggtctgacgagtccggcaactggaacggcaacggcaactacgttgacgcctcttactctggctccatctccggcaccaactacgagtactgggagttcagcaccaccattggctctgccggcatctcccagtcttacctgcgatacgacgtcagcggctctacctactacgacaacaacggcaacgctaactacgacattgaggaggacagcacccctacccctaccaccaccaccacctctaccaccaccatcaccaccaccaccacctccggcacctctacccctacctctgttcctaccgaggtccctaccggcaacagcaccattagctcttgggtcagcaagcaggaggacatcagcttcagcgagatgctccgaaacgtcaaccctgagggcaccgccaagggcttcgttgccgcgtcgctcagcaccgctggccccgactacttctacacctggacccgagacgccgctctcgtttcccgagttattgcttacaagtacaacaccaccaacgctggcgactctaagatccacggcgttctggacgactacgttaacttccagattaacacccagtctgagcccaccccttgcaactgcctgggcgagcctaagttcaaccctgacggcagctctttcaccggcccttggggccgcccccagaacgacggccccgctgagcgagcctccagtttcatgctgattgccgactctttcctcagccagaccaagaacgcctcttacttcaccaacaccctcaagcctgccatttacaaggacctcgactacgtcgttgacacctggtctaacccttgcttcgacctgtgggaggaggttaacggcatccacttctacaccctcatggttatgcgacgatctctcctcgacggcgctaacttcgccacccgaaacggcgacaactccaaggcctcaacctactccggcgtcgccgccaagattcaggctcgactgaactctttctgggacgccggcaagaactacattaccgttacccaggactacaagaacggcgttgagaagcctagcggcctcgacgttagcaccctcattgctgctaacgtcgctggcatgggcgacggcttctacacccccggcagcgagcgcatcctcgctaccgctctggctttcgagaagagcatggcaagtctgtaccctctgaacaacaacctgcctagccacctgggcaacgccattggccgataccccgaggacacctacaacggcaacggcaactcccagggcaacccttggttcctcgctaccaccgctttcaccgagctgtactaccgcgccattctggagtggaagaacaccggcgttaccgtcacccctatttctaaggacttcttcgtccgattcgacagcagcgctgctcctggcaagaagtacaaccccggctcccaggagttcgctaccctgacccagtctattgctgccgccgctgaccgattcatgtccaccgtccagtaccaccagaaccctaacggctctctgtctgaggagttcgaccgatctagcggctacatgaccggcgcccgagacctgacctggagtcacgctgctttcattaccgctgctcaggcccgagctggctctccttctttc

SEQ ID NO: 95 - DNA, 1839 bp, synthetic construct
  misc_feature 1..1839: note = Encodes chimeric protein
  source 1..1839: mol_type = other DNA
atgaagcctattcacctgtggcactacctgttcctcgccatgaccgctgccttcatgggcgtcctcgctgacgtccctacctccgccaacattgaactcgactactacacctacaaggacaaggttttctccggccgaatttacgtcaagaacatcgcttacgaaaaaaccgttgctgtttactggtctgacgctagtggcgactggaacaacaacggcaacaacgttgccgcttcttactctgagtccatctccggcaccgactacgagtactgggacttcagcaccaccattggctctggcggcatcaagcagtcttacctcaagtacaccgttagcggcaacacctactacgacaacaacggctctaacaactacgacattaccgagaccaccgctaccaccaccaccaccaccaccaccaccaccaccaccggctctaccgccaccacctctaccacctctggccctaccagcaccacctctaccacccccggccctacccctaccaacgttcctaccggcaacagcaccattagctcttgggtcagcaagcaggaggacatcagcttcagcgagatgctccgaaacgtcaaccctgagggcaccgccaagggcttcgttgccgcgtcgctcagcaccgctggccccgactacttctacacctggacccgagacgccgctctcgtttcccgagttattgcttacaagtacaacaccaccaacgctggcgactctaagatccacggcgttctggacgactacgttaacttccagattaacacccagtctgagcccaccccttgcaactgcctgggcgagcctaagttcaaccctgacggcagctctttcaccggcccttggggccgcccccagaacgacggccccgctgagcgagcctccagtttcatgctgattgccgactctttcctcagccagaccaagaacgcctcttacttcaccaacaccctcaagcctgccatttacaaggacctcgactacgtcgttgacacctggtctaacccttgcttcgacctgtgggaggaggttaacggcatccacttctacaccctcatggttatgcgacgatctctcctcgacggcgctaacttcgccacccgaaacggcgacaactccaaggcctcaacctactccggcgtcgccgccaagattcaggctcgactgaactctttctgggacgccggcaagaactacattaccgttacccaggactacaagaacggcgttgagaagcctagcggcctcgacgttagcaccctcattgctgctaacgtcgctggcatgggcgacggcttctacacccccggcagcgagcgcatcctcgctaccgctctggctttcgagaagagcatggcaagtctgtaccctctgaacaacaacctgcctagccacctgggcaacgccattggccgataccccgaggacacctacaacggcaacggcaactcccagggcaacccttggttcctcgctaccaccgctttcaccgagctgtactaccgcgccattctggagtggaagaacaccggcgttaccgtcacccctatttctaaggacttcttcgtccgattcgacagcagcgctgctcctggcaagaagtacaaccccggctcccaggagttcgctaccctgacccagtctattgctgccgccgctgaccgattcatgtccaccgtccagtaccaccagaaccctaacggctctctgtctgaggagttcgaccgatctagcggctacatgaccggcgcccgagacctgacctggagtcacgctgctttcattaccgctgctcaggcccgagctggctctccttctttc

SEQ ID NO: 96 - DNA, 1827 bp, synthetic construct
  misc_feature 1..1827: note = Encodes chimeric protein
  source 1..1827: mol_type = other DNA
atgcagctgaacctgtactggaagagcctcgccgtcctcgctctgattcagatggttatggctgctaccgtccctaccacccaggtccagctcgactactacacctactctaacaacgtcctgtctggccgaatctacgttcagaacattgcttacagcaaggttgttaaggttatctatagcgacgcctccggcaactggaacaacaacggcaacaccatctccgcttcttacgtcgagtctatttccggcaccaactacgagtactgggacttcagcgccaccattggcaccgctggcatcaagcagttctacctgcgatacgacgtttccggctccacctactacgacaacaacggcggcgacaacaacaactacaacgttgttgctaccacctctaccacctctagctctaccaccaccaccaccgctaccaccaccaccgccaccaagaccaccagcaccacctctgccaccgctacccctacctctagcgccaccttccctaccggcaacagcaccattagctcttgggtcagcaagcaggaggacatcagcttcagcgagatgctccgaaacgtcaaccctgagggcaccgccaagggcttcgttgccgcgtcgctcagcaccgctggccccgactacttctacacctggacccgagacgccgctctcgtttcccgagttattgcttacaagtacaacaccaccaacgctggcgactctaagatccacggcgttctggacgactacgttaacttccagattaacacccagtctgagcccaccccttgcaactgcctgggcgagcctaagttcaaccctgacggcagctctttcaccggcccttggggccgcccccagaacgacggccccgctgagcgagcctccagtttcatgctgattgccgactctttcctcagccagaccaagaacgcctcttacttcaccaacaccctcaagcctgccatttacaaggacctcgactacgtcgttgacacctggtctaacccttgcttcgacctgtgggaggaggttaacggcatccacttctacaccctcatggttatgcgacgatctctcctcgacggcgctaacttcgccacccgaaacggcgacaactccaaggcctcaacctactccggcgtcgccgccaagattcaggctcgactgaactctttctgggacgccggcaagaactacattaccgttacccaggactacaagaacggcgttgagaagcctagcggcctcgacgttagcaccctcattgctgctaacgtcgctggcatgggcgacggcttctacacccccggcagcgagcgcatcctcgctaccgctctggctttcgagaagagcatggcaagtctgtaccctctgaacaacaacctgcctagccacctgggcaacgccattggccgataccccgaggacacctacaacggcaacggcaactcccagggcaacccttggttcctcgctaccaccgctttcaccgagctgtactaccgcgccattctggagtggaagaacaccggcgttaccgtcacccctatttctaaggacttcttcgtccgattcgacagcagcgctgctcctggcaagaagtacaaccccggctcccaggagttcgctaccctgacccagtctattgctgccgccgctgaccgattcatgtccaccgtccagtaccaccagaaccctaacggctctctgtctgaggagttcgaccgatctagcggctacatgaccggcgcccgagacctgacctggagtcacgctgctttcattaccgctgctcaggcccgagctggctctccttctttc

SEQ ID NO: 97 - DNA, 1794 bp, synthetic construct
  misc_feature 1..1794: note = Encodes chimeric protein
  source 1..1794: mol_type = other DNA
atggtttctttcaacttcctgaagaagcctgttttcattgttatcacctgcctcaccgtcagcgtttgcgcccagtctgttcctaccagcgaccccgttaaggttaagaccttctcttacgacggcaactctttctctggccagatttacatcaagaacattgcttacgagaagaccgttaccgttatttactccgacggcaccggcaactggaacaacaacaacaacaagattgccgctgctttcagcgaggccatctccggctctaactacgagtactggaccttcagcgctagcgttcctagcatcaagcagttctacgtcaagtacgacgtttccggcaagacctactacgacaacaacggctctaaggactacaacgtcgttaccagcggccctaccaccaccagctctggccctagcaagaccaccaccaacggccccgctcctaccagcaccaacaccaacttccctaccggcaacagcaccattagctcttgggtcagcaagcaggaggacatcagcttcagcgagatgctccgaaacgtcaaccctgagggcaccgccaagggcttcgttgccgcgtcgctcagcaccgctggccccgactacttctacacctggacccgagacgccgctctcgtttcccgagttattgcttacaagtacaacaccaccaacgctggcgactctaagatccacggcgttctggacgactacgttaacttccagattaacacccagtctgagcccaccccttgcaactgcctgggcgagcctaagttcaaccctgacggcagctctttcaccggcccttggggccgcccccagaacgacggccccgctgagcgagcctccagtttcatgctgattgccgactctttcctcagccagaccaagaacgcctcttacttcaccaacaccctcaagcctgccatttacaaggacctcgactacgtcgttgacacctggtctaacccttgcttcgacctgtgggaggaggttaacggcatccacttctacaccctcatggttatgcgacgatctctcctcgacggcgctaacttcgccacccgaaacggcgacaactccaaggcctcaacctactccggcgtcgccgccaagattcaggctcgactgaactctttctgggacgccggcaagaactacattaccgttacccaggactacaagaacggcgttgagaagcctagcggcctcgacgttagcaccctcattgctgctaacgtcgctggcatgggcgacggcttctacacccccggcagcgagcgcatcctcgctaccgctctggctttcgagaagagcatggcaagtctgtaccctctgaacaacaacctgcctagccacctgggcaacgccattggccgataccccgaggacacctacaacggcaacggcaactcccagggcaacccttggttcctcgctaccaccgctttcaccgagctgtactaccgcgccattctggagtggaagaacaccggcgttaccgtcacccctatttctaaggacttcttcgtccgattcgacagcagcgctgctcctggcaagaagtacaaccccggctcccaggagttcgctaccctgacccagtctattgctgccgccgctgaccgattcatgtccaccgtccagtaccaccagaaccctaacggctctctgtctgaggagttcgaccgatctagcggctacatgaccggcgcccgagacctgacctggagtcacgctgctttcattaccgctgctcaggcccgagctggctctccttctttc

SEQ ID NO: 98 - DNA, 1827 bp, synthetic construct
  misc_feature 1..1827: note = Encodes chimeric polypeptide
  source 1..1827: mol_type = other DNA
atgaagctgttcggcctgcccctcaagcctagcctgttcttcgtcgtctcttacctgtctttcctcgcttctgctgccagcatccctagcagcagcgaggtccagctcgacacctacgcttacgacggcgccaagttctctggcaagatttacgtcaagaacattgcttacgagaagaccgttaccgttgtttacgctgacggctctgacaactggaacaacaacggcaacattattagcgctaccttcagcacccctatttccggctctaactacgagtactggaccttcagctctagcattagcggcattaaggagttctacatcaagtacgtcgtttccggcaagacctactacgacaacaacggcaccaagaactaccaggtttccaccaccagctctaccaccaccgctagcaccaccaccgctacccgaaccaccaccgctggcaccaccagcacctctaccaccgctgcccctacctctacctccagcggctccttccctaccggcaacagcaccattagctcttgggtcagcaagcaggaggacatcagcttcagcgagatgctccgaaacgtcaaccctgagggcaccgccaagggcttcgttgccgcgtcgctcagcaccgctggccccgactacttctacacctggacccgagacgccgctctcgtttcccgagttattgcttacaagtacaacaccaccaacgctggcgactctaagatccacggcgttctggacgactacgttaacttccagattaacacccagtctgagcccaccccttgcaactgcctgggcgagcctaagttcaaccctgacggcagctctttcaccggcccttggggccgcccccagaacgacggccccgctgagcgagcctccagtttcatgctgattgccgactctttcctcagccagaccaagaacgcctcttacttcaccaacaccctcaagcctgccatttacaaggacctcgactacgtcgttgacacctggtctaacccttgcttcgacctgtgggaggaggttaacggcatccacttctacaccctcatggttatgcgacgatctctcctcgacggcgctaacttcgccacccgaaacggcgacaactccaaggcctcaacctactccggcgtcgccgccaagattcaggctcgactgaactctttctgggacgccggcaagaactacattaccgttacccaggactacaagaacggcgttgagaagcctagcggcctcgacgttagcaccctcattgctgctaacgtcgctggcatgggcgacggcttctacacccccggcagcgagcgcatcctcgctaccgctctggctttcgagaagagcatggcaagtctgtaccctctgaacaacaacctgcctagccacctgggcaacgccattggccgataccccgaggacacctacaacggcaacggcaactcccagggcaacccttggttcctcgctaccaccgctttcaccgagctgtactaccgcgccattctggagtggaagaacaccggcgttaccgtcacccctatttctaaggacttcttcgtccgattcgacagcagcgctgctcctggcaagaagtacaaccccggctcccaggagttcgctaccctgacccagtctattgctgccgccgctgaccgattcatgtccaccgtccagtaccaccagaaccctaacggctctctgtctgaggagttcgaccgatctagcggctacatgaccggcgcccgagacctgacctggagtcacgctgctttcattaccgctgctcaggcccgagctggctctccttctttc

SEQ ID NO: 99 - DNA, 1824 bp, synthetic construct
  misc_feature 1..1824: note = Encodes chimeric protein
  source 1..1824: mol_type = other DNA
atgaagttctctaacctcctgaagaagcccctcctgctcattgctggcattctcgctgccaccgtcgttgccgagaccgtccctaccaccgctgctgttaaggttaagtctttcacctacgacggcagcaccttcgctggccagatttacgtcaagaacatcgcttacaccaagaccgttaccgttatttactctgacgcctctaacaactggaacaacaacggcaacaccattgccgcctcttacagcgagggcatctctggcaccaactacgagtactggaccttcagcgcccctgtttccggcatcaagcagttctacgtcaagtacgttgtcagcggcaccacctactacgacaacaacaactctggcaactaccaggttaccaccaccaccaccaccaccgcccctaccagcaccacctccggcggctctagcaccaccaccggcggctccaccaccaccgccacctctgtccctaccggcgtccctagcggcttccctaccggcaacagcaccattagctcttgggtcagcaagcaggaggacatcagcttcagcgagatgctccgaaacgtcaaccctgagggcaccgccaagggcttcgttgccgcgtcgctcagcaccgctggccccgactacttctacacctggacccgagacgccgctctcgtttcccgagttattgcttacaagtacaacaccaccaacgctggcgactctaagatccacggcgttctggacgactacgttaacttccagattaacacccagtctgagcccaccccttgcaactgcctgggcgagcctaagttcaaccctgacggcagctctttcaccggcccttggggccgcccccagaacgacggccccgctgagcgagcctccagtttcatgctgattgccgactctttcctcagccagaccaagaacgcctcttacttcaccaacaccctcaagcctgccatttacaaggacctcgactacgtcgttgacacctggtctaacccttgcttcgacctgtgggaggaggttaacggcatccacttctacaccctcatggttatgcgacgatctctcctcgacggcgctaacttcgccacccgaaacggcgacaactccaaggcctcaacctactccggcgtcgccgccaagattcaggctcgactgaactctttctgggacgccggcaagaactacattaccgttacccaggactacaagaacggcgttgagaagcctagcggcctcgacgttagcaccctcattgctgctaacgtcgctggcatgggcgacggcttctacacccccggcagcgagcgcatcctcgctaccgctctggctttcgagaagagcatggcaagtctgtaccctctgaacaacaacctgcctagccacctgggcaacgccattggccgataccccgaggacacctacaacggcaacggcaactcccagggcaacccttggttcctcgctaccaccgctttcaccgagctgtactaccgcgccattctggagtggaagaacaccggcgttaccgtcacccctatttctaaggacttcttcgtccgattcgacagcagcgctgctcctggcaagaagtacaaccccggctcccaggagttcgctaccctgacccagtctattgctgccgccgctgaccgattcatgtccaccgtccagtaccaccagaaccctaacggctctctgtctgaggagttcgaccgatctagcggctacatgaccggcgcccgagacctgacctggagtcacgctgctttcattaccgctgctcaggcccgagctggctctccttctttc

SEQ ID NO: 100 - DNA, 1860 bp, synthetic construct
  misc_feature 1..1860: note = Encodes chimeric protein
  source 1..1860: mol_type = other DNA
atgcgattccctaacatcctgaagtacttcgctttcgccctcgcttacctgtactgcagcgttcacgcttccgtcctgctgcgacgcgccggcgaggttaccctgagctcttactcctacagcaacaacgtcctgtctggctccattaccgtccagaacattgcctaccagaaggtcgttgctgtttactgggctcagggctctaactggcagtctacccctatttctgcttcttacagcagcggccccaactcttccggctacgagacctggaccttctccggcaccgccgttggcgctacccagctgtacatcaagtacgacgtttctggcacctcttactactaccccggcaactacgtcaacgttcaggttacctctagcgctaccaccaccaccaccacctcccgaaccaccaccaccaccattcgaaccacctctagcggcaccaccaccgccggcaccaccaccaccaccaccctcgctacctctaccaccgctacctccagcaccgctcccgttgacaagcagtctctgcctaccggcaacagcaccattagctcttgggtcagcaagcaggaggacatcagcttcagcgagatgctccgaaacgtcaaccctgagggcaccgccaagggcttcgttgccgcgtcgctcagcaccgctggccccgactacttctacacctggacccgagacgccgctctcgtttcccgagttattgcttacaagtacaacaccaccaacgctggcgactctaagatccacggcgttctggacgactacgttaacttccagattaacacccagtctgagcccaccccttgcaactgcctgggcgagcctaagttcaaccctgacggcagctctttcaccggcccttggggccgcccccagaacgacggccccgctgagcgagcctccagtttcatgctgattgccgactctttcctcagccagaccaagaacgcctcttacttcaccaacaccctcaagcctgccatttacaaggacctcgactacgtcgttgacacctggtctaacccttgcttcgacctgtgggaggaggttaacggcatccacttctacaccctcatggttatgcgacgatctctcctcgacggcgctaacttcgccacccgaaacggcgacaactccaaggcctcaacctactccggcgtcgccgccaagattcaggctcgactgaactctttctgggacgccggcaagaactacattaccgttacccaggactacaagaacggcgttgagaagcctagcggcctcgacgttagcaccctcattgctgctaacgtcgctggcatgggcgacggcttctacacccccggcagcgagcgcatcctcgctaccgctctggctttcgagaagagcatggcaagtctgtaccctctgaacaacaacctgcctagccacctgggcaacgccattggccgataccccgaggacacctacaacggcaacggcaactcccagggcaacccttggttcctcgctaccaccgctttcaccgagctgtactaccgcgccattctggagtggaagaacaccggcgttaccgtcacccctatttctaaggacttcttcgtccgattcgacagcagcgctgctcctggcaagaagtacaaccccggctcccaggagttcgctaccctgacccagtctattgctgccgccgctgaccgattcatgtccaccgtccagtaccaccagaaccctaacggctctctgtctgaggagttcgaccgatctagcggctacatgaccggcgcccgagacctgacctggagtcacgctgctttcattaccgctgctcaggcccgagctggctctccttctttc

SEQ ID NO: 101 - DNA, 2367 bp, synthetic construct
  misc_feature 1..2367: note = Encodes chimeric protein
  source 1..2367: mol_type = other DNA
atgaagttctctaacctcctgaagaagcccctcctgctcattgctggcattctcgctgccaccgtcgttgccgagaccgtccctaccaccgctgctgttaaggttaagtctttcacctacgacggcagcaccttcgctggccagatttacgtcaagaacatcgcttacaccaagaccgttaccgttatttactctgacgcctctaacaactggaacaacaacggcaacaccattgccgcctcttacagcgagggcatctctggcaccaactacgagtactggaccttcagcgcccctgtttccggcatcaagcagttctacgtcaagtacgttgtcagcggcaccacctactacgacaacaacaactctggcaactaccaggttaccaccaccaccaccaccaccgcccctaccagcaccacctccggcggctctagcaccaccaccggcggctccaccaccaccgccacctctgtccctaccggcgtccctagcggcttccctaccggcaacagcaccattagctcttgggtcagcaagcaggaggacatcagcttcagcgagatgctccgaaacgtcaaccctgagggcaccgccaagggcttcgttgccgcgtcgctcagcaccgctggccccgactacttctacacctggacccgagacgccgctctcgtttcccgagttattgcttacaagtacaacaccaccaacgctggcgactctaagatccacggcgttctggacgactacgttaacttccagattaacacccagtctgagcccaccccttgcaactgcctgggcgagcctaagttcaaccctgacggcagctctttcaccggcccttggggccgcccccagaacgacggccccgctgagcgagcctccagtttcatgctgattgccgactctttcctcagccagaccaagaacgcctcttacttcaccaacaccctcaagcctgccatttacaaggacctcgactacgtcgttgacacctggtctaacccttgcttcgacctgtgggaggaggttaacggcatccacttctacaccctcatggttatgcgacgatctctcctcgacggcgctaacttcgccacccgaaacggcgacaactccaaggcctcaacctactccggcgtcgccgccaagattcaggctcgactgaactctttctgggacgccggcaagaactacattaccgttacccaggactacaagaacggcgttgagaagcctagcggcctcgacgttagcaccctcattgctgctaacgtcgctggcatgggcgacggcttctacacccccggcagcgagcgcatcctcgctaccgctctggctttcgagaagagcatggcaagtctgtaccctctgaacaacaacctgcctagccacctgggcaacgccattggccgataccccgaggacacctacaacggcaacggcaactcccagggcaacccttggttcctcgctaccaccgctttcaccgagctgtactaccgcgccattctggagtggaagaacaccggcgttaccgtcacccctatttctaaggacttcttcgtccgattcgacagcagcgctgctcctggcaagaagtacaaccccggctcccaggagttcgctaccctgacccagtctattgctgccgccgctgaccgattcatgtccaccgtccagtaccaccagaaccctaacggctctctgtctgaggagttcgaccgatctagcggctacatgaccggcgcccgagacctgacctggagtcacgctgctttcattaccgctgctcaggcccgagctggctctcctcctgcttcttggggcgagacctccgcttctagcgtccccggcacctgcgctgctaccagcgctattggcacctactctagcgtcaccgttacctcttggccttccattgttgctaccggcggcaccaccaccaccgccacccctaccggctctggctctgttaccagcaccagcaagaccaccgctaccgcttctaagacctccacctctacctctagcaccagctgcaccacccctaccgccgtcgccgttaccttcgacctgaccgctaccaccacctacggcgagaacatttacctggttggctccattagccagctgggcgactgggagacctccgacggcatcgccctgtctgctgacaagtacaccagctccgaccctctgtggtacgttaccgttaccctgcccgccggcgagtctttcgagtacaagttcatccgaattgagtctgacgactctgttgagtgggagagcgaccccaaccgagagtacaccgttcctcaggcttgcggcaccagcaccgctaccgtcaccgacacctggcgc

SEQ ID NO: 102 - DNA, 2370 bp, synthetic construct
  misc_feature 1..2370: note = Encodes chimeric protein
  source 1..2370: mol_type = other DNA
atgaagttcagcaagctgttcaccaagcctaccctgttcatcgtttctattattatcagcgttgcttctagcgagaccgtccctaccaccgctaacgtcctcgttaagtcttacacctgggacggcgctaccctgtctggccagatttacattaagaacctcgcttacgccaaggttgttagcgttatttactctgacgctaacgacaactggaacaacaacggcaacaaggttgccgcttcttactctgctggcattgacggcaccaactacgagtactggaccttcagcggcgccgtttccggcatcaagcagttctacgtcaagtacgacgtcagcggcacctcttactacgacaacaacggcaccaagaactaccaggttaccaagacctctaccaccaccaccgccgctaccaccaccaccaccgctaccaccaccaccggcggcaccggcaccaccaccaccaccaccgccaccgccaccgctaccagcaccgacttccctaccggcaacagcaccattagctcttgggtcagcaagcaggaggacatcagcttcagcgagatgctccgaaacgtcaaccctgagggcaccgccaagggcttcgttgccgcgtcgctcagcaccgctggccccgactacttctacacctggacccgagacgccgctctcgtttcccgagttattgcttacaagtacaacaccaccaacgctggcgactctaagatccacggcgttctggacgactacgttaacttccagattaacacccagtctgagcccaccccttgcaactgcctgggcgagcctaagttcaaccctgacggcagctctttcaccggcccttggggccgcccccagaacgacggccccgctgagcgagcctccagtttcatgctgattgccgactctttcctcagccagaccaagaacgcctcttacttcaccaacaccctcaagcctgccatttacaaggacctcgactacgtcgttgacacctggtctaacccttgcttcgacctgtgggaggaggttaacggcatccacttctacaccctcatggttatgcgacgatctctcctcgacggcgctaacttcgccacccgaaacggcgacaactccaaggcctcaacctactccggcgtcgccgccaagattcaggctcgactgaactctttctgggacgccggcaagaactacattaccgttacccaggactacaagaacggcgttgagaagcctagcggcctcgacgttagcaccctcattgctgctaacgtcgctggcatgggcgacggcttctacacccccggcagcgagcgcatcctcgctaccgctctggctttcgagaagagcatggcaagtctgtaccctctgaacaacaacctgcctagccacctgggcaacgccattggccgataccccgaggacacctacaacggcaacggcaactcccagggcaacccttggttcctcgctaccaccgctttcaccgagctgtactaccgcgccattctggagtggaagaacaccggcgttaccgtcacccctatttctaaggacttcttcgtccgattcgacagcagcgctgctcctggcaagaagtacaaccccggctcccaggagttcgctaccctgacccagtctattgctgccgccgctgaccgattcatgtccaccgtccagtaccaccagaaccctaacggctctctgtctgaggagttcgaccgatctagcggctacatgaccggcgcccgagacctgacctggagtcacgctgctttcattaccgctgctcaggcccgagctggctctcctcctgcttcttggggcgagacctccgcttctagcgtccccggcacctgcgctgctaccagcgctattggcacctactctagcgtcaccgttacctcttggccttccattgttgctaccggcggcaccaccaccaccgccacccctaccggctctggctctgttaccagcaccagcaagaccaccgctaccgcttctaagacctccacctctacctctagcaccagctgcaccacccctaccgccgtcgccgttaccttcgacctgaccgctaccaccacctacggcgagaacatttacctggttggctccattagccagctgggcgactgggagacctccgacggcatcgccctgtctgctgacaagtacaccagctccgaccctctgtggtacgttaccgttaccctgcccgccggcgagtctttcgagtacaagttcatccgaattgagtctgacgactctgttgagtgggagagcgaccccaaccgagagtacaccgttcctcaggcttgcggcaccagcaccgctaccgtcaccgacacctggcgc

SEQ ID NO: 103 - DNA, 1947 bp, synthetic construct
  misc_feature 1..1947: note = Encodes chimeric protein
  source 1..1947: mol_type = other DNA
atgccttcttggaaaaccctgttcctcctcctgggccctatcgccaccgctgccgctgctcccgttgacaagcagtctctgcctaccggcaacagcaccattagctcttgggtcagcaagcaggaggacatcagcttcagcgagatgctccgaaacgtcaaccctgagggcaccgccaagggcttcgttgccgcgtcgctcagcaccgctggccccgactacttctacacctggacccgagacgccgctctcgtttcccgagttattgcttacaagtacaacaccaccaacgctggcgactctaagatccacggcgttctggacgactacgttaacttccagattaacacccagtctgagcccaccccttgcaactgcctgggcgagcctaagttcaaccctgacggcagctctttcaccggcccttggggccgcccccagaacgacggccccgctgagcgagcctccagtttcatgctgattgccgactctttcctcagccagaccaagaacgcctcttacttcaccaacaccctcaagcctgccatttacaaggacctcgactacgtcgttgacacctggtctaacccttgcttcgacctgtgggaggaggttaacggcatccacttctacaccctcatggttatgcgacgatctctcctcgacggcgctaacttcgccacccgaaacggcgacaactccaaggcctcaacctactccggcgtcgccgccaagattcaggctcgactgaactctttctgggacgccggcaagaactacattaccgttacccaggactacaagaacggcgttgagaagcctagcggcctcgacgttagcaccctcattgctgctaacgtcgctggcatgggcgacggcttctacacccccggcagcgagcgcatcctcgctaccgctctggctttcgagaagagcatggcaagtctgtaccctctgaacaacaacctgcctagccacctgggcaacgccattggccgataccccgaggacacctacaacggcaacggcaactcccagggcaacccttggttcctcgctaccaccgctttcaccgagctgtactaccgcgccattctggagtggaagaacaccggcgttaccgtcacccctatttctaaggacttcttcgtccgattcgacagcagcgctgctcctggcaagaagtacaaccccggctcccaggagttcgctaccctgacccagtctattgctgccgccgctgaccgattcatgtccaccgtccagtaccaccagaaccctaacggctctctgtctgaggagttcgaccgatctagcggctacatgaccggcgcccgagacctgacctggagtcacgctgctttcattaccgctgctcaggcccgagctggctctcctcctgcttcttggggcgagacctccgcttctagcgtccccggcacctgcgctgctaccagcgctattggcacctactctagcgtcaccgttacctcttggccttccattgttgctaccggcggcaccaccaccaccgccacccctaccggctctggctctgttaccagcaccagcaagaccaccgctaccgcttctaagacctccacctctacctctagcaccagctgcaccacccctaccgccgtcgccgttaccttcgacctgaccgctaccaccacctacggcgagaacatttacctggttggctccattagccagctgggcgactgggagacctccgacggcatcgccctgtctgctgacaagtacaccagctccgaccctctgtggtacgttaccgttaccctgcccgccggcgagtctttcgagtacaagttcatccgaattgagtctgacgactctgttgagtgggagagcgaccccaaccgagagtacaccgttcctcaggcttgcggcaccagcaccgctaccgtcaccgacacctggcgc

SEQ ID NO: 104 - DNA, 1881 bp, synthetic construct
  misc_feature 1..1881: note = Encodes chimeric protein
  source 1..1881: mol_type = other DNA
atgccttcttggaaaaccctgttcctcctcctgggccctatcgccaccgctgccgctgctcccgttgacaagcagtctctgcctaccggcaacagcaccattagctcttgggtcagcaagcaggaggacatcagcttcagcgagatgctccgaaacgtcaaccctgagggcaccgccaagggcttcgttgccgcgtcgctcagcaccgctggccccgactacttctacacctggacccgagacgccgctctcgtttcccgagttattgcttacaagtacaacaccaccaacgctggcgactctaagatccacggcgttctggacgactacgttaacttccagattaacacccagtctgagcccaccccttgcaactgcctgggcgagcctaagttcaaccctgacggcagctctttcaccggcccttggggccgcccccagaacgacggccccgctgagcgagcctccagtttcatgctgattgccgactctttcctcagccagaccaagaacgcctcttacttcaccaacaccctcaagcctgccatttacaaggacctcgactacgtcgttgacacctggtctaacccttgcttcgacctgtgggaggaggttaacggcatccacttctacaccctcatggttatgcgacgatctctcctcgacggcgctaacttcgccacccgaaacggcgacaactccaaggcctcaacctactccggcgtcgccgccaagattcaggctcgactgaactctttctgggacgccggcaagaactacattaccgttacccaggactacaagaacggcgttgagaagcctagcggcctcgacgttagcaccctcattgctgctaacgtcgctggcatgggcgacggcttctacacccccggcagcgagcgcatcctcgctaccgctctggctttcgagaagagcatggcaagtctgtaccctctgaacaacaacctgcctagccacctgggcaacgccattggccgataccccgaggacacctacaacggcaacggcaactcccagggcaacccttggttcctcgctaccaccgctttcaccgagctgtactaccgcgccattctggagtggaagaacaccggcgttaccgtcacccctatttctaaggacttcttcgtccgattcgacagcagcgctgctcctggcaagaagtacaaccccggctcccaggagttcgctaccctgacccagtctattgctgccgccgctgaccgattcatgtccaccgtccagtaccaccagaaccctaacggctctctgtctgaggagttcgaccgatctagcggctacatgaccggcgcccgagacctgacctggagtcacgctgctttcattaccgctgctcaggcccgagctggctctcctccttcttgggctaacagctctgcttctaccattcctagcacctgcagcggcgctagcgtcgttggctcttactcccgacctaccgctacctctttccctccttctcagacccctaagcccggcgttcctagcggcaccccttacacccctctgccttgcgccacccctaccagcgtcgccgttaccttccacgagctcgtttctacccagttcggccagaccgtcaaggttgctggcaacgccgctgccctgggcaactggagcaccagcgccgctgttgctctcgacgctgttaactacgctgacaaccaccctctgtggattggcaccgttaacttagaagccggcgacgttgttgagtacaagtacatcaacgtcggccaggacggctccgttacctgggagtccgaccccaaccacacctacaccgttcctgctgtcgcttgcgtcacccaggttgtcaaggaggacacctggcagtct

SEQ ID NO: 105 - protein, 468 aa, synthetic construct
  REGION 1..468: note = Chimeric protein
MPSWKTLIFLLGPITAALAAPVNKQSFPTGNPDISSWVSKQEGISFSEMLRNVNPPGTAKGFVAASLSTAGPDYFYTWTRDAALVSRVIAYKYNTTNAGDNNIHGALQDYVTFQINTQTEPTPCNCLGEPKFNPDGSSFTGPWGRPQNDGPAERASSMILIADSFLAQTKDTAYVTNTLKPAIYKDLDYVVNTWSNPCFDLWEEVNGVHFYTLMVMRRGLLDGADFATRNGDASKASSYSDAASKIKTRLDSFWVSDKNYITVTQDYKEGVKKDTGLDVSTLIAANVAGKDDGFYTPGSDKILATAVAFENAMAKLYPLNQNLDSHLGNSIGRYPEDTYNGNGNSQGNPWFLATTAFTELYYRAILEWKDSGVTVTPVSKDFFAKFDSNAVAGKKYSPNSEEFATLTANIAAAADRFMSTVKYHQNPNGSLSEQFDRHSGYMTGARDLTWSHAAFITAAQARAGTPSF

SEQ ID NO: 106 - protein, 608 aa, synthetic construct
  REGION 1..608: note = Chimeric protein
MKFSNLLKKPLLLIAGILAATVVAETVPTTAAVKVKSFTYDGSTFAGQIYVKNIAYTKTVTVIYSDASNNWNNNGNTIAASYSEGISGTNYEYWTFSAPVSGIKQFYVKYVVSGTTYYDNNNSGNYQVTTTTTTTAPTSTTSGGSSTTTGGSTTTATSVPTGVPSGFPTGNPDISSWVSKQEGISFSEMLRNVNPPGTAKGFVAASLSTAGPDYFYTWTRDAALVSRVIAYKYNTTNAGDNNIHGALQDYVTFQINTQTEPTPCNCLGEPKFNPDGSSFTGPWGRPQNDGPAERASSMILIADSFLAQTKDTAYVTNTLKPAIYKDLDYVVNTWSNPCFDLWEEVNGVHFYTLMVMRRGLLDGADFATRNGDASKASSYSDAASKIKTRLDSFWVSDKNYITVTQDYKEGVKKDTGLDVSTLIAANVAGKDDGFYTPGSDKILATAVAFENAMAKLYPLNQNLDSHLGNSIGRYPEDTYNGNGNSQGNPWFLATTAFTELYYRAILEWKDSGVTVTPVSKDFFAKFDSNAVAGKKYSPNSEEFATLTANIAAAADRFMSTVKYHQNPNGSLSEQFDRHSGYMTGARDLTWSHAAFITAAQARAGTPSF

SEQ ID NO: 107 - protein, 609 aa, synthetic construct
  REGION 1..609: note = Chimeric protein
MKFSKLFTKPTLFIVSIIISVASSETVPTTANVLVKSYTWDGATLSGQIYIKNLAYAKVVSVIYSDANDNWNNNGNKVAASYSAGIDGTNYEYWTFSGAVSGIKQFYVKYDVSGTSYYDNNGTKNYQVTKTSTTTTAATTTTTATTTTGGTGTTTTTTATATATSTDFPTGNPDISSWVSKQEGISFSEMLRNVNPPGTAKGFVAASLSTAGPDYFYTWTRDAALVSRVIAYKYNTTNAGDNNIHGALQDYVTFQINTQTEPTPCNCLGEPKFNPDGSSFTGPWGRPQNDGPAERASSMILIADSFLAQTKDTAYVTNTLKPAIYKDLDYVVNTWSNPCFDLWEEVNGVHFYTLMVMRRGLLDGADFATRNGDASKASSYSDAASKIKTRLDSFWVSDKNYITVTQDYKEGVKKDTGLDVSTLIAANVAGKDDGFYTPGSDKILATAVAFENAMAKLYPLNQNLDSHLGNSIGRYPEDTYNGNGNSQGNPWFLATTAFTELYYRAILEWKDSGVTVTPVSKDFFAKFDSNAVAGKKYSPNSEEFATLTANIAAAADRFMSTVKYHQNPNGSLSEQFDRHSGYMTGARDLTWSHAAFITAAQARAGTPSF

SEQ ID NO: 108 - protein, 649 aa, synthetic construct
  REGION 1..649: note = Chimeric protein
MPSWKTLIFLLGPITAALAAPVNKQSFPTGNPDISSWVSKQEGISFSEMLRNVNPPGTAKGFVAASLSTAGPDYFYTWTRDAALVSRVIAYKYNTTNAGDNNIHGALQDYVTFQINTQTEPTPCNCLGEPKFNPDGSSFTGPWGRPQNDGPAERASSMILIADSFLAQTKDTAYVTNTLKPAIYKDLDYVVNTWSNPCFDLWEEVNGVHFYTLMVMRRGLLDGADFATRNGDASKASSYSDAASKIKTRLDSFWVSDKNYITVTQDYKEGVKKDTGLDVSTLIAANVAGKDDGFYTPGSDKILATAVAFENAMAKLYPLNQNLDSHLGNSIGRYPEDTYNGNGNSQGNPWFLATTAFTELYYRAILEWKDSGVTVTPVSKDFFAKFDSNAVAGKKYSPNSEEFATLTANIAAAADRFMSTVKYHQNPNGSLSEQFDRHSGYMTGARDLTWSHAAFITAAQARAGTPPASWGETSASSVPGTCAATSAIGTYSSVTVTSWPSIVATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 109 - protein, 627 aa, synthetic construct
  REGION 1..627: note = Chimeric protein
MPSWKTLIFLLGPITAALAAPVNKQSFPTGNPDISSWVSKQEGISFSEMLRNVNPPGTAKGFVAASLSTAGPDYFYTWTRDAALVSRVIAYKYNTTNAGDNNIHGALQDYVTFQINTQTEPTPCNCLGEPKFNPDGSSFTGPWGRPQNDGPAERASSMILIADSFLAQTKDTAYVTNTLKPAIYKDLDYVVNTWSNPCFDLWEEVNGVHFYTLMVMRRGLLDGADFATRNGDASKASSYSDAASKIKTRLDSFWVSDKNYITVTQDYKEGVKKDTGLDVSTLIAANVAGKDDGFYTPGSDKILATAVAFENAMAKLYPLNQNLDSHLGNSIGRYPEDTYNGNGNSQGNPWFLATTAFTELYYRAILEWKDSGVTVTPVSKDFFAKFDSNAVAGKKYSPNSEEFATLTANIAAAADRFMSTVKYHQNPNGSLSEQFDRHSGYMTGARDLTWSHAAFITAAQARAGTPPSWANSSASTIPSTCSGASVVGSYSRPTATSFPPSQTPKPGVPSGTPYTPLPCATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 110 - protein, 463 aa, synthetic construct
  REGION 1..463: note = Chimeric protein
MMMSWKSLLLLLGPIAAATAAIPTGNSSLNAWVSSQEDISFSVMLGNINPPGTVKGFVAASLSTAGPDYFYSWVRDSALVSRVVTHKYNTTETGNSTVAGFLEDYVHFQINTQTEPTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSNKNYIIVTQDYQNGVQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSNEFASLAQNIASAADRFLSTVNYHRNSNGSLSEEFDRNTGYMTGARDLTWSHAAFITAGLARAGTPSF

SEQ ID NO: 111 - protein, 608 aa, synthetic construct
  REGION 1..608: note = Chimeric protein
MKFSNLLKKPLLLIAGILAATVVAETVPTTAAVKVKSFTYDGSTFAGQIYVKNIAYTKTVTVIYSDASNNWNNNGNTIAASYSEGISGTNYEYWTFSAPVSGIKQFYVKYVVSGTTYYDNNNSGNYQVTTTTTTTAPTSTTSGGSSTTTGGSTTTATSVPTGVPSGFPTGNSSLNAWVSSQEDISFSVMLGNINPPGTVKGFVAASLSTAGPDYFYSWVRDSALVSRVVTHKYNTTETGNSTVAGFLEDYVHFQINTQTEPTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSNKNYIIVTQDYQNGVQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSNEFASLAQNIASAADRFLSTVNYHRNSNGSLSEEFDRNTGYMTGARDLTWSHAAFITAGLARAGTPSF

SEQ ID NO: 112 - protein, 609 aa, synthetic construct
  REGION 1..609: note = Chimeric protein
MKFSKLFTKPTLFIVSIIISVASSETVPTTANVLVKSYTWDGATLSGQIYIKNLAYAKVVSVIYSDANDNWNNNGNKVAASYSAGIDGTNYEYWTFSGAVSGIKQFYVKYDVSGTSYYDNNGTKNYQVTKTSTTTTAATTTTTATTTTGGTGTTTTTTATATATSTDFPTGNSSLNAWVSSQEDISFSVMLGNINPPGTVKGFVAASLSTAGPDYFYSWVRDSALVSRVVTHKYNTTETGNSTVAGFLEDYVHFQINTQTEPTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSNKNYIIVTQDYQNGVQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSNEFASLAQNIASAADRFLSTVNYHRNSNGSLSEEFDRNTGYMTGARDLTWSHAAFITAGLARAGTPSF

SEQ ID NO: 113 - protein, 644 aa, synthetic construct
  REGION 1..644: note = Chimeric protein
MMMSWKSLLLLLGPIAAATAAIPTGNSSLNAWVSSQEDISFSVMLGNINPPGTVKGFVAASLSTAGPDYFYSWVRDSALVSRVVTHKYNTTETGNSTVAGFLEDYVHFQINTQTEPTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSNKNYIIVTQDYQNGVQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSNEFASLAQNIASAADRFLSTVNYHRNSNGSLSEEFDRNTGYMTGARDLTWSHAAFITAGLARAGTPPASWGETSASSVPGTCAATSAIGTYSSVTVTSWPSIVATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 114 - protein, 622 aa, synthetic construct
  REGION 1..622: note = Chimeric protein
MMMSWKSLLLLLGPIAAATAAIPTGNSSLNAWVSSQEDISFSVMLGNINPPGTVKGFVAASLSTAGPDYFYSWVRDSALVSRVVTHKYNTTETGNSTVAGFLEDYVHFQINTQTEPTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSNKNYIIVTQDYQNGVQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSNEFASLAQNIASAADRFLSTVNYHRNSNGSLSEEFDRNTGYMTGARDLTWSHAAFITAGLARAGTPPSWANSSASTIPSTCSGASVVGSYSRPTATSFPPSQTPKPGVPSGTPYTPLPCATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 115 - protein, 462 aa, synthetic construct
  REGION 1..462: note = Chimeric protein
MMMSWKSLLLLLGPIAAATAAIPTGNSSLNAWVSSQEDISFSVMLGNINPPGTVKGFVAASLSTAGPDYFYSWVRDSALVSRVVTHKYNTTETGNSTVAGFLQDYVHFQINTQTEPTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIMVTQDYQNGIQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSDEFNSLAQNIASAADRFLSTVNYHRNSNGSLSEEYDRNSGYMTGARDLTWSHAAFITAGLARAGTPS

SEQ ID NO: 116 - protein, 607 aa, synthetic construct
  REGION 1..607: note = Chimeric protein
MKFSNLLKKPLLLIAGILAATVVAETVPTTAAVKVKSFTYDGSTFAGQIYVKNIAYTKTVTVIYSDASNNWNNNGNTIAASYSEGISGTNYEYWTFSAPVSGIKQFYVKYVVSGTTYYDNNNSGNYQVTTTTTTTAPTSTTSGGSSTTTGGSTTTATSVPTGVPSGFPTGNSSLNAWVSSQEDISFSVMLGNINPPGTVKGFVAASLSTAGPDYFYSWVRDSALVSRVVTHKYNTTETGNSTVAGFLQDYVHFQINTQTEPTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIMVTQDYQNGIQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSDEFNSLAQNIASAADRFLSTVNYHRNSNGSLSEEYDRNSGYMTGARDLTWSHAAFITAGLARAGTPS

SEQ ID NO: 117 - protein, 608 aa, synthetic construct
  REGION 1..608: note = Chimeric protein
MKFSKLFTKPTLFIVSIIISVASSETVPTTANVLVKSYTWDGATLSGQIYIKNLAYAKVVSVIYSDANDNWNNNGNKVAASYSAGIDGTNYEYWTFSGAVSGIKQFYVKYDVSGTSYYDNNGTKNYQVTKTSTTTTAATTTTTATTTTGGTGTTTTTTATATATSTDFPTGNSSLNAWVSSQEDISFSVMLGNINPPGTVKGFVAASLSTAGPDYFYSWVRDSALVSRVVTHKYNTTETGNSTVAGFLQDYVHFQINTQTEPTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIMVTQDYQNGIQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSDEFNSLAQNIASAADRFLSTVNYHRNSNGSLSEEYDRNSGYMTGARDLTWSHAAFITAGLARAGTPS

SEQ ID NO: 118 - protein, 644 aa, synthetic construct
  REGION 1..644: note = Chimeric protein
MMMSWKSLLLLLGPIAAATAAIPTGNSSLNAWVSSQEDISFSVMLGNINPPGTVKGFVAASLSTAGPDYFYSWVRDSALVSRVVTHKYNTTETGNSTVAGFLQDYVHFQINTQTEPTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIMVTQDYQNGIQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSDEFNSLAQNIASAADRFLSTVNYHRNSNGSLSEEYDRNSGYMTGARDLTWSHAAFITAGLARAGTPPASWGETSASSVPGTCAATSAIGTYSSVTVTSWPSIVATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 119 - protein, 622 aa, synthetic construct
  REGION 1..622: note = Chimeric protein
MMMSWKSLLLLLGPIAAATAAIPTGNSSLNAWVSSQEDISFSVMLGNINPPGTVKGFVAASLSTAGPDYFYSWVRDSALVSRVVTHKYNTTETGNSTVAGFLQDYVHFQINTQTEPTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIMVTQDYQNGIQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSDEFNSLAQNIASAADRFLSTVNYHRNSNGSLSEEYDRNSGYMTGARDLTWSHAAFITAGLARAGTPPSWANSSASTIPSTCSGASVVGSYSRPTATSFPPSQTPKPGVPSGTPYTPLPCATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 120 - protein, 463 aa, synthetic construct
  REGION 1..463: note = Chimeric protein
MMMSWKSLLLLLGPIAAATAAIPTGNSSLNAWVSSQEDISFSVMLGNINPPGTVKGFVAASLSTAGPDYFYSWVRDSALVSRVVTHKYNTTETGNSTVAGFLQDYVHFQINTQTEPTVCNCLGEPKFNPDGSSYTGPWGRPQNDGPAERASTMILIADSFLAQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIMVTQDYQNGVQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSDEFNSLAQNIASAADRFLSTVNYHRNSNGSLSEEYDRNSGYMTGARDLTWSHAAFITAGLARAGTPSF

SEQ ID NO: 121 - protein, 608 aa, synthetic construct
  REGION 1..608: note = Chimeric protein
MKFSNLLKKPLLLIAGILAATVVAETVPTTAAVKVKSFTYDGSTFAGQIYVKNIAYTKTVTVIYSDASNNWNNNGNTIAASYSEGISGTNYEYWTFSAPVSGIKQFYVKYVVSGTTYYDNNNSGNYQVTTTTTTTAPTSTTSGGSSTTTGGSTTTATSVPTGVPSGFPTGNSSLNAWVSSQEDISFSVMLGNINPPGTVKGFVAASLSTAGPDYFYSWVRDSALVSRVVTHKYNTTETGNSTVAGFLQDYVHFQINTQTEPTVCNCLGEPKFNPDGSSYTGPWGRPQNDGPAERASTMILIADSFLAQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIMVTQDYQNGVQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSDEFNSLAQNIASAADRFLSTVNYHRNSNGSLSEEYDRNSGYMTGARDLTWSHAAFITAGLARAGTPSF

SEQ ID NO: 122 - protein, 609 aa, synthetic construct
  REGION 1..609: note = Chimeric protein
MKFSKLFTKPTLFIVSIIISVASSETVPTTANVLVKSYTWDGATLSGQIYIKNLAYAKVVSVIYSDANDNWNNNGNKVAASYSAGIDGTNYEYWTFSGAVSGIKQFYVKYDVSGTSYYDNNGTKNYQVTKTSTTTTAATTTTTATTTTGGTGTTTTTTATATATSTDFPTGNSSLNAWVSSQEDISFSVMLGNINPPGTVKGFVAASLSTAGPDYFYSWVRDSALVSRVVTHKYNTTETGNSTVAGFLQDYVHFQINTQTEPTVCNCLGEPKFNPDGSSYTGPWGRPQNDGPAERASTMILIADSFLAQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIMVTQDYQNGVQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSDEFNSLAQNIASAADRFLSTVNYHRNSNGSLSEEYDRNSGYMTGARDLTWSHAAFITAGLARAGTPSF

SEQ ID NO: 123 - protein, 644 aa, synthetic construct
  REGION 1..644: note = Chimeric protein
MMMSWKSLLLLLGPIAAATAAIPTGNSSLNAWVSSQEDISFSVMLGNINPPGTVKGFVAASLSTAGPDYFYSWVRDSALVSRVVTHKYNTTETGNSTVAGFLQDYVHFQINTQTEPTVCNCLGEPKFNPDGSSYTGPWGRPQNDGPAERASTMILIADSFLAQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIMVTQDYQNGVQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSDEFNSLAQNIASAADRFLSTVNYHRNSNGSLSEEYDRNSGYMTGARDLTWSHAAFITAGLARAGTPPASWGETSASSVPGTCAATSAIGTYSSVTVTSWPSIVATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 124 - protein, 622 aa, synthetic construct
  REGION 1..622: note = Chimeric protein
MMMSWKSLLLLLGPIAAATAAIPTGNSSLNAWVSSQEDISFSVMLGNINPPGTVKGFVAASLSTAGPDYFYSWVRDSALVSRVVTHKYNTTETGNSTVAGFLQDYVHFQINTQTEPTVCNCLGEPKFNPDGSSYTGPWGRPQNDGPAERASTMILIADSFLAQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIMVTQDYQNGVQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSDEFNSLAQNIASAADRFLSTVNYHRNSNGSLSEEYDRNSGYMTGARDLTWSHAAFITAGLARAGTPPSWANSSASTIPSTCSGASVVGSYSRPTATSFPPSQTPKPGVPSGTPYTPLPCATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 125 - protein, 463 aa, synthetic construct
  REGION 1..463: note = Encodes chimeric protein
MMMSWKSLLLLLGPIAAATAAIPTGNSSLNAWVSSQEDISFSVMLGNINPPGTVKGFVAASLSTAGPDYFYSWVRDSALVSRVVTHKYNTTETGNSTVAGFLQDYVHFQINTQTEPTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIIVTQDYQNGVQKPSGLDVSTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSNEYASLAQNIASAADRFLSTVNYHRNPNGSLSEEYDRNTGYMTGARDLTWSHAAFITAGLARAGTPSF

SEQ ID NO: 126 - protein, 608 aa, synthetic construct
  REGION 1..608: note = Chimeric protein
MKFSNLLKKPLLLIAGILAATVVAETVPTTAAVKVKSFTYDGSTFAGQIYVKNIAYTKTVTVIYSDASNNWNNNGNTIAASYSEGISGTNYEYWTFSAPVSGIKQFYVKYVVSGTTYYDNNNSGNYQVTTTTTTTAPTSTTSGGSSTTTGGSTTTATSVPTGVPSGFPTGNSSLNAWVSSQEDISFSVMLGNINPPGTVKGFVAASLSTAGPDYFYSWVRDSALVSRVVTHKYNTTETGNSTVAGFLQDYVHFQINTQTEPTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIIVTQDYQNGVQKPSGLDVSTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSNEYASLAQNIASAADRFLSTVNYHRNPNGSLSEEYDRNTGYMTGARDLTWSHAAFITAGLARAGTPSF

SEQ ID NO: 127 - protein, 609 aa, synthetic construct
  REGION 1..609: note = Chimeric protein
MKFSKLFTKPTLFIVSIIISVASSETVPTTANVLVKSYTWDGATLSGQIYIKNLAYAKVVSVIYSDANDNWNNNGNKVAASYSAGIDGTNYEYWTFSGAVSGIKQFYVKYDVSGTSYYDNNGTKNYQVTKTSTTTTAATTTTTATTTTGGTGTTTTTTATATATSTDFPTGNSSLNAWVSSQEDISFSVMLGNINPPGTVKGFVAASLSTAGPDYFYSWVRDSALVSRVVTHKYNTTETGNSTVAGFLQDYVHFQINTQTEPTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIIVTQDYQNGVQKPSGLDVSTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSNEYASLAQNIASAADRFLSTVNYHRNPNGSLSEEYDRNTGYMTGARDLTWSHAAFITAGLARAGTPSF

SEQ ID NO: 128 - protein, 644 aa, synthetic construct
  REGION 1..644: note = Chimeric protein
MMMSWKSLLLLLGPIAAATAAIPTGNSSLNAWVSSQEDISFSVMLGNINPPGTVKGFVAASLSTAGPDYFYSWVRDSALVSRVVTHKYNTTETGNSTVAGFLQDYVHFQINTQTEPTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIIVTQDYQNGVQKPSGLDVSTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSNEYASLAQNIASAADRFLSTVNYHRNPNGSLSEEYDRNTGYMTGARDLTWSHAAFITAGLARAGTPPASWGETSASSVPGTCAATSAIGTYSSVTVTSWPSIVATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 129 - protein, 622 aa, synthetic construct
  REGION 1..622: note = Chimeric protein
MMMSWKSLLLLLGPIAAATAAIPTGNSSLNAWVSSQEDISFSVMLGNINPPGTVKGFVAASLSTAGPDYFYSWVRDSALVSRVVTHKYNTTETGNSTVAGFLQDYVHFQINTQTEPTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIIVTQDYQNGVQKPSGLDVSTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSNEYASLAQNIASAADRFLSTVNYHRNPNGSLSEEYDRNTGYMTGARDLTWSHAAFITAGLARAGTPPSWANSSASTIPSTCSGASVVGSYSRPTATSFPPSQTPKPGVPSGTPYTPLPCATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 130 - DNA, 1404 bp, synthetic construct
  misc_feature 1..1404: note = Encodes chimeric protein
  source 1..1404: mol_type = other DNA
atgccttcttggaagaccctgattttcctcctgggccctattaccgccgctctggctgctcctgttaacaagcagtctttccctaccggcaaccccgacatcagctcttgggtttctaagcaggagggcattagcttcagcgagatgctccgaaacgtcaaccctcccggcaccgctaagggcttcgttgctgctagcctcagcaccgccggccccgactacttctacacctggacccgagacgccgctctcgtctcccgcgtcatcgcttacaagtacaacaccaccaacgctggcgacaacaacattcacggcgctctgcaggactacgtcaccttccagattaacacccagaccgagcccaccccttgcaactgcctgggcgagcctaagttcaaccccgacggcagctctttcaccggcccttggggccgacctcagaacgacggccctgctgagcgcgctagcagcatgattctcattgctgacagcttcctcgcccagaccaaggacaccgcttacgttaccaacaccctgaagcctgccatttacaaggacctggactacgttgttaacacctggtctaacccttgcttcgacctgtgggaggaggttaacggcgtccacttctacaccctcatggttatgcgacgaggcctcctcgacggcgctgacttcgctacccgaaacggcgacgcctctaaggctagctcttactctgacgctgctagcaagatcaagacccgactggactctttctgggtcagcgacaagaactacattaccgttacccaggactacaaggagggcgtcaagaaggacaccggcctcgacgttagcaccctcattgctgccaacgttgccggcaaggacgacggcttctacacccccggcagcgacaagattctggctaccgctgttgccttcgagaacgccatggctaagctgtaccctctgaaccagaacctcgacagccacctgggcaactctattggccgataccctgaggacacctacaacggcaacggcaactcccagggcaacccttggttcctcgccaccaccgctttcaccgagctgtactaccgagccatcttggaatggaaggacagcggcgttaccgtcacccccgtttccaaggacttcttcgccaagttcgactccaacgccgttgccggcaagaagtactctcctaactctgaggagttcgccaccctgaccgctaacatcgctgctgccgctgaccgattcatgtctaccgttaagtaccaccagaaccctaacggctctctgtccgagcagttcgaccgccacagcggctacatgaccggcgcccgagacctgacctggtctcacgctgctttcattaccgccgcccaggctcgcgctggcacccctagcttc

SEQ ID NO: 131 - DNA, 1824 bp, synthetic construct
  misc_feature 1..1824: note = Encodes chimeric protein
  source 1..1824: mol_type = other DNA
atgaagttctctaacctcctgaagaagcccctcctgctcattgctggcattctcgctgccaccgtcgttgccgagaccgtccctaccaccgctgctgttaaggttaagtctttcacctacgacggcagcaccttcgctggccagatttacgtcaagaacatcgcttacaccaagaccgttaccgttatttactctgacgcctctaacaactggaacaacaacggcaacaccattgccgcctcttacagcgagggcatctctggcaccaactacgagtactggaccttcagcgcccctgtttccggcatcaagcagttctacgtcaagtacgttgtcagcggcaccacctactacgacaacaacaactctggcaactaccaggttaccaccaccaccaccaccaccgcccctaccagcaccacctccggcggctctagcaccaccaccggcggctccaccaccaccgccacctctgtccctaccggcgtccctagcggcttccctaccggcaaccccgacatcagctcttgggtttctaagcaggagggcattagcttcagcgagatgctccgaaacgtcaaccctcccggcaccgctaagggcttcgttgctgctagcctcagcaccgccggccccgactacttctacacctggacccgagacgccgctctcgtctcccgcgtcatcgcttacaagtacaacaccaccaacgctggcgacaacaacattcacggcgctctgcaggactacgtcaccttccagattaacacccagaccgagcccaccccttgcaactgcctgggcgagcctaagttcaaccccgacggcagctctttcaccggcccttggggccgacctcagaacgacggccctgctgagcgcgctagcagcatgattctcattgctgacagcttcctcgcccagaccaaggacaccgcttacgttaccaacaccctgaagcctgccatttacaaggacctggactacgttgttaacacctggtctaacccttgcttcgacctgtgggaggaggttaacggcgtccacttctacaccctcatggttatgcgacgaggcctcctcgacggcgctgacttcgctacccgaaacggcgacgcctctaaggctagctcttactctgacgctgctagcaagatcaagacccgactggactctttctgggtcagcgacaagaactacattaccgttacccaggactacaaggagggcgtcaagaaggacaccggcctcgacgttagcaccctcattgctgccaacgttgccggcaaggacgacggcttctacacccccggcagcgacaagattctggctaccgctgttgccttcgagaacgccatggctaagctgtaccctctgaaccagaacctcgacagccacctgggcaactctattggccgataccctgaggacacctacaacggcaacggcaactcccagggcaacccttggttcctcgccaccaccgctttcaccgagctgtactaccgagccatcttggaatggaaggacagcggcgttaccgtcacccccgtttccaaggacttcttcgccaagttcgactccaacgccgttgccggcaagaagtactctcctaactctgaggagttcgccaccctgaccgctaacatcgctgctgccgctgaccgattcatgtctaccgttaagtaccaccagaaccctaacggctctctgtccgagcagttcgaccgccacagcggctacatgaccggcgcccgagacctgacctggtctcacgctgctttcattaccgccgcccaggctcgcgctggcacccctagcttc

SEQ ID NO: 132 - DNA, 1827 bp, synthetic construct
  misc_feature 1..1827: note = Encodes chimeric protein
  source 1..1827: mol_type = other DNA
atgaagttcagcaagctgttcaccaagcctaccctgttcatcgtttctattattatcagcgttgcttctagcgagaccgtccctaccaccgctaacgtcctcgttaagtcttacacctgggacggcgctaccctgtctggccagatttacattaagaacctcgcttacgccaaggttgttagcgttatttactctgacgctaacgacaactggaacaacaacggcaacaaggttgccgcttcttactctgctggcattgacggcaccaactacgagtactggaccttcagcggcgccgtttccggcatcaagcagttctacgtcaagtacgacgtcagcggcacctcttactacgacaacaacggcaccaagaactaccaggttaccaagacctctaccaccaccaccgccgctaccaccaccaccaccgctaccaccaccaccggcggcaccggcaccaccaccaccaccaccgccaccgccaccgctaccagcaccgacttccctaccggcaaccccgacatcagctcttgggtttctaagcaggagggcattagcttcagcgagatgctccgaaacgtcaaccctcccggcaccgctaagggcttcgttgctgctagcctcagcaccgccggccccgactacttctacacctggacccgagacgccgctctcgtctcccgcgtcatcgcttacaagtacaacaccaccaacgctggcgacaacaacattcacggcgctctgcaggactacgtcaccttccagattaacacccagaccgagcccaccccttgcaactgcctgggcgagcctaagttcaaccccgacggcagctctttcaccggcccttggggccgacctcagaacgacggccctgctgagcgcgctagcagcatgattctcattgctgacagcttcctcgcccagaccaaggacaccgcttacgttaccaacaccctgaagcctgccatttacaaggacctggactacgttgttaacacctggtctaacccttgcttcgacctgtgggaggaggttaacggcgtccacttctacaccctcatggttatgcgacgaggcctcctcgacggcgctgacttcgctacccgaaacggcgacgcctctaaggctagctcttactctgacgctgctagcaagatcaagacccgactggactctttctgggtcagcgacaagaactacattaccgttacccaggactacaaggagggcgtcaagaaggacaccggcctcgacgttagcaccctcattgctgccaacgttgccggcaaggacgacggcttctacacccccggcagcgacaagattctggctaccgctgttgccttcgagaacgccatggctaagctgtaccctctgaaccagaacctcgacagccacctgggcaactctattggccgataccctgaggacacctacaacggcaacggcaactcccagggcaacccttggttcctcgccaccaccgctttcaccgagctgtactaccgagccatcttggaatggaaggacagcggcgttaccgtcacccccgtttccaaggacttcttcgccaagttcgactccaacgccgttgccggcaagaagtactctcctaactctgaggagttcgccaccctgaccgctaacatcgctgctgccgctgaccgattcatgtctaccgttaagtaccaccagaaccctaacggctctctgtccgagcagttcgaccgccacagcggctacatgaccggcgcccgagacctgacctggtctcacgctgctttcattaccgccgcccaggctcgcgctggcacccctagcttc

SEQ ID NO: 133 - DNA, 1947 bp, synthetic construct
  misc_feature 1..1947: note = Encodes chimeric protein
  source 1..1947: mol_type = other DNA
atgccttcttggaagaccctgattttcctcctgggccctattaccgccgctctggctgctcctgttaacaagcagtctttccctaccggcaaccccgacatcagctcttgggtttctaagcaggagggcattagcttcagcgagatgctccgaaacgtcaaccctcccggcaccgctaagggcttcgttgctgctagcctcagcaccgccggccccgactacttctacacctggacccgagacgccgctctcgtctcccgcgtcatcgcttacaagtacaacaccaccaacgctggcgacaacaacattcacggcgctctgcaggactacgtcaccttccagattaacacccagaccgagcccaccccttgcaactgcctgggcgagcctaagttcaaccccgacggcagctctttcaccggcccttggggccgacctcagaacgacggccctgctgagcgcgctagcagcatgattctcattgctgacagcttcctcgcccagaccaaggacaccgcttacgttaccaacaccctgaagcctgccatttacaaggacctggactacgttgttaacacctggtctaacccttgcttcgacctgtgggaggaggttaacggcgtccacttctacaccctcatggttatgcgacgaggcctcctcgacggcgctgacttcgctacccgaaacggcgacgcctctaaggctagctcttactctgacgctgctagcaagatcaagacccgactggactctttctgggtcagcgacaagaactacattaccgttacccaggactacaaggagggcgtcaagaaggacaccggcctcgacgttagcaccctcattgctgccaacgttgccggcaaggacgacggcttctacacccccggcagcgacaagattctggctaccgctgttgccttcgagaacgccatggctaagctgtaccctctgaaccagaacctcgacagccacctgggcaactctattggccgataccctgaggacacctacaacggcaacggcaactcccagggcaacccttggttcctcgccaccaccgctttcaccgagctgtactaccgagccatcttggaatggaaggacagcggcgttaccgtcacccccgtttccaaggacttcttcgccaagttcgactccaacgccgttgccggcaagaagtactctcctaactctgaggagttcgccaccctgaccgctaacatcgctgctgccgctgaccgattcatgtctaccgttaagtaccaccagaaccctaacggctctctgtccgagcagttcgaccgccacagcggctacatgaccggcgcccgagacctgacctggtctcacgctgctttcattaccgccgcccaggctcgcgctggcacccctcctgcttcttggggcgagacctccgcttctagcgtccccggcacctgcgctgctaccagcgctattggcacctactctagcgtcaccgttacctcttggccttccattgttgctaccggcggcaccaccaccaccgccacccctaccggctctggctctgttaccagcaccagcaagaccaccgctaccgcttctaagacctccacctctacctctagcaccagctgcaccacccctaccgccgtcgccgttaccttcgacctgaccgctaccaccacctacggcgagaacatttacctggttggctccattagccagctgggcgactgggagacctccgacggcatcgccctgtctgctgacaagtacaccagctccgaccctctgtggtacgttaccgttaccctgcccgccggcgagtctttcgagtacaagttcatccgaattgagtctgacgactctgttgagtgggagagcgaccccaaccgagagtacaccgttcctcaggcttgcggcaccagcaccgctaccgtcaccgacacctggcgc

SEQ ID NO: 134 - DNA, 1881 bp, synthetic construct
  misc_feature 1..1881: note = Encodes chimeric protein
  source 1..1881: mol_type = other DNA
atgccttcttggaagaccctgattttcctcctgggccctattaccgccgctctggctgctcctgttaacaagcagtctttccctaccggcaaccccgacatcagctcttgggtttctaagcaggagggcattagcttcagcgagatgctccgaaacgtcaaccctcccggcaccgctaagggcttcgttgctgctagcctcagcaccgccggccccgactacttctacacctggacccgagacgccgctctcgtctcccgcgtcatcgcttacaagtacaacaccaccaacgctggcgacaacaacattcacggcgctctgcaggactacgtcaccttccagattaacacccagaccgagcccaccccttgcaactgcctgggcgagcctaagttcaaccccgacggcagctctttcaccggcccttggggccgacctcagaacgacggccctgctgagcgcgctagcagcatgattctcattgctgacagcttcctcgcccagaccaaggacaccgcttacgttaccaacaccctgaagcctgccatttacaaggacctggactacgttgttaacacctggtctaacccttgcttcgacctgtgggaggaggttaacggcgtccacttctacaccctcatggttatgcgacgaggcctcctcgacggcgctgacttcgctacccgaaacggcgacgcctctaaggctagctcttactctgacgctgctagcaagatcaagacccgactggactctttctgggtcagcgacaagaactacattaccgttacccaggactacaaggagggcgtcaagaaggacaccggcctcgacgttagcaccctcattgctgccaacgttgccggcaaggacgacggcttctacacccccggcagcgacaagattctggctaccgctgttgccttcgagaacgccatggctaagctgtaccctctgaaccagaacctcgacagccacctgggcaactctattggccgataccctgaggacacctacaacggcaacggcaactcccagggcaacccttggttcctcgccaccaccgctttcaccgagctgtactaccgagccatcttggaatggaaggacagcggcgttaccgtcacccccgtttccaaggacttcttcgccaagttcgactccaacgccgttgccggcaagaagtactctcctaactctgaggagttcgccaccctgaccgctaacatcgctgctgccgctgaccgattcatgtctaccgttaagtaccaccagaaccctaacggctctctgtccgagcagttcgaccgccacagcggctacatgaccggcgcccgagacctgacctggtctcacgctgctttcattaccgccgcccaggctcgcgctggcacccctccttcttgggctaacagctctgcttctaccattcctagcacctgcagcggcgctagcgtcgttggctcttactcccgacctaccgctacctctttccctccttctcagacccctaagcccggcgttcctagcggcaccccttacacccctctgccttgcgccacccctaccagcgtcgccgttaccttccacgagctcgtttctacccagttcggccagaccgtcaaggttgctggcaacgccgctgccctgggcaactggagcaccagcgccgctgttgctctcgacgctgttaactacgctgacaaccaccctctgtggattggcaccgttaacttagaagccggcgacgttgttgagtacaagtacatcaacgtcggccaggacggctccgttacctgggagtccgaccccaaccacacctacaccgttcctgctgtcgcttgcgtcacccaggttgtcaaggaggacacctggcagtct

SEQ ID NO: 135 - DNA, 1389 bp, synthetic construct
  misc_feature 1..1389: note = Encodes chimeric protein
  source 1..1389: mol_type = other DNA
atgatgatgtcttggaagtctctgctcctcctgctgggccctatcgccgctgctaccgctgccatccctaccggcaacagcagcctgaacgcttgggttagctcccaggaggacatctctttctccgtcatgctgggcaacattaaccctcccggcaccgtcaagggcttcgttgccgctagcctgtccaccgctggccccgactacttctactcttgggtccgagacagcgccctcgtttcccgcgtcgtcacccacaagtacaacaccaccgagaccggcaactctaccgttgccggcttcttggaggactacgttcacttccagattaacacccagaccgagcccaccgtttgcaactgcctgggcgagcctaagttcaaccccgacggcagctcttacaccggcccttggggccgccctcagaacgacggccccgctgagcgagctagcaccatgattctgattgccgactctttcctgacccagaccaagaacacctcttacgttgacaacaccctgaagcccgccatttacaaggacctggactacgttgttaacacctggtctaacccttgcttcgacctgtgggaggaggttaacggcattcacttctacaccctcatggttatgcgacgaggcctcctcgacggcgccaacttcgccacccgaaacggcgacacctctaaggctagctcttacagcagcaccgctagccagattcagaacaagattgactccttctggagctccaacaagaactacattattgttacccaggactaccagaacggcgtccagaagcctagcggcctcgacatcagcaccctcattgctgccaacgtcgctggcatgaacgacggcttctacacccccggctctgacaagatgctcgccaccgccgttgctattgagaacgccatggctaacctgtaccccctcaacaagaacctgccttcttacctgggcaacgctattggccgataccctgaggacacctacaacggcgacggcaactcccagggcaacccttggttcctcgccaccaccgctttcagcgagctgtactaccgagctctgctggagtggcaggagaccggcgttaccgttaccagcatttctaaggacttcttcagcaagttcgaccctaacgctagcgctggcaagaagtactctcccggctctaacgagttcgcttctctggctcagaacattgctagcgctgctgaccgattcctgtccaccgtcaactaccaccgaaactccaacggcagcctgtctgaggagttcgaccgaaacaccggctacatgaccggcgcccgagacctgacctggtctcacgccgctttcattaccgctggcctcgctcgcgctggcacccctagcttc

SEQ ID NO: 136 - DNA, 1824 bp, synthetic construct
  misc_feature 1..1824: note = Encodes chimeric protein
  source 1..1824: mol_type = other DNA
atgaagttctctaacctcctgaagaagcccctcctgctcattgctggcattctcgctgccaccgtcgttgccgagaccgtccctaccaccgctgctgttaaggttaagtctttcacctacgacggcagcaccttcgctggccagatttacgtcaagaacatcgcttacaccaagaccgttaccgttatttactctgacgcctctaacaactggaacaacaacggcaacaccattgccgcctcttacagcgagggcatctctggcaccaactacgagtactggaccttcagcgcccctgtttccggcatcaagcagttctacgtcaagtacgttgtcagcggcaccacctactacgacaacaacaactctggcaactaccaggttaccaccaccaccaccaccaccgcccctaccagcaccacctccggcggctctagcaccaccaccggcggctccaccaccaccgccacctctgtccctaccggcgtccctagcggcttccctaccggcaacagcagcctgaacgcttgggttagctcccaggaggacatctctttctccgtcatgctgggcaacattaaccctcccggcaccgtcaagggcttcgttgccgctagcctgtccaccgctggccccgactacttctactcttgggtccgagacagcgccctcgtttcccgcgtcgtcacccacaagtacaacaccaccgagaccggcaactctaccgttgccggcttcttggaggactacgttcacttccagattaacacccagaccgagcccaccgtttgcaactgcctgggcgagcctaagttcaaccccgacggcagctcttacaccggcccttggggccgccctcagaacgacggccccgctgagcgagctagcaccatgattctgattgccgactctttcctgacccagaccaagaacacctcttacgttgacaacaccctgaagcccgccatttacaaggacctggactacgttgttaacacctggtctaacccttgcttcgacctgtgggaggaggttaacggcattcacttctacaccctcatggttatgcgacgaggcctcctcgacggcgccaacttcgccacccgaaacggcgacacctctaaggctagctcttacagcagcaccgctagccagattcagaacaagattgactccttctggagctccaacaagaactacattattgttacccaggactaccagaacggcgtccagaagcctagcggcctcgacatcagcaccctcattgctgccaacgtcgctggcatgaacgacggcttctacacccccggctctgacaagatgctcgccaccgccgttgctattgagaacgccatggctaacctgtaccccctcaacaagaacctgccttcttacctgggcaacgctattggccgataccctgaggacacctacaacggcgacggcaactcccagggcaacccttggttcctcgccaccaccgctttcagcgagctgtactaccgagctctgctggagtggcaggagaccggcgttaccgttaccagcatttctaaggacttcttcagcaagttcgaccctaacgctagcgctggcaagaagtactctcccggctctaacgagttcgcttctctggctcagaacattgctagcgctgctgaccgattcctgtccaccgtcaactaccaccgaaactccaacggcagcctgtctgaggagttcgaccgaaacaccggctacatgaccggcgcccgagacctgacctggtctcacgccgctttcattaccgctggcctcgctcgcgctggcacccctagcttc

SEQ ID NO: 137 - DNA, 1827 bp, synthetic construct
  misc_feature 1..1827: note = Encodes chimeric sequence
  source 1..1827: mol_type = other DNA
atgaagttcagcaagctgttcaccaagcctaccctgttcatcgtttctattattatcagcgttgcttctagcgagaccgtccctaccaccgctaacgtcctcgttaagtcttacacctgggacggcgctaccctgtctggccagatttacattaagaacctcgcttacgccaaggttgttagcgttatttactctgacgctaacgacaactggaacaacaacggcaacaaggttgccgcttcttactctgctggcattgacggcaccaactacgagtactggaccttcagcggcgccgtttccggcatcaagcagttctacgtcaagtacgacgtcagcggcacctcttactacgacaacaacggcaccaagaactaccaggttaccaagacctctaccaccaccaccgccgctaccaccaccaccaccgctaccaccaccaccggcggcaccggcaccaccaccaccaccaccgccaccgccaccgctaccagcaccgacttccctaccggcaacagcagcctgaacgcttgggttagctcccaggaggacatctctttctccgtcatgctgggcaacattaaccctcccggcaccgtcaagggcttcgttgccgctagcctgtccaccgctggccccgactacttctactcttgggtccgagacagcgccctcgtttcccgcgtcgtcacccacaagtacaacaccaccgagaccggcaactctaccgttgccggcttcttggaggactacgttcacttccagattaacacccagaccgagcccaccgtttgcaactgcctgggcgagcctaagttcaaccccgacggcagctcttacaccggcccttggggccgccctcagaacgacggccccgctgagcgagctagcaccatgattctgattgccgactctttcctgacccagaccaagaacacctcttacgttgacaacaccctgaagcccgccatttacaaggacctggactacgttgttaacacctggtctaacccttgcttcgacctgtgggaggaggttaacggcattcacttctacaccctcatggttatgcgacgaggcctcctcgacggcgccaacttcgccacccgaaacggcgacacctctaaggctagctcttacagcagcaccgctagccagattcagaacaagattgactccttctggagctccaacaagaactacattattgttacccaggactaccagaacggcgtccagaagcctagcggcctcgacatcagcaccctcattgctgccaacgtcgctggcatgaacgacggcttctacacccccggctctgacaagatgctcgccaccgccgttgctattgagaacgccatggctaacctgtaccccctcaacaagaacctgccttcttacctgggcaacgctattggccgataccctgaggacacctacaacggcgacggcaactcccagggcaacccttggttcctcgccaccaccgctttcagcgagctgtactaccgagctctgctggagtggcaggagaccggcgttaccgttaccagcatttctaaggacttcttcagcaagttcgaccctaacgctagcgctggcaagaagtactctcccggctctaacgagttcgcttctctggctcagaacattgctagcgctgctgaccgattcctgtccaccgtcaactaccaccgaaactccaacggcagcctgtctgaggagttcgaccgaaacaccggctacatgaccggcgcccgagacctgacctggtctcacgccgctttcattaccgctggcctcgctcgcgctggcacccctagcttc

SEQ ID NO: 138 - DNA, 1932 bp, synthetic construct
  misc_feature 1..1932: note = Encodes chimeric protein
  source 1..1932: mol_type = other DNA
atgatgatgtcttggaagtctctgctcctcctgctgggccctatcgccgctgctaccgctgccatccctaccggcaacagcagcctgaacgcttgggttagctcccaggaggacatctctttctccgtcatgctgggcaacattaaccctcccggcaccgtcaagggcttcgttgccgctagcctgtccaccgctggccccgactacttctactcttgggtccgagacagcgccctcgtttcccgcgtcgtcacccacaagtacaacaccaccgagaccggcaactctaccgttgccggcttcttggaggactacgttcacttccagattaacacccagaccgagcccaccgtttgcaactgcctgggcgagcctaagttcaaccccgacggcagctcttacaccggcccttggggccgccctcagaacgacggccccgctgagcgagctagcaccatgattctgattgccgactctttcctgacccagaccaagaacacctcttacgttgacaacaccctgaagcccgccatttacaaggacctggactacgttgttaacacctggtctaacccttgcttcgacctgtgggaggaggttaacggcattcacttctacaccctcatggttatgcgacgaggcctcctcgacggcgccaacttcgccacccgaaacggcgacacctctaaggctagctcttacagcagcaccgctagccagattcagaacaagattgactccttctggagctccaacaagaactacattattgttacccaggactaccagaacggcgtccagaagcctagcggcctcgacatcagcaccctcattgctgccaacgtcgctggcatgaacgacggcttctacacccccggctctgacaagatgctcgccaccgccgttgctattgagaacgccatggctaacctgtaccccctcaacaagaacctgccttcttacctgggcaacgctattggccgataccctgaggacacctacaacggcgacggcaactcccagggcaacccttggttcctcgccaccaccgctttcagcgagctgtactaccgagctctgctggagtggcaggagaccggcgttaccgttaccagcatttctaaggacttcttcagcaagttcgaccctaacgctagcgctggcaagaagtactctcccggctctaacgagttcgcttctctggctcagaacattgctagcgctgctgaccgattcctgtccaccgtcaactaccaccgaaactccaacggcagcctgtctgaggagttcgaccgaaacaccggctacatgaccggcgcccgagacctgacctggtctcacgccgctttcattaccgctggcctcgctcgcgctggcacccctcctgcttcttggggcgagacctccgcttctagcgtccccggcacctgcgctgctaccagcgctattggcacctactctagcgtcaccgttacctcttggccttccattgttgctaccggcggcaccaccaccaccgccacccctaccggctctggctctgttaccagcaccagcaagaccaccgctaccgcttctaagacctccacctctacctctagcaccagctgcaccacccctaccgccgtcgccgttaccttcgacctgaccgctaccaccacctacggcgagaacatttacctggttggctccattagccagctgggcgactgggagacctccgacggcatcgccctgtctgctgacaagtacaccagctccgaccctctgtggtacgttaccgttaccctgcccgccggcgagtctttcgagtacaagttcatccgaattgagtctgacgactctgttgagtgggagagcgaccccaaccgagagtacaccgttcctcaggcttgcggcaccagcaccgctaccgtcaccgacacctggcgc

SEQ ID NO: 139 - DNA, 1866 bp, synthetic construct
  misc_feature 1..1866: note = Encodes chimeric protein
  source 1..1866: mol_type = other DNA
atgatgatgtcttggaagtctctgctcctcctgctgggccctatcgccgctgctaccgctgccatccctaccggcaacagcagcctgaacgcttgggttagctcccaggaggacatctctttctccgtcatgctgggcaacattaaccctcccggcaccgtcaagggcttcgttgccgctagcctgtccaccgctggccccgactacttctactcttgggtccgagacagcgccctcgtttcccgcgtcgtcacccacaagtacaacaccaccgagaccggcaactctaccgttgccggcttcttggaggactacgttcacttccagattaacacccagaccgagcccaccgtttgcaactgcctgggcgagcctaagttcaaccccgacggcagctcttacaccggcccttggggccgccctcagaacgacggccccgctgagcgagctagcaccatgattctgattgccgactctttcctgacccagaccaagaacacctcttacgttgacaacaccctgaagcccgccatttacaaggacctggactacgttgttaacacctggtctaacccttgcttcgacctgtgggaggaggttaacggcattcacttctacaccctcatggttatgcgacgaggcctcctcgacggcgccaacttcgccacccgaaacggcgacacctctaaggctagctcttacagcagcaccgctagccagattcagaacaagattgactccttctggagctccaacaagaactacattattgttacccaggactaccagaacggcgtccagaagcctagcggcctcgacatcagcaccctcattgctgccaacgtcgctggcatgaacgacggcttctacacccccggctctgacaagatgctcgccaccgccgttgctattgagaacgccatggctaacctgtaccccctcaacaagaacctgccttcttacctgggcaacgctattggccgataccctgaggacacctacaacggcgacggcaactcccagggcaacccttggttcctcgccaccaccgctttcagcgagctgtactaccgagctctgctggagtggcaggagaccggcgttaccgttaccagcatttctaaggacttcttcagcaagttcgaccctaacgctagcgctggcaagaagtactctcccggctctaacgagttcgcttctctggctcagaacattgctagcgctgctgaccgattcctgtccaccgtcaactaccaccgaaactccaacggcagcctgtctgaggagttcgaccgaaacaccggctacatgaccggcgcccgagacctgacctggtctcacgccgctttcattaccgctggcctcgctcgcgctggcacccctccttcttgggctaacagctctgcttctaccattcctagcacctgcagcggcgctagcgtcgttggctcttactcccgacctaccgctacctctttccctccttctcagacccctaagcccggcgttcctagcggcaccccttacacccctctgccttgcgccacccctaccagcgtcgccgttaccttccacgagctcgtttctacccagttcggccagaccgtcaaggttgctggcaacgccgctgccctgggcaactggagcaccagcgccgctgttgctctcgacgctgttaactacgctgacaaccaccctctgtggattggcaccgttaacttagaagccggcgacgttgttgagtacaagtacatcaacgtcggccaggacggctccgttacctgggagtccgaccccaaccacacctacaccgttcctgctgtcgcttgcgtcacccaggttgtcaaggaggacacctggcagtct

SEQ ID NO: 140 - DNA, 1386 bp, synthetic construct
  misc_feature 1..1386: note = Encodes chimeric protein
  source 1..1386: mol_type = other DNA
atgatgatgtcttggaagtctctgctcctgctcctgggccctattgctgccgccaccgctgccatccctaccggcaactctagcctcaacgcttgggttagcagccaggaggacatttctttcagcgtcatgctgggcaacattaaccctcccggcaccgtcaagggcttcgttgctgctagcctgtccaccgctggccccgactacttctactcttgggttcgagacagcgctctcgtttctcgcgtcgttacccacaagtacaacaccaccgagaccggcaacagcaccgttgctggcttcctgcaggactacgttcacttccagattaacacccagaccgagcccaccgtttgcaactgcctgggcgagcctaagttcaaccccgacggcagctcttacaccggcccttggggccgccctcagaacgacggccccgctgagcgagctagcaccatgattctgattgctgactccttcctgacccagaccaagaacacctcttacgttgacaacaccctgaagcccgctatttacaaggacctggactacgtcgtcaacacctggtctaacccttgcttcgacctgtgggaggaggttaacggcattcacttctacaccctcatggttatgcgacgaggcctcctggacggcgccaacttcgccacccgaaacggcgacaccagcaaggctagctcttacagcagcaccgcttctcagatccagaacaagattgactctttctggtctagctctaagaactacatcatggttacccaggactaccagaacggcattcagaagcctagcggcctcgacattagcaccctcatcgccgctaacgtcgctggcatgaacgacggcttctacacccccggctccgacaagatgctcgctaccgccgttgctattgagaacgccatggctaacctgtaccctctgaacaagaacctgccttcttacctgggcaacgccattggccgataccccgaggacacctacaacggcgacggcaactctcagggcaacccttggttcctcgccaccaccgctttcagcgagctgtactaccgcgctctcctggagtggcaggagaccggcgttaccgtcaccagcatttccaaggacttcttctctaagttcgaccctaacgctagcgctggcaagaagtactcccccggctctgacgagttcaactccctcgcccagaacattgccagcgctgccgaccgattcctgtctaccgttaactaccaccgaaactccaacggctctctgtctgaggagtacgaccgaaacagcggctacatgaccggcgcccgagacctgacctggtcccacgccgctttcattaccgccggcctcgcgagagctggcacccctagc

SEQ ID NO: 141 - DNA, 1821 bp, synthetic construct
  misc_feature 1..1821: note = Encodes chimeric protein
  source 1..1821: mol_type = other DNA
atgaagttctctaacctcctgaagaagcccctcctgctcattgctggcattctcgctgccaccgtcgttgccgagaccgtccctaccaccgctgctgttaaggttaagtctttcacctacgacggcagcaccttcgctggccagatttacgtcaagaacatcgcttacaccaagaccgttaccgttatttactctgacgcctctaacaactggaacaacaacggcaacaccattgccgcctcttacagcgagggcatctctggcaccaactacgagtactggaccttcagcgcccctgtttccggcatcaagcagttctacgtcaagtacgttgtcagcggcaccacctactacgacaacaacaactctggcaactaccaggttaccaccaccaccaccaccaccgcccctaccagcaccacctccggcggctctagcaccaccaccggcggctccaccaccaccgccacctctgtccctaccggcgtccctagcggcttccctaccggcaactctagcctcaacgcttgggttagcagccaggaggacatttctttcagcgtcatgctgggcaacattaaccctcccggcaccgtcaagggcttcgttgctgctagcctgtccaccgctggccccgactacttctactcttgggttcgagacagcgctctcgtttctcgcgtcgttacccacaagtacaacaccaccgagaccggcaacagcaccgttgctggcttcctgcaggactacgttcacttccagattaacacccagaccgagcccaccgtttgcaactgcctgggcgagcctaagttcaaccccgacggcagctcttacaccggcccttggggccgccctcagaacgacggccccgctgagcgagctagcaccatgattctgattgctgactccttcctgacccagaccaagaacacctcttacgttgacaacaccctgaagcccgctatttacaaggacctggactacgtcgtcaacacctggtctaacccttgcttcgacctgtgggaggaggttaacggcattcacttctacaccctcatggttatgcgacgaggcctcctggacggcgccaacttcgccacccgaaacggcgacaccagcaaggctagctcttacagcagcaccgcttctcagatccagaacaagattgactctttctggtctagctctaagaactacatcatggttacccaggactaccagaacggcattcagaagcctagcggcctcgacattagcaccctcatcgccgctaacgtcgctggcatgaacgacggcttctacacccccggctccgacaagatgctcgctaccgccgttgctattgagaacgccatggctaacctgtaccctctgaacaagaacctgccttcttacctgggcaacgccattggccgataccccgaggacacctacaacggcgacggcaactctcagggcaacccttggttcctcgccaccaccgctttcagcgagctgtactaccgcgctctcctggagtggcaggagaccggcgttaccgtcaccagcatttccaaggacttcttctctaagttcgaccctaacgctagcgctggcaagaagtactcccccggctctgacgagttcaactccctcgcccagaacattgccagcgctgccgaccgattcctgtctaccgttaactaccaccgaaactccaacggctctctgtctgaggagtacgaccgaaacagcggctacatgaccggcgcccgagacctgacctggtcccacgccgctttcattaccgccggcctcgcgagagctggcacccctagc

SEQ ID NO: 142 - DNA, 1824 bp, synthetic construct
  misc_feature 1..1824: note = Encodes chimeric protein
  source 1..1824: mol_type = other DNA
atgaagttcagcaagctgttcaccaagcctaccctgttcatcgtttctattattatcagcgttgcttctagcgagaccgtccctaccaccgctaacgtcctcgttaagtcttacacctgggacggcgctaccctgtctggccagatttacattaagaacctcgcttacgccaaggttgttagcgttatttactctgacgctaacgacaactggaacaacaacggcaacaaggttgccgcttcttactctgctggcattgacggcaccaactacgagtactggaccttcagcggcgccgtttccggcatcaagcagttctacgtcaagtacgacgtcagcggcacctcttactacgacaacaacggcaccaagaactaccaggttaccaagacctctaccaccaccaccgccgctaccaccaccaccaccgctaccaccaccaccggcggcaccggcaccaccaccaccaccaccgccaccgccaccgctaccagcaccgacttccctaccggcaactctagcctcaacgcttgggttagcagccaggaggacatttctttcagcgtcatgctgggcaacattaaccctcccggcaccgtcaagggcttcgttgctgctagcctgtccaccgctggccccgactacttctactcttgggttcgagacagcgctctcgtttctcgcgtcgttacccacaagtacaacaccaccgagaccggcaacagcaccgttgctggcttcctgcaggactacgttcacttccagattaacacccagaccgagcccaccgtttgcaactgcctgggcgagcctaagttcaaccccgacggcagctcttacaccggcccttggggccgccctcagaacgacggccccgctgagcgagctagcaccatgattctgattgctgactccttcctgacccagaccaagaacacctcttacgttgacaacaccctgaagcccgctatttacaaggacctggactacgtcgtcaacacctggtctaacccttgcttcgacctgtgggaggaggttaacggcattcacttctacaccctcatggttatgcgacgaggcctcctggacggcgccaacttcgccacccgaaacggcgacaccagcaaggctagctcttacagcagcaccgcttctcagatccagaacaagattgactctttctggtctagctctaagaactacatcatggttacccaggactaccagaacggcattcagaagcctagcggcctcgacattagcaccctcatcgccgctaacgtcgctggcatgaacgacggcttctacacccccggctccgacaagatgctcgctaccgccgttgctattgagaacgccatggctaacctgtaccctctgaacaagaacctgccttcttacctgggcaacgccattggccgataccccgaggacacctacaacggcgacggcaactctcagggcaacccttggttcctcgccaccaccgctttcagcgagctgtactaccgcgctctcctggagtggcaggagaccggcgttaccgtcaccagcatttccaaggacttcttctctaagttcgaccctaacgctagcgctggcaagaagtactcccccggctctgacgagttcaactccctcgcccagaacattgccagcgctgccgaccgattcctgtctaccgttaactaccaccgaaactccaacggctctctgtctgaggagtacgaccgaaacagcggctacatgaccggcgcccgagacctgacctggtcccacgccgctttcattaccgccggcctcgcgagagctggcacccctagc

SEQ ID NO: 143 - DNA, 1932 bp, synthetic construct
  misc_feature 1..1932: note = Encodes chimeric protein
  source 1..1932: mol_type = other DNA
atgatgatgtcttggaagtctctgctcctgctcctgggccctattgctgccgccaccgctgccatccctaccggcaactctagcctcaacgcttgggttagcagccaggaggacatttctttcagcgtcatgctgggcaacattaaccctcccggcaccgtcaagggcttcgttgctgctagcctgtccaccgctggccccgactacttctactcttgggttcgagacagcgctctcgtttctcgcgtcgttacccacaagtacaacaccaccgagaccggcaacagcaccgttgctggcttcctgcaggactacgttcacttccagattaacacccagaccgagcccaccgtttgcaactgcctgggcgagcctaagttcaaccccgacggcagctcttacaccggcccttggggccgccctcagaacgacggccccgctgagcgagctagcaccatgattctgattgctgactccttcctgacccagaccaagaacacctcttacgttgacaacaccctgaagcccgctatttacaaggacctggactacgtcgtcaacacctggtctaacccttgcttcgacctgtgggaggaggttaacggcattcacttctacaccctcatggttatgcgacgaggcctcctggacggcgccaacttcgccacccgaaacggcgacaccagcaaggctagctcttacagcagcaccgcttctcagatccagaacaagattgactctttctggtctagctctaagaactacatcatggttacccaggactaccagaacggcattcagaagcctagcggcctcgacattagcaccctcatcgccgctaacgtcgctggcatgaacgacggcttctacacccccggctccgacaagatgctcgctaccgccgttgctattgagaacgccatggctaacctgtaccctctgaacaagaacctgccttcttacctgggcaacgccattggccgataccccgaggacacctacaacggcgacggcaactctcagggcaacccttggttcctcgccaccaccgctttcagcgagctgtactaccgcgctctcctggagtggcaggagaccggcgttaccgtcaccagcatttccaaggacttcttctctaagttcgaccctaacgctagcgctggcaagaagtactcccccggctctgacgagttcaactccctcgcccagaacattgccagcgctgccgaccgattcctgtctaccgttaactaccaccgaaactccaacggctctctgtctgaggagtacgaccgaaacagcggctacatgaccggcgcccgagacctgacctggtcccacgccgctttcattaccgccggcctcgcgagagctggcacccctcctgcttcttggggcgagacctccgcttctagcgtccccggcacctgcgctgctaccagcgctattggcacctactctagcgtcaccgttacctcttggccttccattgttgctaccggcggcaccaccaccaccgccacccctaccggctctggctctgttaccagcaccagcaagaccaccgctaccgcttctaagacctccacctctacctctagcaccagctgcaccacccctaccgccgtcgccgttaccttcgacctgaccgctaccaccacctacggcgagaacatttacctggttggctccattagccagctgggcgactgggagacctccgacggcatcgccctgtctgctgacaagtacaccagctccgaccctctgtggtacgttaccgttaccctgcccgccggcgagtctttcgagtacaagttcatccgaattgagtctgacgactctgttgagtgggagagcgaccccaaccgagagtacaccgttcctcaggcttgcggcaccagcaccgctaccgtcaccgacacctggcgc

SEQ ID NO: 144 - DNA, 1866 bp, synthetic construct
  misc_feature 1..1866: note = Encodes chimeric protein
  source 1..1866: mol_type = other DNA
atgatgatgtcttggaagtctctgctcctgctcctgggccctattgctgccgccaccgctgccatccctaccggcaactctagcctcaacgcttgggttagcagccaggaggacatttctttcagcgtcatgctgggcaacattaaccctcccggcaccgtcaagggcttcgttgctgctagcctgtccaccgctggccccgactacttctactcttgggttcgagacagcgctctcgtttctcgcgtcgttacccacaagtacaacaccaccgagaccggcaacagcaccgttgctggcttcctgcaggactacgttcacttccagattaacacccagaccgagcccaccgtttgcaactgcctgggcgagcctaagttcaaccccgacggcagctcttacaccggcccttggggccgccctcagaacgacggccccgctgagcgagctagcaccatgattctgattgctgactccttcctgacccagaccaagaacacctcttacgttgacaacaccctgaagcccgctatttacaaggacctggactacgtcgtcaacacctggtctaacccttgcttcgacctgtgggaggaggttaacggcattcacttctacaccctcatggttatgcgacgaggcctcctggacggcgccaacttcgccacccgaaacggcgacaccagcaaggctagctcttacagcagcaccgcttctcagatccagaacaagattgactctttctggtctagctctaagaactacatcatggttacccaggactaccagaacggcattcagaagcctagcggcctcgacattagcaccctcatcgccgctaacgtcgctggcatgaacgacggcttctacacccccggctccgacaagatgctcgctaccgccgttgctattgagaacgccatggctaacctgtaccctctgaacaagaacctgccttcttacctgggcaacgccattggccgataccccgaggacacctacaacggcgacggcaactctcagggcaacccttggttcctcgccaccaccgctttcagcgagctgtactaccgcgctctcctggagtggcaggagaccggcgttaccgtcaccagcatttccaaggacttcttctctaagttcgaccctaacgctagcgctggcaagaagtactcccccggctctgacgagttcaactccctcgcccagaacattgccagcgctgccgaccgattcctgtctaccgttaactaccaccgaaactccaacggctctctgtctgaggagtacgaccgaaacagcggctacatgaccggcgcccgagacctgacctggtcccacgccgctttcattaccgccggcctcgcgagagctggcacccctccttcttgggctaacagctctgcttctaccattcctagcacctgcagcggcgctagcgtcgttggctcttactcccgacctaccgctacctctttccctccttctcagacccctaagcccggcgttcctagcggcaccccttacacccctctgccttgcgccacccctaccagcgtcgccgttaccttccacgagctcgtttctacccagttcggccagaccgtcaaggttgctggcaacgccgctgccctgggcaactggagcaccagcgccgctgttgctctcgacgctgttaactacgctgacaaccaccctctgtggattggcaccgttaacttagaagccggcgacgttgttgagtacaagtacatcaacgtcggccaggacggctccgttacctgggagtccgaccccaaccacacctacaccgttcctgctgtcgcttgcgtcacccaggttgtcaaggaggacacctggcagtct

SEQ ID NO: 145 - DNA, 1389 bp, synthetic construct
  misc_feature 1..1389: note = Encodes chimeric protein
  source 1..1389: mol_type = other DNA
atgatgatgtcttggaagtctctgctcctcctgctgggccctattgccgctgccaccgctgccattcctaccggcaacagcagcctcaacgcttgggttagctcccaggaggacatttctttcagcgtcatgctgggcaacattaaccctcccggcaccgttaagggcttcgttgccgctagcctgtctaccgccggccccgactacttctactcttgggtccgagacagcgccctcgtttcccgcgtcgtcacccacaagtacaacaccaccgagaccggcaacagcaccgttgctggcttcctgcaggactacgtccacttccagattaacacccagaccgagcccaccgtttgcaactgcctgggcgagcctaagttcaaccccgacggcagctcttacaccggcccttggggccgacctcagaacgacggccccgctgagcgcgctagcaccatgattctgattgctgactccttcctcgctcagaccaagaacacctcttacgttgacaacaccctgaagcctgccatttacaaggacctggactacgtcgttaacacctggtctaacccttgcttcgacctgtgggaggaggttaacggcattcacttctacaccctcatggttatgcgacgaggcctcctggacggcgccaacttcgccacccgaaacggcgacacctccaaggctagctcttacagcagcaccgctagccagatccagaacaagattgactctttctggtctagctccaagaactacatcatggttacccaggactaccagaacggcgttcagaagcctagcggcctcgacattagcaccctcatcgccgctaacgttgctggcatgaacgacggcttctacacccctggctctgacaagatgctcgccaccgccgttgctattgagaacgctatggctaacctgtaccctctgaacaagaacctgccttcttacctgggcaacgccattggccgataccccgaggacacctacaacggcgacggcaactcccagggcaacccttggttcctcgctaccaccgctttcagcgagctgtactaccgagctctgttagagtggcaggagaccggcgtcaccgttaccagcatctctaaggacttcttctccaagttcgaccctaacgctagcgccggcaagaagtactcccccggcagcgacgagttcaactctctcgcccagaacatcgccagcgccgctgaccgattcctgtctaccgtcaactaccaccgaaactctaacggctctctgtctgaggagtacgaccgaaactccggctacatgaccggcgcgagagacctgacctggtctcacgctgctttcattaccgctggcctcgcccgagctggcaccccttctttc

SEQ ID NO: 146 - DNA, 1824 bp, synthetic construct
  misc_feature 1..1824: note = Encodes chimeric protein
  source 1..1824: mol_type = other DNA
atgaagttctctaacctcctgaagaagcccctcctgctcattgctggcattctcgctgccaccgtcgttgccgagaccgtccctaccaccgctgctgttaaggttaagtctttcacctacgacggcagcaccttcgctggccagatttacgtcaagaacatcgcttacaccaagaccgttaccgttatttactctgacgcctctaacaactggaacaacaacggcaacaccattgccgcctcttacagcgagggcatctctggcaccaactacgagtactggaccttcagcgcccctgtttccggcatcaagcagttctacgtcaagtacgttgtcagcggcaccacctactacgacaacaacaactctggcaactaccaggttaccaccaccaccaccaccaccgcccctaccagcaccacctccggcggctctagcaccaccaccggcggctccaccaccaccgccacctctgtccctaccggcgtccctagcggcttccctaccggcaacagcagcctcaacgcttgggttagctcccaggaggacatttctttcagcgtcatgctgggcaacattaaccctcccggcaccgttaagggcttcgttgccgctagcctgtctaccgccggccccgactacttctactcttgggtccgagacagcgccctcgtttcccgcgtcgtcacccacaagtacaacaccaccgagaccggcaacagcaccgttgctggcttcctgcaggactacgtccacttccagattaacacccagaccgagcccaccgtttgcaactgcctgggcgagcctaagttcaaccccgacggcagctcttacaccggcccttggggccgacctcagaacgacggccccgctgagcgcgctagcaccatgattctgattgctgactccttcctcgctcagaccaagaacacctcttacgttgacaacaccctgaagcctgccatttacaaggacctggactacgtcgttaacacctggtctaacccttgcttcgacctgtgggaggaggttaacggcattcacttctacaccctcatggttatgcgacgaggcctcctggacggcgccaacttcgccacccgaaacggcgacacctccaaggctagctcttacagcagcaccgctagccagatccagaacaagattgactctttctggtctagctccaagaactacatcatggttacccaggactaccagaacggcgttcagaagcctagcggcctcgacattagcaccctcatcgccgctaacgttgctggcatgaacgacggcttctacacccctggctctgacaagatgctcgccaccgccgttgctattgagaacgctatggctaacctgtaccctctgaacaagaacctgccttcttacctgggcaacgccattggccgataccccgaggacacctacaacggcgacggcaactcccagggcaacccttggttcctcgctaccaccgctttcagcgagctgtactaccgagctctgttagagtggcaggagaccggcgtcaccgttaccagcatctctaaggacttcttctccaagttcgaccctaacgctagcgccggcaagaagtactcccccggcagcgacgagttcaactctctcgcccagaacatcgccagcgccgctgaccgattcctgtctaccgtcaactaccaccgaaactctaacggctctctgtctgaggagtacgaccgaaactccggctacatgaccggcgcgagagacctgacctggtctcacgctgctttcattaccgctggcctcgcccgagctggcaccccttctttc

SEQ ID NO: 147 - DNA, 1827 bp, synthetic construct
  misc_feature 1..1827: note = Encodes chimeric protein
  source 1..1827: mol_type = other DNA
atgaagttcagcaagctgttcaccaagcctaccctgttcatcgtttctattattatcagcgttgcttctagcgagaccgtccctaccaccgctaacgtcctcgttaagtcttacacctgggacggcgctaccctgtctggccagatttacattaagaacctcgcttacgccaaggttgttagcgttatttactctgacgctaacgacaactggaacaacaacggcaacaaggttgccgcttcttactctgctggcattgacggcaccaactacgagtactggaccttcagcggcgccgtttccggcatcaagcagttctacgtcaagtacgacgtcagcggcacctcttactacgacaacaacggcaccaagaactaccaggttaccaagacctctaccaccaccaccgccgctaccaccaccaccaccgctaccaccaccaccggcggcaccggcaccaccaccaccaccaccgccaccgccaccgctaccagcaccgacttccctaccggcaacagcagcctcaacgcttgggttagctcccaggaggacatttctttcagcgtcatgctgggcaacattaaccctcccggcaccgttaagggcttcgttgccgctagcctgtctaccgccggccccgactacttctactcttgggtccgagacagcgccctcgtttcccgcgtcgtcacccacaagtacaacaccaccgagaccggcaacagcaccgttgctggcttcctgcaggactacgtccacttccagattaacacccagaccgagcccaccgtttgcaactgcctgggcgagcctaagttcaaccccgacggcagctcttacaccggcccttggggccgacctcagaacgacggccccgctgagcgcgctagcaccatgattctgattgctgactccttcctcgctcagaccaagaacacctcttacgttgacaacaccctgaagcctgccatttacaaggacctggactacgtcgttaacacctggtctaacccttgcttcgacctgtgggaggaggttaacggcattcacttctacaccctcatggttatgcgacgaggcctcctggacggcgccaacttcgccacccgaaacggcgacacctccaaggctagctcttacagcagcaccgctagccagatccagaacaagattgactctttctggtctagctccaagaactacatcatggttacccaggactaccagaacggcgttcagaagcctagcggcctcgacattagcaccctcatcgccgctaacgttgctggcatgaacgacggcttctacacccctggctctgacaagatgctcgccaccgccgttgctattgagaacgctatggctaacctgtaccctctgaacaagaacctgccttcttacctgggcaacgccattggccgataccccgaggacacctacaacggcgacggcaactcccagggcaacccttggttcctcgctaccaccgctttcagcgagctgtactaccgagctctgttagagtggcaggagaccggcgtcaccgttaccagcatctctaaggacttcttctccaagttcgaccctaacgctagcgccggcaagaagtactcccccggcagcgacgagttcaactctctcgcccagaacatcgccagcgccgctgaccgattcctgtctaccgtcaactaccaccgaaactctaacggctctctgtctgaggagtacgaccgaaactccggctacatgaccggcgcgagagacctgacctggtctcacgctgctttcattaccgctggcctcgcccgagctggcaccccttctttc

SEQ ID NO: 148 - DNA, 1932 bp, synthetic construct
  misc_feature 1..1932: note = Encodes chimeric protein
  source 1..1932: mol_type = other DNA
atgatgatgtcttggaagtctctgctcctcctgctgggccctattgccgctgccaccgctgccattcctaccggcaacagcagcctcaacgcttgggttagctcccaggaggacatttctttcagcgtcatgctgggcaacattaaccctcccggcaccgttaagggcttcgttgccgctagcctgtctaccgccggccccgactacttctactcttgggtccgagacagcgccctcgtttcccgcgtcgtcacccacaagtacaacaccaccgagaccggcaacagcaccgttgctggcttcctgcaggactacgtccacttccagattaacacccagaccgagcccaccgtttgcaactgcctgggcgagcctaagttcaaccccgacggcagctcttacaccggcccttggggccgacctcagaacgacggccccgctgagcgcgctagcaccatgattctgattgctgactccttcctcgctcagaccaagaacacctcttacgttgacaacaccctgaagcctgccatttacaaggacctggactacgtcgttaacacctggtctaacccttgcttcgacctgtgggaggaggttaacggcattcacttctacaccctcatggttatgcgacgaggcctcctggacggcgccaacttcgccacccgaaacggcgacacctccaaggctagctcttacagcagcaccgctagccagatccagaacaagattgactctttctggtctagctccaagaactacatcatggttacccaggactaccagaacggcgttcagaagcctagcggcctcgacattagcaccctcatcgccgctaacgttgctggcatgaacgacggcttctacacccctggctctgacaagatgctcgccaccgccgttgctattgagaacgctatggctaacctgtaccctctgaacaagaacctgccttcttacctgggcaacgccattggccgataccccgaggacacctacaacggcgacggcaactcccagggcaacccttggttcctcgctaccaccgctttcagcgagctgtactaccgagctctgttagagtggcaggagaccggcgtcaccgttaccagcatctctaaggacttcttctccaagttcgaccctaacgctagcgccggcaagaagtactcccccggcagcgacgagttcaactctctcgcccagaacatcgccagcgccgctgaccgattcctgtctaccgtcaactaccaccgaaactctaacggctctctgtctgaggagtacgaccgaaactccggctacatgaccggcgcgagagacctgacctggtctcacgctgctttcattaccgctggcctcgcccgagctggcacccctcctgcttcttggggcgagacctccgcttctagcgtccccggcacctgcgctgctaccagcgctattggcacctactctagcgtcaccgttacctcttggccttccattgttgctaccggcggcaccaccaccaccgccacccctaccggctctggctctgttaccagcaccagcaagaccaccgctaccgcttctaagacctccacctctacctctagcaccagctgcaccacccctaccgccgtcgccgttaccttcgacctgaccgctaccaccacctacggcgagaacatttacctggttggctccattagccagctgggcgactgggagacctccgacggcatcgccctgtctgctgacaagtacaccagctccgaccctctgtggtacgttaccgttaccctgcccgccggcgagtctttcgagtacaagttcatccgaattgagtctgacgactctgttgagtgggagagcgaccccaaccgagagtacaccgttcctcaggcttgcggcaccagcaccgctaccgtcaccgacacctggcgc

SEQ ID NO: 149 - DNA, 1866 bp, synthetic construct
  misc_feature 1..1866: note = Encodes chimeric protein
  source 1..1866: mol_type = other DNA
atgatgatgtcttggaagtctctgctcctcctgctgggccctattgccgctgccaccgctgccattcctaccggcaacagcagcctcaacgcttgggttagctcccaggaggacatttctttcagcgtcatgctgggcaacattaaccctcccggcaccgttaagggcttcgttgccgctagcctgtctaccgccggccccgactacttctactcttgggtccgagacagcgccctcgtttcccgcgtcgtcacccacaagtacaacaccaccgagaccggcaacagcaccgttgctggcttcctgcaggactacgtccacttccagattaacacccagaccgagcccaccgtttgcaactgcctgggcgagcctaagttcaaccccgacggcagctcttacaccggcccttggggccgacctcagaacgacggccccgctgagcgcgctagcaccatgattctgattgctgactccttcctcgctcagaccaagaacacctcttacgttgacaacaccctgaagcctgccatttacaaggacctggactacgtcgttaacacctggtctaacccttgcttcgacctgtgggaggaggttaacggcattcacttctacaccctcatggttatgcgacgaggcctcctggacggcgccaacttcgccacccgaaacggcgacacctccaaggctagctcttacagcagcaccgctagccagatccagaacaagattgactctttctggtctagctccaagaactacatcatggttacccaggactaccagaacggcgttcagaagcctagcggcctcgacattagcaccctcatcgccgctaacgttgctggcatgaacgacggcttctacacccctggctctgacaagatgctcgccaccgccgttgctattgagaacgctatggctaacctgtaccctctgaacaagaacctgccttcttacctgggcaacgccattggccgataccccgaggacacctacaacggcgacggcaactcccagggcaacccttggttcctcgctaccaccgctttcagcgagctgtactaccgagctctgttagagtggcaggagaccggcgtcaccgttaccagcatctctaaggacttcttctccaagttcgaccctaacgctagcgccggcaagaagtactcccccggcagcgacgagttcaactctctcgcccagaacatcgccagcgccgctgaccgattcctgtctaccgtcaactaccaccgaaactctaacggctctctgtctgaggagtacgaccgaaactccggctacatgaccggcgcgagagacctgacctggtctcacgctgctttcattaccgctggcctcgcccgagctggcacccctccttcttgggctaacagctctgcttctaccattcctagcacctgcagcggcgctagcgtcgttggctcttactcccgacctaccgctacctctttccctccttctcagacccctaagcccggcgttcctagcggcaccccttacacccctctgccttgcgccacccctaccagcgtcgccgttaccttccacgagctcgtttctacccagttcggccagaccgtcaaggttgctggcaacgccgctgccctgggcaactggagcaccagcgccgctgttgctctcgacgctgttaactacgctgacaaccaccctctgtggattggcaccgttaacttagaagccggcgacgttgttgagtacaagtacatcaacgtcggccaggacggctccgttacctgggagtccgaccccaaccacacctacaccgttcctgctgtcgcttgcgtcacccaggttgtcaaggaggacacctggcagtct

SEQ ID NO: 150 - DNA, 1389 bp, synthetic construct
  misc_feature 1..1389: note = Encodes chimeric protein
  source 1..1389: mol_type = other DNA
atgatgatgtcttggaagtctctgctcctcctgctgggccctatcgccgctgccaccgctgccatccctaccggcaacagcagcctcaacgcttgggttagctctcaggaggacatctctttcagcgtcatgctgggcaacatcaaccctcccggcaccgtcaagggcttcgttgccgctagcctgtctaccgctggccccgactacttctactcttgggtccgagacagcgctctcgtttctcgcgttgttacccacaagtacaacaccaccgagaccggcaacagcaccgttgctggcttcctgcaggactacgttcacttccagattaacacccagaccgagcccaccgtttgcaactgcctgggcgagcctaagttcaaccccgacggcagctcttacaccggcccttggggccgccctcagaacgacggccccgctgagcgcgctagcaccatgattctgatcgctgactccttcctgacccagaccaagaacacctcttacgttgacaacaccctgaagcccgctatttacaaggacctcgactacgtcgttaacacctggtctaacccttgcttcgacctgtgggaggaggttaacggcattcacttctacaccctcatggttatgcgacgaggcctcctggacggcgccaacttcgccacccgaaacggcgacaccagcaaggctagctcttactccagcaccgcttcccagattcagaacaagattgactctttctggagcagctccaagaactacattattgttacccaggactaccagaacggcgtccagaagcctagcggcctcgacgtcagcaccctcattgctgccaacgtcgctggcatgaacgacggcttctacacccccggcagcgacaagatgctcgccaccgccgttgccattgagaacgccatggctaacctgtaccctctgaacaagaacctgccttcttacctgggcaacgctattggccgataccccgaggacacctacaacggcgacggcaactcccagggcaacccttggttcctcgccaccaccgctttcagcgagctgtactaccgagctctgcttgaatggcaggagaccggcgtcaccgttaccagcatttctaaggacttcttctctaagttcgaccctaacgctagcgccggcaagaagtactcccccggctccaacgagtacgctagcctcgcccagaacattgctagcgccgctgaccgattcctgtctaccgttaactaccaccgaaaccctaacggctctctgtctgaggagtacgaccgaaacaccggctacatgaccggcgcccgagacctgacctggtctcacgctgctttcattaccgccggcctggccagggctggcaccccttctttc

SEQ ID NO: 151 - DNA, 1824 bp, synthetic construct
  misc_feature 1..1824: note = Encodes chimeric protein
  source 1..1824: mol_type = other DNA
atgaagttctctaacctcctgaagaagcccctcctgctcattgctggcattctcgctgccaccgtcgttgccgagaccgtccctaccaccgctgctgttaaggttaagtctttcacctacgacggcagcaccttcgctggccagatttacgtcaagaacatcgcttacaccaagaccgttaccgttatttactctgacgcctctaacaactggaacaacaacggcaacaccattgccgcctcttacagcgagggcatctctggcaccaactacgagtactggaccttcagcgcccctgtttccggcatcaagcagttctacgtcaagtacgttgtcagcggcaccacctactacgacaacaacaactctggcaactaccaggttaccaccaccaccaccaccaccgcccctaccagcaccacctccggcggctctagcaccaccaccggcggctccaccaccaccgccacctctgtccctaccggcgtccctagcggcttccctaccggcaacagcagcctcaacgcttgggttagctctcaggaggacatctctttcagcgtcatgctgggcaacatcaaccctcccggcaccgtcaagggcttcgttgccgctagcctgtctaccgctggccccgactacttctactcttgggtccgagacagcgctctcgtttctcgcgttgttacccacaagtacaacaccaccgagaccggcaacagcaccgttgctggcttcctgcaggactacgttcacttccagattaacacccagaccgagcccaccgtttgcaactgcctgggcgagcctaagttcaaccccgacggcagctcttacaccggcccttggggccgccctcagaacgacggccccgctgagcgcgctagcaccatgattctgatcgctgactccttcctgacccagaccaagaacacctcttacgttgacaacaccctgaagcccgctatttacaaggacctcgactacgtcgttaacacctggtctaacccttgcttcgacctgtgggaggaggttaacggcattcacttctacaccctcatggttatgcgacgaggcctcctggacggcgccaacttcgccacccgaaacggcgacaccagcaaggctagctcttactccagcaccgcttcccagattcagaacaagattgactctttctggagcagctccaagaactacattattgttacccaggactaccagaacggcgtccagaagcctagcggcctcgacgtcagcaccctcattgctgccaacgtcgctggcatgaacgacggcttctacacccccggcagcgacaagatgctcgccaccgccgttgccattgagaacgccatggctaacctgtaccctctgaacaagaacctgccttcttacctgggcaacgctattggccgataccccgaggacacctacaacggcgacggcaactcccagggcaacccttggttcctcgccaccaccgctttcagcgagctgtactaccgagctctgcttgaatggcaggagaccggcgtcaccgttaccagcatttctaaggacttcttctctaagttcgaccctaacgctagcgccggcaagaagtactcccccggctccaacgagtacgctagcctcgcccagaacattgctagcgccgctgaccgattcctgtctaccgttaactaccaccgaaaccctaacggctctctgtctgaggagtacgaccgaaacaccggctacatgaccggcgcccgagacctgacctggtctcacgctgctttcattaccgccggcctggccagggctggcaccccttctttc

SEQ ID NO: 152 - DNA, 1827 bp, synthetic construct
  misc_feature 1..1827: note = Encodes chimeric protein
  source 1..1827: mol_type = other DNA
atgaagttcagcaagctgttcaccaagcctaccctgttcatcgtttctattattatcagcgttgcttctagcgagaccgtccctaccaccgctaacgtcctcgttaagtcttacacctgggacggcgctaccctgtctggccagatttacattaagaacctcgcttacgccaaggttgttagcgttatttactctgacgctaacgacaactggaacaacaacggcaacaaggttgccgcttcttactctgctggcattgacggcaccaactacgagtactggaccttcagcggcgccgtttccggcatcaagcagttctacgtcaagtacgacgtcagcggcacctcttactacgacaacaacggcaccaagaactaccaggttaccaagacctctaccaccaccaccgccgctaccaccaccaccaccgctaccaccaccaccggcggcaccggcaccaccaccaccaccaccgccaccgccaccgctaccagcaccgacttccctaccggcaacagcagcctcaacgcttgggttagctctcaggaggacatctctttcagcgtcatgctgggcaacatcaaccctcccggcaccgtcaagggcttcgttgccgctagcctgtctaccgctggccccgactacttctactcttgggtccgagacagcgctctcgtttctcgcgttgttacccacaagtacaacaccaccgagaccggcaacagcaccgttgctggcttcctgcaggactacgttcacttccagattaacacccagaccgagcccaccgtttgcaactgcctgggcgagcctaagttcaaccccgacggcagctcttacaccggcccttggggccgccctcagaacgacggccccgctgagcgcgctagcaccatgattctgatcgctgactccttcctgacccagaccaagaacacctcttacgttgacaacaccctgaagcccgctatttacaaggacctcgactacgtcgttaacacctggtctaacccttgcttcgacctgtgggaggaggttaacggcattcacttctacaccctcatggttatgcgacgaggcctcctggacggcgccaacttcgccacccgaaacggcgacaccagcaaggctagctcttactccagcaccgcttcccagattcagaacaagattgactctttctggagcagctccaagaactacattattgttacccaggactaccagaacggcgtccagaagcctagcggcctcgacgtcagcaccctcattgctgccaacgtcgctggcatgaacgacggcttctacacccccggcagcgacaagatgctcgccaccgccgttgccattgagaacgccatggctaacctgtaccctctgaacaagaacctgccttcttacctgggcaacgctattggccgataccccgaggacacctacaacggcgacggcaactcccagggcaacccttggttcctcgccaccaccgctttcagcgagctgtactaccgagctctgcttgaatggcaggagaccggcgtcaccgttaccagcatttctaaggacttcttctctaagttcgaccctaacgctagcgccggcaagaagtactcccccggctccaacgagtacgctagcctcgcccagaacattgctagcgccgctgaccgattcctgtctaccgttaactaccaccgaaaccctaacggctctctgtctgaggagtacgaccgaaacaccggctacatgaccggcgcccgagacctgacctggtctcacgctgctttcattaccgccggcctggccagggctggcaccccttctttc

SEQ ID NO: 153 - DNA, 1932 bp, synthetic construct
  misc_feature 1..1932: note = Encodes chimeric protein
  source 1..1932: mol_type = other DNA
atgatgatgtcttggaagtctctgctcctcctgctgggccctatcgccgctgccaccgctgccatccctaccggcaacagcagcctcaacgcttgggttagctctcaggaggacatctctttcagcgtcatgctgggcaacatcaaccctcccggcaccgtcaagggcttcgttgccgctagcctgtctaccgctggccccgactacttctactcttgggtccgagacagcgctctcgtttctcgcgttgttacccacaagtacaacaccaccgagaccggcaacagcaccgttgctggcttcctgcaggactacgttcacttccagattaacacccagaccgagcccaccgtttgcaactgcctgggcgagcctaagttcaaccccgacggcagctcttacaccggcccttggggccgccctcagaacgacggccccgctgagcgcgctagcaccatgattctgatcgctgactccttcctgacccagaccaagaacacctcttacgttgacaacaccctgaagcccgctatttacaaggacctcgactacgtcgttaacacctggtctaacccttgcttcgacctgtgggaggaggttaacggcattcacttctacaccctcatggttatgcgacgaggcctcctggacggcgccaacttcgccacccgaaacggcgacaccagcaaggctagctcttactccagcaccgcttcccagattcagaacaagattgactctttctggagcagctccaagaactacattattgttacccaggactaccagaacggcgtccagaagcctagcggcctcgacgtcagcaccctcattgctgccaacgtcgctggcatgaacgacggcttctacacccccggcagcgacaagatgctcgccaccgccgttgccattgagaacgccatggctaacctgtaccctctgaacaagaacctgccttcttacctgggcaacgctattggccgataccccgaggacacctacaacggcgacggcaactcccagggcaacccttggttcctcgccaccaccgctttcagcgagctgtactaccgagctctgcttgaatggcaggagaccggcgtcaccgttaccagcatttctaaggacttcttctctaagttcgaccctaacgctagcgccggcaagaagtactcccccggctccaacgagtacgctagcctcgcccagaacattgctagcgccgctgaccgattcctgtctaccgttaactaccaccgaaaccctaacggctctctgtctgaggagtacgaccgaaacaccggctacatgaccggcgcccgagacctgacctggtctcacgctgctttcattaccgccggcctggccagggctggcacccctcctgcttcttggggcgagacctccgcttctagcgtccccggcacctgcgctgctaccagcgctattggcacctactctagcgtcaccgttacctcttggccttccattgttgctaccggcggcaccaccaccaccgccacccctaccggctctggctctgttaccagcaccagcaagaccaccgctaccgcttctaagacctccacctctacctctagcaccagctgcaccacccctaccgccgtcgccgttaccttcgacctgaccgctaccaccacctacggcgagaacatttacctggttggctccattagccagctgggcgactgggagacctccgacggcatcgccctgtctgctgacaagtacaccagctccgaccctctgtggtacgttaccgttaccctgcccgccggcgagtctttcgagtacaagttcatccgaattgagtctgacgactctgttgagtgggagagcgaccccaaccgagagtacaccgttcctcaggcttgcggcaccagcaccgctaccgtcaccgacacctggcgc

SEQ ID NO: 154 - DNA, 1866 bp, synthetic construct
  misc_feature 1..1866: note = Encodes chimeric protein
  source 1..1866: mol_type = other DNA
atgatgatgtcttggaagtctctgctcctcctgctgggccctatcgccgctgccaccgctgccatccctaccggcaacagcagcctcaacgcttgggttagctctcaggaggacatctctttcagcgtcatgctgggcaacatcaaccctcccggcaccgtcaagggcttcgttgccgctagcctgtctaccgctggccccgactacttctactcttgggtccgagacagcgctctcgtttctcgcgttgttacccacaagtacaacaccaccgagaccggcaacagcaccgttgctggcttcctgcaggactacgttcacttccagattaacacccagaccgagcccaccgtttgcaactgcctgggcgagcctaagttcaaccccgacggcagctcttacaccggcccttggggccgccctcagaacgacggccccgctgagcgcgctagcaccatgattctgatcgctgactccttcctgacccagaccaagaacacctcttacgttgacaacaccctgaagcccgctatttacaaggacctcgactacgtcgttaacacctggtctaacccttgcttcgacctgtgggaggaggttaacggcattcacttctacaccctcatggttatgcgacgaggcctcctggacggcgccaacttcgccacccgaaacggcgacaccagcaaggctagctcttactccagcaccgcttcccagattcagaacaagattgactctttctggagcagctccaagaactacattattgttacccaggactaccagaacggcgtccagaagcctagcggcctcgacgtcagcaccctcattgctgccaacgtcgctggcatgaacgacggcttctacacccccggcagcgacaagatgctcgccaccgccgttgccattgagaacgccatggctaacctgtaccctctgaacaagaacctgccttcttacctgggcaacgctattggccgataccccgaggacacctacaacggcgacggcaactcccagggcaacccttggttcctcgccaccaccgctttcagcgagctgtactaccgagctctgcttgaatggcaggagaccggcgtcaccgttaccagcatttctaaggacttcttctctaagttcgaccctaacgctagcgccggcaagaagtactcccccggctccaacgagtacgctagcctcgcccagaacattgctagcgccgctgaccgattcctgtctaccgttaactaccaccgaaaccctaacggctctctgtctgaggagtacgaccgaaacaccggctacatgaccggcgcccgagacctgacctggtctcacgctgctttcattaccgccggcctggccagggctggcacccctccttcttgggctaacagctctgcttctaccattcctagcacctgcagcggcgctagcgtcgttggctcttactcccgacctaccgctacctctttccctccttctcagacccctaagcccggcgttcctagcggcaccccttacacccctctgccttgcgccacccctaccagcgtcgccgttaccttccacgagctcgtttctacccagttcggccagaccgtcaaggttgctggcaacgccgctgccctgggcaactggagcaccagcgccgctgttgctctcgacgctgttaactacgctgacaaccaccctctgtggattggcaccgttaacttagaagccggcgacgttgttgagtacaagtacatcaacgtcggccaggacggctccgttacctgggagtccgaccccaaccacacctacaccgttcctgctgtcgcttgcgtcacccaggttgtcaaggaggacacctggcagtct